SEQUENCE LISTING (421 sequences)

SEQ ID NO: 1 - protein, 25 aa, Artificial Sequence
  Description of Artificial Sequence Synthetic peptide
QVQLQQSGPQLVRPGASVKISCKAS

SEQ ID NO: 2 - protein, 23 aa, Artificial Sequence
  Description of Artificial Sequence Synthetic peptide
NIVMTQSPKSMYVSVGERVTLIC

SEQ ID NO: 3 - protein, 25 aa, Artificial Sequence
  Description of Artificial Sequence Synthetic peptide
QVQLQQSGPQLVRPGASVKISCKAS

SEQ ID NO: 4 - protein, 23 aa, Artificial Sequence
  Description of Artificial Sequence Synthetic peptide
NIVMTQSPKSMSVSVGERVTLSC

SEQ ID NO: 5 - protein, 25 aa, Artificial Sequence
  Description of Artificial Sequence Synthetic peptide
QVQLQQSGPQLVRPGASVKISCKAS

SEQ ID NO: 6 - protein, 23 aa, Artificial Sequence
  Description of Artificial Sequence Synthetic peptide
NIVMTQSPKSMSVSVGERVTLSC

SEQ ID NO: 7 - protein, 25 aa, Artificial Sequence
  Description of Artificial Sequence Synthetic peptide
EVQLQQSGAEFVKPGASVKLSCTAS

SEQ ID NO: 8 - protein, 23 aa, Artificial Sequence
  Description of Artificial Sequence Synthetic peptide
DIQMNQSPSSLSASLGDTITITC

SEQ ID NO: 9 - protein, 25 aa, Artificial Sequence
  Description of Artificial Sequence Synthetic peptide
QMQLQQSGPQLVRPGASVKISCKTS

SEQ ID NO: 10 - protein, 23 aa, Artificial Sequence
  Description of Artificial Sequence Synthetic peptide
NIVMTQSPRSMSMSVGERVTLSC

SEQ ID NO: 11 - protein, 25 aa, Artificial Sequence
  Description of Artificial Sequence Synthetic peptide
QVQLQQSGPQLVRPGASVKISCKAS

SEQ ID NO: 12 - protein, 23 aa, Artificial Sequence
  Description of Artificial Sequence Synthetic peptide
NIVMTQSPKSMSVSVGERVTMNC

SEQ ID NO: 13 - protein, 25 aa, Artificial Sequence
  Description of Artificial Sequence Synthetic peptide
QMQLQQSGPQLVRPGASVKISCKTS

SEQ ID NO: 14 - protein, 25 aa, Artificial Sequence
  Description of Artificial Sequence Synthetic peptide
QMQLQQSGPQLVRPGASVKISCKTS

SEQ ID NO: 15 - protein, 25 aa, Artificial Sequence
  Description of Artificial Sequence Synthetic peptide
QMQLQQSGPQLVRPGASVKISCKTS

SEQ ID NO: 16 - protein, 25 aa, Artificial Sequence
  Description of Artificial Sequence Synthetic peptide
QMQLQQSGPQLVRPGASVKISCKTS

SEQ ID NO: 17 - protein, 25 aa, Artificial Sequence
  Description of Artificial Sequence Synthetic peptide
QMQLQQSGPQLVRPGASVKISCKTS

SEQ ID NO: 18 - protein, 25 aa, Artificial Sequence [sequence omitted]

SEQ ID NO: 19 - protein, 25 aa, Artificial Sequence
  Description of Artificial Sequence Synthetic peptide
EVQLVQSGAEVKKPGSSVKVSCKAS

SEQ ID NO: 20 - protein, 25 aa, Artificial Sequence
  Description of Artificial Sequence Synthetic peptide
EVQLVQSGAEVKKPGSSVKVSCKAS

SEQ ID NO: 21 - protein, 25 aa, Artificial Sequence
  Description of Artificial Sequence Synthetic peptide
EVQLVQSGAEVKKPGSSVKVSCKTS

SEQ ID NO: 22 - protein, 25 aa, Artificial Sequence
  Description of Artificial Sequence Synthetic peptide
EVQLVQSGAEVKKPGSSVKVSCKAS

SEQ ID NO: 23 - protein, 25 aa, Artificial Sequence
  Description of Artificial Sequence Synthetic peptide
EVQLVQSGAEVKKPGSSVKVSCKAS

SEQ ID NO: 24 - protein, 25 aa, Artificial Sequence
  Description of Artificial Sequence Synthetic peptide
EVQLVQSGAEVKKPGSSVKVSCKAS

SEQ ID NO: 25 - protein, 25 aa, Artificial Sequence
  Description of Artificial Sequence Synthetic peptide
EVQLVQSGAEVKKPGSSVKVSCKAS

SEQ ID NO: 26 - protein, 23 aa, Artificial Sequence
  Description of Artificial Sequence Synthetic peptide
NIVMTQSPRSMSMSVGERVTLSC

SEQ ID NO: 27 - protein, 23 aa, Artificial Sequence
  Description of Artificial Sequence Synthetic peptide
EIVLTQSPATLSLSPGERATLSC

SEQ ID NO: 28 - protein, 23 aa, Artificial Sequence
  Description of Artificial Sequence Synthetic peptide
EIVLTQSPATLSLSPGERATLSC

SEQ ID NO: 29 - protein, 23 aa, Artificial Sequence
  Description of Artificial Sequence Synthetic peptide
EIVLTQSPATLSLSPGERATLSC

SEQ ID NO: 30 - protein, 23 aa, Artificial Sequence
  Description of Artificial Sequence Synthetic peptide
EIVLTQSPATLSLSPGERATLSC

SEQ ID NO: 31 - protein, 23 aa, Artificial Sequence
  Description of Artificial Sequence Synthetic peptide
EIVLTQSPATLSLSPGERVTLSC

SEQ ID NO: 32 - protein, 25 aa, Artificial Sequence
  Description of Artificial Sequence Synthetic peptide
EVQLQQSGAEFVKPGASVKLSCTAS

SEQ ID NO: 33 - protein, 25 aa, Artificial Sequence
  Description of Artificial Sequence Synthetic peptide
EVQLVQSGAEVKKPGSSVKVSCKAS

SEQ ID NO: 34 - protein, 25 aa, Artificial Sequence
  Description of Artificial Sequence Synthetic peptide
EVQLVQSGAEVKKPGSSVKVSCKAS

SEQ ID NO: 35 - protein, 25 aa, Artificial Sequence
  Description of Artificial Sequence Synthetic peptide
EVQLVQSGAEVKKPGSSVKVSCKAS

SEQ ID NO: 36 - protein, 23 aa, Artificial Sequence
  Description of Artificial Sequence Synthetic peptide
DIQMNQSPSSLSASLGDTITITC

SEQ ID NO: 37 - protein, 23 aa, Artificial Sequence
  Description of Artificial Sequence Synthetic peptide
DIQMTQSPSSLSASVGDRVTITC

SEQ ID NO: 38 - protein, 23 aa, Artificial Sequence
  Description of Artificial Sequence Synthetic peptide
DIQMTQSPSSLSASVGDRVTITC

SEQ ID NO: 39 - protein, 23 aa, Artificial Sequence
  Description of Artificial Sequence Synthetic peptide
DIQMTQSPSSLSASVGDRITITC

SEQ ID NO: 40 - protein, 25 aa, Artificial Sequence
  Description of Artificial Sequence Synthetic peptide
QVQLQQSGPQLVRPGASVKISCKAS

SEQ ID NO: 41 - protein, 25 aa, Artificial Sequence [sequence omitted]

SEQ ID NO: 42 - protein, 25 aa, Artificial Sequence
  Description of Artificial Sequence Synthetic peptide
EVQLVQSGAEVKKPGSSVKVSCKAS

SEQ ID NO: 43 - protein, 25 aa, Artificial Sequence
  Description of Artificial Sequence Synthetic peptide
EVQLVQSGAEVKKPGSSVKVSCKAS

SEQ ID NO: 44 - protein, 25 aa, Artificial Sequence
  Description of Artificial Sequence Synthetic peptide
EVQLVQSGAEVKKPGSSVKVSCKAS

SEQ ID NO: 45 - protein, 23 aa, Artificial Sequence
  Description of Artificial Sequence Synthetic peptide
NIVMTQSPKSMSVSVGERVTLSC

SEQ ID NO: 46 - protein, 23 aa, Artificial Sequence
  Description of Artificial Sequence Synthetic peptide
EIVMTQSPATLSVSPGERATLSC

SEQ ID NO: 47 - protein, 23 aa, Artificial Sequence
  Description of Artificial Sequence Synthetic peptide
EIVMTQSPATLSVSPGERATLSC

SEQ ID NO: 48 - protein, 23 aa, Artificial Sequence
  Description of Artificial Sequence Synthetic peptide
EIVMTQSPATLSVSPGERVTLSC

SEQ ID NO: 49 - protein, 10 aa, Artificial Sequence
  Description of Artificial Sequence Synthetic peptide
GYSFTNYWMH

SEQ ID NO: 50 - protein, 11 aa, Artificial Sequence
  Description of Artificial Sequence Synthetic peptide
RASEIVGTYVS

SEQ ID NO: 51 - protein, 10 aa, Artificial Sequence
  Description of Artificial Sequence Synthetic peptide
GYSFTNHWMH

SEQ ID NO: 52 - protein, 11 aa, Artificial Sequence
  Description of Artificial Sequence Synthetic peptide
RASDIVGTYVS

SEQ ID NO: 53 - protein, 10 aa, Artificial Sequence
  Description of Artificial Sequence Synthetic peptide
GYSFTNYWMH

SEQ ID NO: 54 - protein, 11 aa, Artificial Sequence
  Description of Artificial Sequence Synthetic peptide
RASEIVGTYVS

SEQ ID NO: 55 - protein, 10 aa, Artificial Sequence
  Description of Artificial Sequence Synthetic peptide
GFNIEDTYMH

SEQ ID NO: 56 - protein, 11 aa, Artificial Sequence
  Description of Artificial Sequence Synthetic peptide
HASQNINVWLS

SEQ ID NO: 57 - protein, 10 aa, Artificial Sequence
  Description of Artificial Sequence Synthetic peptide
GYSFTHHWIH

SEQ ID NO: 58 - protein, 11 aa, Artificial Sequence
  Description of Artificial Sequence Synthetic peptide
KASENVGTYIS

SEQ ID NO: 59 - protein, 10 aa, Artificial Sequence
  Description of Artificial Sequence Synthetic peptide
GYSFTNNWMH

SEQ ID NO: 60 - protein, 11 aa, Artificial Sequence
  Description of Artificial Sequence Synthetic peptide
RASEIVGTYVS

SEQ ID NO: 61 - protein, 10 aa, Artificial Sequence
  Description of Artificial Sequence Synthetic peptide
GYSFTHHWIH

SEQ ID NO: 62 - protein, 10 aa, Artificial Sequence
  Description of Artificial Sequence Synthetic peptide
GYSFTHHWIH

SEQ ID NO: 63 - protein, 10 aa, Artificial Sequence
  Description of Artificial Sequence Synthetic peptide
GYSFTHHWIH

SEQ ID NO: 64 - protein, 10 aa, Artificial Sequence
  Description of Artificial Sequence Synthetic peptide
GYSFTHHWIH

SEQ ID NO: 65 - protein, 10 aa, Artificial Sequence
  Description of Artificial Sequence Synthetic peptide
GYSFTHHWIH

SEQ ID NO: 66 - protein, 10 aa, Artificial Sequence
  Description of Artificial Sequence Synthetic peptide
GYSFTHHWIH

SEQ ID NO: 67 - protein, 10 aa, Artificial Sequence
  Description of Artificial Sequence Synthetic peptide
GYSFTHHWIH

SEQ ID NO: 68 - protein, 10 aa, Artificial Sequence
  Description of Artificial Sequence Synthetic peptide
GYSFTHHWIH

SEQ ID NO: 69 - protein, 10 aa, Artificial Sequence
  Description of Artificial Sequence Synthetic peptide
GYSFTHHWIH

SEQ ID NO: 70 - protein, 10 aa, Artificial Sequence
  Description of Artificial Sequence Synthetic peptide
GYSFTHHWIH

SEQ ID NO: 71 - protein, 10 aa, Artificial Sequence
  Description of Artificial Sequence Synthetic peptide
GYSFTHHWIH

SEQ ID NO: 72 - protein, 10 aa, Artificial Sequence
  Description of Artificial Sequence Synthetic peptide
GYSFTHHWIH

SEQ ID NO: 73 - protein, 10 aa, Artificial Sequence
  Description of Artificial Sequence Synthetic peptide
GYSFTHHWIH

SEQ ID NO: 74 - protein, 11 aa, Artificial Sequence
  Description of Artificial Sequence Synthetic peptide
KASENVGTYIS

SEQ ID NO: 75 - protein, 11 aa, Artificial Sequence
  Description of Artificial Sequence Synthetic peptide
KASENVGTYIS

SEQ ID NO: 76 - protein, 11 aa, Artificial Sequence
  Description of Artificial Sequence Synthetic peptide
RASENVGTYIS

SEQ ID NO: 77 - protein, 11 aa, Artificial Sequence
  Description of Artificial Sequence Synthetic peptide
RASENVGTYIS

SEQ ID NO: 78 - protein, 11 aa, Artificial Sequence
  Description of Artificial Sequence Synthetic peptide
RASENVGTYIS

SEQ ID NO: 79 - protein, 11 aa, Artificial Sequence
  Description of Artificial Sequence Synthetic peptide
RASENVGTYIS

SEQ ID NO: 80 - protein, 10 aa, Artificial Sequence
  Description of Artificial Sequence Synthetic peptide
GFNIEDTYMH

SEQ ID NO: 81 - protein, 10 aa, Artificial Sequence
  Description of Artificial Sequence Synthetic peptide
GFNIEDTYMH

SEQ ID NO: 82 - protein, 10 aa, Artificial Sequence
  Description of Artificial Sequence Synthetic peptide
GFNIEDTYMH

SEQ ID NO: 83 - protein, 10 aa, Artificial Sequence
  Description of Artificial Sequence Synthetic peptide
GFNIEDTYMH

SEQ ID NO: 84 - protein, 11 aa, Artificial Sequence
  Description of Artificial Sequence Synthetic peptide
HASQNINVWLS

SEQ ID NO: 85 - protein, 11 aa, Artificial Sequence
  Description of Artificial Sequence Synthetic peptide
HASQNINVWLS

SEQ ID NO: 86 - protein, 11 aa, Artificial Sequence
  Description of Artificial Sequence Synthetic peptide
RASQNINVWLS

SEQ ID NO: 87 - protein, 11 aa, Artificial Sequence
  Description of Artificial Sequence Synthetic peptide
RASQNINVWLS

SEQ ID NO: 88 - protein, 10 aa, Artificial Sequence
  Description of Artificial Sequence Synthetic peptide
GYSFTNHWMH

SEQ ID NO: 89 - protein, 10 aa, Artificial Sequence
  Description of Artificial Sequence Synthetic peptide
GYSFTNHWMH

SEQ ID NO: 90 - protein, 10 aa, Artificial Sequence
  Description of Artificial Sequence Synthetic peptide
GYSFTNHWMH

SEQ ID NO: 91 - protein, 10 aa, Artificial Sequence
  Description of Artificial Sequence Synthetic peptide
GYSFTNHWMH

SEQ ID NO: 92 - protein, 10 aa, Artificial Sequence
  Description of Artificial Sequence Synthetic peptide
GYSFTNHWMH

SEQ ID NO: 93 - protein, 11 aa, Artificial Sequence
  Description of Artificial Sequence Synthetic peptide
RASDIVGTYVS

SEQ ID NO: 94 - protein, 11 aa, Artificial Sequence
  Description of Artificial Sequence Synthetic peptide
RASDIVGTYVS

SEQ ID NO: 95 - protein, 11 aa, Artificial Sequence
  Description of Artificial Sequence Synthetic peptide
RASDIVGTYVS

SEQ ID NO: 96 - protein, 11 aa, Artificial Sequence
  Description of Artificial Sequence Synthetic peptide
RASDIVGTYVS

SEQ ID NO: 97 - protein, 14 aa, Artificial Sequence
  Description of Artificial Sequence Synthetic peptide
WMKQRPGQGLEWIG

SEQ ID NO: 98 - protein, 15 aa, Artificial Sequence
  Description of Artificial Sequence Synthetic peptide
WYQQKPEQSPKLLIY

SEQ ID NO: 99 - protein, 14 aa, Artificial Sequence
  Description of Artificial Sequence Synthetic peptide
WMKQRPGQGLEWIG

SEQ ID NO: 100 - protein, 15 aa, Artificial Sequence
  Description of Artificial Sequence Synthetic peptide
WYQQKPEQSPKLLIY

SEQ ID NO: 101 - protein, 14 aa, Artificial Sequence
  Description of Artificial Sequence Synthetic peptide
WMKQRPGQGLEWIG

SEQ ID NO: 102 - protein, 15 aa, Artificial Sequence
  Description of Artificial Sequence Synthetic peptide
WYQQKPEQSPKLLIY

SEQ ID NO: 103 - protein, 14 aa, Artificial Sequence
  Description of Artificial Sequence Synthetic peptide
WVKQRPEQGLEWIG

SEQ ID NO: 104 - protein, 15 aa, Artificial Sequence
  Description of Artificial Sequence Synthetic peptide
WYQQKPGNIPKLLIY

SEQ ID NO: 105 - protein, 14 aa, Artificial Sequence
  Description of Artificial Sequence Synthetic peptide
WMKQRPGQGLEWIG

SEQ ID NO: 106 - protein, 15 aa, Artificial Sequence
  Description of Artificial Sequence Synthetic peptide
WYQQKPDQSPKLLIY

SEQ ID NO: 107 - protein, 14 aa, Artificial Sequence
  Description of Artificial Sequence Synthetic peptide
WMKQRPGQGLEWIG

SEQ ID NO: 108 - protein, 15 aa, Artificial Sequence
  Description of Artificial Sequence Synthetic peptide
WYQQKPEQSPKLLIY

SEQ ID NO: 109 - protein, 14 aa, Artificial Sequence
  Description of Artificial Sequence Synthetic peptide
WMKQRPGQGLEWIG

SEQ ID NO: 110 - protein, 14 aa, Artificial Sequence
  Description of Artificial Sequence Synthetic peptide
WMKQRPGQGLEWIG

SEQ ID NO: 111 - protein, 14 aa, Artificial Sequence
  Description of Artificial Sequence Synthetic peptide
WMKQRPGQGLEWIG

SEQ ID NO: 112 - protein, 14 aa, Artificial Sequence
  Description of Artificial Sequence Synthetic peptide
WMKQRPGQGLEWIG

SEQ ID NO: 113 - protein, 14 aa, Artificial Sequence
  Description of Artificial Sequence Synthetic peptide
WMKQRPGQGLEWIG

SEQ ID NO: 114 - protein, 14 aa, Artificial Sequence
  Description of Artificial Sequence Synthetic peptide
WVRQAPGQGLEWMG

SEQ ID NO: 115 - protein, 14 aa, Artificial Sequence
  Description of Artificial Sequence Synthetic peptide
WMRQAPGQGLEWIG

SEQ ID NO: 116 - protein, 14 aa, Artificial Sequence
  Description of Artificial Sequence Synthetic peptide
WMRQAPGQGLEWIG

SEQ ID NO: 117 - protein, 14 aa, Artificial Sequence
  Description of Artificial Sequence Synthetic peptide
WMKQAPGQGLEWIG

SEQ ID NO: 118 - protein, 14 aa, Artificial Sequence
  Description of Artificial Sequence Synthetic peptide
WVRQAPGQGLEWMG

SEQ ID NO: 119 - protein, 14 aa, Artificial Sequence
  Description of Artificial Sequence Synthetic peptide
WVRQAPGQGLEWMG

SEQ ID NO: 120 - protein, 14 aa, Artificial Sequence
  Description of Artificial Sequence Synthetic peptide
WVRQAPGQGLEWMG

SEQ ID NO: 121 - protein, 14 aa, Artificial Sequence
  Description of Artificial Sequence Synthetic peptide
WVRQAPGQGLEWMG

SEQ ID NO: 122 - protein, 15 aa, Artificial Sequence
  Description of Artificial Sequence Synthetic peptide
WYQQKPDQSPKLLIY

SEQ ID NO: 123 - protein, 15 aa, Artificial Sequence
  Description of Artificial Sequence Synthetic peptide
WYQQKPGQAPRLLIY

SEQ ID NO: 124 - protein, 15 aa, Artificial Sequence
  Description of Artificial Sequence Synthetic peptide
WYQQKPGQAPRLLIY

SEQ ID NO: 125 - protein, 15 aa, Artificial Sequence
  Description of Artificial Sequence Synthetic peptide
WYQQKPGQAPRLLIY

SEQ ID NO: 126 - protein, 15 aa, Artificial Sequence
  Description of Artificial Sequence Synthetic peptide
WYQQKPGQAPRLLIY

SEQ ID NO: 127 - protein, 15 aa, Artificial Sequence
  Description of Artificial Sequence Synthetic peptide
WYQQKPGQAPRLLIY

SEQ ID NO: 128 - protein, 14 aa, Artificial Sequence
  Description of Artificial Sequence Synthetic peptide
WVKQRPEQGLEWIG

SEQ ID NO: 129 - protein, 14 aa, Artificial Sequence
  Description of Artificial Sequence Synthetic peptide
WVRQAPGQGLEWMG

SEQ ID NO: 130 - protein, 14 aa, Artificial Sequence
  Description of Artificial Sequence Synthetic peptide
WVRQAPGQGLEWIG

SEQ ID NO: 131 - protein, 14 aa, Artificial Sequence
  Description of Artificial Sequence Synthetic peptide
WVRQAPGQGLEWIG

SEQ ID NO: 132 - protein, 15 aa, Artificial Sequence
  Description of Artificial Sequence Synthetic peptide
WYQQKPGNIPKLLIY

SEQ ID NO: 133 - protein, 15 aa, Artificial Sequence
  Description of Artificial Sequence Synthetic peptide
WYQQKPGKAPKLLIY

SEQ ID NO: 134 - protein, 15 aa, Artificial Sequence
  Description of Artificial Sequence Synthetic peptide
WYQQKPGKAPKLLIY

SEQ ID NO: 135 - protein, 15 aa, Artificial Sequence
  Description of Artificial Sequence Synthetic peptide
WYQQKPGKAPKLLIY

SEQ ID NO: 136 - protein, 14 aa, Artificial Sequence
  Description of Artificial Sequence Synthetic peptide
WMKQRPGQGLEWIG

SEQ ID NO: 137 - protein, 14 aa, Artificial Sequence
  Description of Artificial Sequence Synthetic peptide
WVRQAPGQGLEWMG

SEQ ID NO: 138 - protein, 14 aa, Artificial Sequence
  Description of Artificial Sequence Synthetic peptide
WMRQAPGQGLEWIG

SEQ ID NO: 139 - protein, 14 aa, Artificial Sequence
  Description of Artificial Sequence Synthetic peptide
WMRQAPGQGLEWIG

SEQ ID NO: 140 - protein, 14 aa, Artificial Sequence
  Description of Artificial Sequence Synthetic peptide
WMKQAPGQGLEWIG

SEQ ID NO: 141 - protein, 15 aa, Artificial Sequence
  Description of Artificial Sequence Synthetic peptide
WYQQKPEQSPKLLIY

SEQ ID NO: 142 - protein, 15 aa, Artificial Sequence
  Description of Artificial Sequence Synthetic peptide
WYQQKPGQAPRLLIY

SEQ ID NO: 143 - protein, 15 aa, Artificial Sequence
  Description of Artificial Sequence Synthetic peptide
WYQQKPGQAPRLLIY

SEQ ID NO: 144 - protein, 15 aa, Artificial Sequence
  Description of Artificial Sequence Synthetic peptide
WYQQKPGQAPRLLIY

SEQ ID NO: 145 - protein, 17 aa, Artificial Sequence
  Description of Artificial Sequence Synthetic peptide
MIDPSDSETRLNQQFKD

SEQ ID NO: 146 - protein, 7 aa, Artificial Sequence
  Description of Artificial Sequence Synthetic peptide
GASNRYT

SEQ ID NO: 147 - protein, 17 aa, Artificial Sequence
  Description of Artificial Sequence Synthetic peptide
MIDPSDSETRLNQQFKD

SEQ ID NO: 148 - protein, 7 aa, Artificial Sequence
  Description of Artificial Sequence Synthetic peptide
GASNRYT

SEQ ID NO: 149 - protein, 17 aa, Artificial Sequence
  Description of Artificial Sequence Synthetic peptide
MIDPSDSETRLNQQFKD

SEQ ID NO: 150 - protein, 7 aa, Artificial Sequence
  Description of Artificial Sequence Synthetic peptide
GASNRFT

SEQ ID NO: 151 - protein, 17 aa, Artificial Sequence
  Description of Artificial Sequence Synthetic peptide
MIDPANGKTKYGPRFQD

SEQ ID NO: 152 - protein, 7 aa, Artificial Sequence
  Description of Artificial Sequence Synthetic peptide
KASNLHT

SEQ ID NO: 153 - protein, 17 aa, Artificial Sequence
  Description of Artificial Sequence Synthetic peptide
MIDPSDSETRLSQKFKD

SEQ ID NO: 154 - protein, 7 aa, Artificial Sequence
  Description of Artificial Sequence Synthetic peptide
GASNRYT

SEQ ID NO: 155 - protein, 17 aa, Artificial Sequence
  Description of Artificial Sequence Synthetic peptide
MIDPSDSETRLNQQFRD

SEQ ID NO: 156 - protein, 7 aa, Artificial Sequence
  Description of Artificial Sequence Synthetic peptide
GAFNRYT

SEQ ID NO: 157 - protein, 17 aa, Artificial Sequence
  Description of Artificial Sequence Synthetic peptide
MIDPSDSETRLSQKFKD

SEQ ID NO: 158 - protein, 17 aa, Artificial Sequence
  Description of Artificial Sequence Synthetic peptide
IIDPSDSETRLSQKFKD

SEQ ID NO: 159 - protein, 17 aa, Artificial Sequence
  Description of Artificial Sequence Synthetic peptide
MIEPSDSETRLSQKFKD

SEQ ID NO: 160 - protein, 17 aa, Artificial Sequence
  Description of Artificial Sequence Synthetic peptide
MISPSDSETRLSQKFKD

SEQ ID NO: 161 - protein, 17 aa, Artificial Sequence
  Description of Artificial Sequence Synthetic peptide
MIDASDSETRLSQKFKD

SEQ ID NO: 162 - protein, 17 aa, Artificial Sequence
  Description of Artificial Sequence Synthetic peptide
MIDPSDSETRLSQKFKD

SEQ ID NO: 163 - protein, 17 aa, Artificial Sequence
  Description of Artificial Sequence Synthetic peptide
MIDPSDSETRLSQKFKD

SEQ ID NO: 164 - protein, 17 aa, Artificial Sequence
  Description of Artificial Sequence Synthetic peptide
MIDPSDSETRLSQKFKD

SEQ ID NO: 165 - protein, 17 aa, Artificial Sequence
  Description of Artificial Sequence Synthetic peptide
MIDPSDSETRLSQKFKD

SEQ ID NO: 166 - protein, 17 aa, Artificial Sequence
  Description of Artificial Sequence Synthetic peptide
MIDPSDSETRLSQKFKD

SEQ ID NO: 167 - protein, 17 aa, Artificial Sequence
  Description of Artificial Sequence Synthetic peptide
MIEPSDSETRLSQKFKD

SEQ ID NO: 168 - protein, 17 aa, Artificial Sequence
  Description of Artificial Sequence Synthetic peptide
MISPSDSETRLSQKFKD

SEQ ID NO: 169 - protein, 17 aa, Artificial Sequence
  Description of Artificial Sequence Synthetic peptide
MIDASDSETRLSQKFKD

SEQ ID NO: 170 - protein, 7 aa, Artificial Sequence
  Description of Artificial Sequence Synthetic peptide
GASNRYT

SEQ ID NO: 171 - protein, 7 aa, Artificial Sequence
  Description of Artificial Sequence Synthetic peptide
GASNRYT

SEQ ID NO: 172 - protein, 7 aa, Artificial Sequence
  Description of Artificial Sequence Synthetic peptide
GASNRYT

SEQ ID NO: 173 - protein, 7 aa, Artificial Sequence
  Description of Artificial Sequence Synthetic peptide
GASNRYT

SEQ ID NO: 174 - protein, 7 aa, Artificial Sequence
  Description of Artificial Sequence Synthetic peptide
GASNRYT

SEQ ID NO: 175 - protein, 7 aa, Artificial Sequence
  Description of Artificial Sequence Synthetic peptide
GASNRYT

SEQ ID NO: 176 - protein, 17 aa, Artificial Sequence
  Description of Artificial Sequence Synthetic peptide
MIDPANGKTKYGPRFQD

SEQ ID NO: 177 - protein, 17 aa, Artificial Sequence
  Description of Artificial Sequence Synthetic peptide
MIDPANGKTKYGPRFQD

SEQ ID NO: 178 - protein, 17 aa, Artificial Sequence
  Description of Artificial Sequence Synthetic peptide
MIDPANGKTKYGPRFQD

SEQ ID NO: 179 - protein, 17 aa, Artificial Sequence
  Description of Artificial Sequence Synthetic peptide
MIDPANGKTKYGPRFQD

SEQ ID NO: 180 - protein, 7 aa, Artificial Sequence
  Description of Artificial Sequence Synthetic peptide
KASNLHT

SEQ ID NO: 181 - protein, 7 aa, Artificial Sequence
  Description of Artificial Sequence Synthetic peptide
KASNLHT

SEQ ID NO: 182 - protein, 7 aa, Artificial Sequence
  Description of Artificial Sequence Synthetic peptide
KASNLHT

SEQ ID NO: 183 - protein, 7 aa, Artificial Sequence
  Description of Artificial Sequence Synthetic peptide
KASNLHT

SEQ ID NO: 184 - protein, 17 aa, Artificial Sequence
  Description of Artificial Sequence Synthetic peptide
MIDPSDSETRLNQQFKD

SEQ ID NO: 185 - protein, 17 aa, Artificial Sequence
  Description of Artificial Sequence Synthetic peptide
MIDPSDSETRLNQQFKD

SEQ ID NO: 186 - protein, 17 aa, Artificial Sequence
  Description of Artificial Sequence Synthetic peptide
MIDPSDSETRLNQQFKD

SEQ ID NO: 187 - protein, 17 aa, Artificial Sequence
  Description of Artificial Sequence Synthetic peptide
MIDPSDSETRLNQQFKD

SEQ ID NO: 188 - protein, 17 aa, Artificial Sequence
  Description of Artificial Sequence Synthetic peptide
MIDPSDSETRLNQQFKD

SEQ ID NO: 189 - protein, 7 aa, Artificial Sequence
  Description of Artificial Sequence Synthetic peptide
GASNRYT

SEQ ID NO: 190 - protein, 7 aa, Artificial Sequence
  Description of Artificial Sequence Synthetic peptide
GASNRYT

SEQ ID NO: 191 - protein, 7 aa, Artificial Sequence
  Description of Artificial Sequence Synthetic peptide
GASNRYT

SEQ ID NO: 192 - protein, 7 aa, Artificial Sequence
  Description of Artificial Sequence Synthetic peptide
GASNRYT

SEQ ID NO: 193 - protein, 32 aa, Artificial Sequence
  Description of Artificial Sequence Synthetic polypeptide
KATLAVDKSSSTAYMQLSSPTSEDSAVYYCAR

SEQ ID NO: 194 - protein, 32 aa, Artificial Sequence
  Description of Artificial Sequence Synthetic polypeptide
GVPDRFTGSRSATDFSLTISNVQAEDLADYLC

SEQ ID NO: 195 - protein, 32 aa, Artificial Sequence
  Description of Artificial Sequence Synthetic polypeptide
KATLTVDKSSSTAYMQLSSPTSEDSAVFYCAR

SEQ ID NO: 196 - protein, 32 aa, Artificial Sequence; identical to SEQ ID NO: 194
  Description of Artificial Sequence Synthetic polypeptide

SEQ ID NO: 197 - protein, 32 aa, Artificial Sequence
  Description of Artificial Sequence Synthetic polypeptide
KATLTVDKSSSTAYMQLSSPTSEDSAVYYCAR

SEQ ID NO: 198 - protein, 32 aa, Artificial Sequence; identical to SEQ ID NO: 194
  Description of Artificial Sequence Synthetic polypeptide

SEQ ID NO: 199 - protein, 32 aa, Artificial Sequence
  Description of Artificial Sequence Synthetic polypeptide
KATVTADTSSNTANLQLSSLTSEDTAVYYCAD

SEQ ID NO: 200 - protein, 32 aa, Artificial Sequence
  Description of Artificial Sequence Synthetic polypeptide
GVPSRFSGSGSGTGFTLTISSLQPEDIATYYC

SEQ ID NO: 201 - protein, 32 aa, Artificial Sequence
  Description of Artificial Sequence Synthetic polypeptide
KATLTVDASSSTAYMQLNSPTSEDSALYFCAR

SEQ ID NO: 202 - protein, 32 aa, Artificial Sequence
  Description of Artificial Sequence Synthetic polypeptide
GVPDRFTGSGSGTDFTLTISTVQAEDLADYHC

SEQ ID NO: 203 - protein, 32 aa, Artificial Sequence
  Description of Artificial Sequence Synthetic polypeptide
KATLTVDKTSSTAYMQLSSPTSEDSAVYYCAR

SEQ ID NO: 204 - protein, 32 aa, Artificial Sequence
  Description of Artificial Sequence Synthetic polypeptide
GVPDRFTGSRSGTDFSLNISNVQAEDLADYLC

SEQ ID NO: 205 - protein, 32 aa, Artificial Sequence; identical to SEQ ID NO: 201
  Description of Artificial Sequence Synthetic polypeptide

SEQ ID NO: 206 - protein, 32 aa, Artificial Sequence; identical to SEQ ID NO: 201
  Description of Artificial Sequence Synthetic polypeptide

SEQ ID NO: 207 - protein, 32 aa, Artificial Sequence; identical to SEQ ID NO: 201
  Description of Artificial Sequence Synthetic polypeptide

SEQ ID NO: 208 - protein, 32 aa, Artificial Sequence; identical to SEQ ID NO: 201
  Description of Artificial Sequence Synthetic polypeptide

SEQ ID NO: 209 - protein, 32 aa, Artificial Sequence; identical to SEQ ID NO: 201
  Description of Artificial Sequence Synthetic polypeptide

SEQ ID NO: 210 - protein, 32 aa, Artificial Sequence
  Description of Artificial Sequence Synthetic polypeptide
RVTITADKSTSTAYMELSSLRSEDTAVYYCAR

SEQ ID NO: 211 - protein, 32 aa, Artificial Sequence
  Description of Artificial Sequence Synthetic polypeptide
RVTITADKSTSTAYMELSSLRSEDTAVYFCAR

SEQ ID NO: 212 - protein, 32 aa, Artificial Sequence
  Description of Artificial Sequence Synthetic polypeptide
RATLTVDKSTSTAYMELSSLRSEDTAVYFCAR

SEQ ID NO: 213 - protein, 32 aa, Artificial Sequence
  Description of Artificial Sequence Synthetic polypeptide
KATLTVDKSTSTAYMELSSLRSEDTAVYFCAR

SEQ ID NO: 214 - protein, 32 aa, Artificial Sequence; identical to SEQ ID NO: 210
  Description of Artificial Sequence Synthetic polypeptide

SEQ ID NO: 215 - protein, 32 aa, Artificial Sequence; identical to SEQ ID NO: 210
  Description of Artificial Sequence Synthetic polypeptide

SEQ ID NO: 216 - protein, 32 aa, Artificial Sequence; identical to SEQ ID NO: 210
  Description of Artificial Sequence Synthetic polypeptide

SEQ ID NO: 217 - protein, 32 aa, Artificial Sequence; identical to SEQ ID NO: 210
  Description of Artificial Sequence Synthetic polypeptide

SEQ ID NO: 218 - protein, 32 aa, Artificial Sequence; identical to SEQ ID NO: 202
  Description of Artificial Sequence Synthetic polypeptide

SEQ ID NO: 219 - protein, 32 aa, Artificial Sequence
  Description of Artificial Sequence Synthetic polypeptide
GIPARFSGSGSGTDFTLTISSLEPEDFAVYYC

SEQ ID NO: 220 - protein, 32 aa, Artificial Sequence; identical to SEQ ID NO: 219
  Description of Artificial Sequence Synthetic polypeptide

SEQ ID NO: 221 - protein, 32 aa, Artificial Sequence; identical to SEQ ID NO: 219
  Description of Artificial Sequence Synthetic polypeptide

SEQ ID NO: 222 - protein, 32 aa, Artificial Sequence
  Description of Artificial Sequence Synthetic polypeptide
GVPARFSGSGSGTDFTLTISSLEPEDFAVYHC

SEQ ID NO: 223 - protein, 32 aa, Artificial Sequence
  Description of Artificial Sequence Synthetic polypeptide
GVPARFSGSGSGTDFTLTISSVEPEDFAVYHC

SEQ ID NO: 224 - protein, 32 aa, Artificial Sequence; identical to SEQ ID NO: 199
  Description of Artificial Sequence Synthetic polypeptide

SEQ ID NO: 225 - protein, 32 aa, Artificial Sequence; identical to SEQ ID NO: 210
  Description of Artificial Sequence Synthetic polypeptide

SEQ ID NO: 226 - protein, 32 aa, Artificial Sequence
  Description of Artificial Sequence Synthetic polypeptide
RVTITADKSTSTAYMELSSLRSEDTAVYYCAD

SEQ ID NO: 227 - protein, 32 aa, Artificial Sequence
  Description of Artificial Sequence Synthetic polypeptide
RATVTADKSTSTAYMELSSLRSEDTAVYYCAD

SEQ ID NO: 228 - protein, 32 aa, Artificial Sequence; identical to SEQ ID NO: 200
  Description of Artificial Sequence Synthetic polypeptide

SEQ ID NO: 229 - protein, 32 aa, Artificial Sequence
  Description of Artificial Sequence Synthetic polypeptide
GVPSRFSGSGSGTDFTLTISSLQPEDFATYYC

SEQ ID NO: 230 - protein, 32 aa, Artificial Sequence; identical to SEQ ID NO: 229
  Description of Artificial Sequence Synthetic polypeptide

SEQ ID NO: 231 - protein, 32 aa, Artificial Sequence; identical to SEQ ID NO: 229
  Description of Artificial Sequence Synthetic polypeptide

SEQ ID NO: 232 - protein, 32 aa, Artificial Sequence; identical to SEQ ID NO: 195
  Description of Artificial Sequence Synthetic polypeptide

SEQ ID NO: 233 - protein, 32 aa, Artificial Sequence; identical to SEQ ID NO: 210
  Description of Artificial Sequence Synthetic polypeptide

SEQ ID NO: 234 - protein, 32 aa, Artificial Sequence
  Description of Artificial Sequence Synthetic polypeptide
RVTITADKSTSTAYMELSSLRSEDTAVFYCAR

SEQ ID NO: 235 - protein, 32 aa, Artificial Sequence
  Description of Artificial Sequence Synthetic polypeptide
RATLTVDKSTSTAYMELSSLRSEDTAVFYCAR

SEQ ID NO: 236 - protein, 32 aa, Artificial Sequence
  Description of Artificial Sequence Synthetic polypeptide
KATLTVDKSTSTAYMELSSLRSEDTAVYYCAR

SEQ ID NO: 237 - protein, 32 aa, Artificial Sequence; identical to SEQ ID NO: 194
  Description of Artificial Sequence Synthetic polypeptide

SEQ ID NO: 238 - protein, 32 aa, Artificial Sequence
  Description of Artificial Sequence Synthetic polypeptide
GIPARFSGSGSGTEFTLTISSLQSEDFAVYYC

SEQ ID NO: 239 - protein, 32 aa, Artificial Sequence
  Description of Artificial Sequence Synthetic polypeptide
GVPARFSGSGSGTEFTLTISSLQSEDFAVYLC

SEQ ID NO: 240 - protein, 32 aa, Artificial Sequence
  Description of Artificial Sequence Synthetic polypeptide
GVPARFSGSGSGTEFTLTISSVQSEDFAVYLC

SEQ ID NO: 241 - protein, 8 aa, Artificial Sequence
  Description of Artificial Sequence Synthetic peptide
LGRYYFDY

SEQ ID NO: 242 - protein, 9 aa, Artificial Sequence
  Description of Artificial Sequence Synthetic peptide
GQSYDSPYT

SEQ ID NO: 243 - protein, 8 aa, Artificial Sequence
  Description of Artificial Sequence Synthetic peptide
LGRYYFDY

SEQ ID NO: 244 - protein, 9 aa, Artificial Sequence
  Description of Artificial Sequence Synthetic peptide
GQSYDSPYT

SEQ ID NO: 245 - protein, 8 aa, Artificial Sequence
  Description of Artificial Sequence Synthetic peptide
LGRYYFDF

SEQ ID NO: 246 - protein, 9 aa, Artificial Sequence
  Description of Artificial Sequence Synthetic peptide
GQSYDSPYT

SEQ ID NO: 247 - protein, 11 aa, Artificial Sequence
  Description of Artificial Sequence Synthetic peptide
GIGYYVGAMDY

SEQ ID NO: 248 - protein, 9 aa, Artificial Sequence
  Description of Artificial Sequence Synthetic peptide
QQGHSYPYT

SEQ ID NO: 249 - protein, 8 aa, Artificial Sequence
  Description of Artificial Sequence Synthetic peptide
LGRYYFDY

SEQ ID NO: 250 - protein, 9 aa, Artificial Sequence
  Description of Artificial Sequence Synthetic peptide
GESYGHLYT

SEQ ID NO: 251 - protein, 8 aa, Artificial Sequence
  Description of Artificial Sequence Synthetic peptide
LGRYYFDY

SEQ ID NO: 252 - protein, 9 aa, Artificial Sequence
  Description of Artificial Sequence Synthetic peptide
GQSYDSPYT

SEQ ID NO: 253 - protein, 8 aa, Artificial Sequence
  Description of Artificial Sequence Synthetic peptide
LGRYYFDY

SEQ ID NO: 254 - protein, 8 aa, Artificial Sequence
  Description of Artificial Sequence Synthetic peptide
LGRYYFDY

SEQ ID NO: 255 - protein, 8 aa, Artificial Sequence
  Description of Artificial Sequence Synthetic peptide
LGRYYFDY

SEQ ID NO: 256 - protein, 8 aa, Artificial Sequence
  Description of Artificial Sequence Synthetic peptide
LGRYYFDY

SEQ ID NO: 257 - protein, 8 aa, Artificial Sequence
  Description of Artificial Sequence Synthetic peptide
LGRYYFDY

SEQ ID NO: 258 - protein, 8 aa, Artificial Sequence
  Description of Artificial Sequence Synthetic peptide
LGRYYFDY

SEQ ID NO: 259 - protein, 8 aa, Artificial Sequence
  Description of Artificial Sequence Synthetic peptide
LGRYYFDY

SEQ ID NO: 260 - protein, 8 aa, Artificial Sequence
  Description of Artificial Sequence Synthetic peptide
LGRYYFDY

SEQ ID NO: 261 - protein, 8 aa, Artificial Sequence
  Description of Artificial Sequence Synthetic peptide
LGRYYFDY

SEQ ID NO: 262 - protein, 8 aa, Artificial Sequence
  Description of Artificial Sequence Synthetic peptide
LGRYYFDY

SEQ ID NO: 263 - protein, 8 aa, Artificial Sequence
  Description of Artificial Sequence Synthetic peptide
LGRYYFDY

SEQ ID NO: 264 - protein, 8 aa, Artificial Sequence
  Description of Artificial Sequence Synthetic peptide
LGRYYFDY

SEQ ID NO: 265 - protein, 8 aa, Artificial Sequence
  Description of Artificial Sequence Synthetic peptide
LGRYYFDY

SEQ ID NO: 266 - protein, 9 aa, Artificial Sequence
  Description of Artificial Sequence Synthetic peptide
GESYGHLYT

SEQ ID NO: 267 - protein, 9 aa, Artificial Sequence
  Description of Artificial Sequence Synthetic peptide
GESYGHLYT

SEQ ID NO: 268 - protein, 9 aa, Artificial Sequence
  Description of Artificial Sequence Synthetic peptide
GESYGHLYT

SEQ ID NO: 269 - protein, 9 aa, Artificial Sequence
  Description of Artificial Sequence Synthetic peptide
GESYGHLYT

SEQ ID NO: 270 - protein, 9 aa, Artificial Sequence
  Description of Artificial Sequence Synthetic peptide
GESYGHLYT

SEQ ID NO: 271 - protein, 9 aa, Artificial Sequence
  Description of Artificial Sequence Synthetic peptide
GESYGHLYT

SEQ ID NO: 272 - protein, 11 aa, Artificial Sequence
  Description of Artificial Sequence Synthetic peptide
GIGYYVGAMDY

SEQ ID NO: 273 - protein, 11 aa, Artificial Sequence
  Description of Artificial Sequence Synthetic peptide
GIGYYVGAMDY

SEQ ID NO: 274 - protein, 11 aa, Artificial Sequence
  Description of Artificial Sequence Synthetic peptide
GIGYYVGAMDY

SEQ ID NO: 275 - protein, 11 aa, Artificial Sequence
  Description of Artificial Sequence Synthetic peptide
GIGYYVGAMDY

SEQ ID NO: 276 - protein, 9 aa, Artificial Sequence
  Description of Artificial Sequence Synthetic peptide
QQGHSYPYT

SEQ ID NO: 277 - protein, 9 aa, Artificial Sequence
  Description of Artificial Sequence Synthetic peptide
QQGHSYPYT

SEQ ID NO: 278 - protein, 9 aa, Artificial Sequence
  Description of Artificial Sequence Synthetic peptide
QQGHSYPYT

SEQ ID NO: 279 - protein, 9 aa, Artificial Sequence
  Description of Artificial Sequence Synthetic peptide
QQGHSYPYT

SEQ ID NO: 280 - protein, 8 aa, Artificial Sequence
  Description of Artificial Sequence Synthetic peptide
LGRYYFDY

SEQ ID NO: 281 - protein, 8 aa, Artificial Sequence
  Description of Artificial Sequence Synthetic peptide
LGRYYFDY

SEQ ID NO: 282 - protein, 8 aa, Artificial Sequence
  Description of Artificial Sequence Synthetic peptide
LGRYYFDY

SEQ ID NO: 283 - protein, 8 aa, Artificial Sequence
  Description of Artificial Sequence Synthetic peptide
LGRYYFDY

SEQ ID NO: 284 - protein, 8 aa, Artificial Sequence
  Description of Artificial Sequence Synthetic peptide
LGRYYFDY

SEQ ID NO: 285 - protein, 9 aa, Artificial Sequence
  Description of Artificial Sequence Synthetic peptide
GQSYDSPYT

SEQ ID NO: 286 - protein, 9 aa, Artificial Sequence
  Description of Artificial Sequence Synthetic peptide
GQSYDSPYT

SEQ ID NO: 287 - protein, 9 aa, Artificial Sequence
  Description of Artificial Sequence Synthetic peptide
GQSYDSPYT

SEQ ID NO: 288 - protein, 9 aa, Artificial Sequence
  Description of Artificial Sequence Synthetic peptide
GQSYDSPYT

SEQ ID NO: 289 - protein, 11 aa, Artificial Sequence
  Description of Artificial Sequence Synthetic peptide
WGQGTTLTVSS

SEQ ID NO: 290 - protein, 10 aa, Artificial Sequence
  Description of Artificial Sequence Synthetic peptide
FGGGTKLEIK

SEQ ID NO: 291 - protein, 11 aa, Artificial Sequence
  Description of Artificial Sequence Synthetic peptide
WGQGTTLTVSS

SEQ ID NO: 292 - protein, 10 aa, Artificial Sequence
  Description of Artificial Sequence Synthetic peptide
FGGGTKLEIK

SEQ ID NO: 293 - protein, 11 aa, Artificial Sequence
  Description of Artificial Sequence Synthetic peptide
WGQGTTLTVSS

SEQ ID NO: 294 - protein, 10 aa, Artificial Sequence
  Description of Artificial Sequence Synthetic peptide
FGGGTKLEIK

SEQ ID NO: 295 - protein, 11 aa, Artificial Sequence
  Description of Artificial Sequence Synthetic peptide
WGQGTSVTVSS

SEQ ID NO: 296 - protein, 10 aa, Artificial Sequence
  Description of Artificial Sequence Synthetic peptide
FGGGTKLEIK

SEQ ID NO: 297 - protein, 11 aa, Artificial Sequence
  Description of Artificial Sequence Synthetic peptide
WGQGTTLTVSS

SEQ ID NO: 298 - protein, 10 aa, Artificial Sequence
  Description of Artificial Sequence Synthetic peptide
FGGGTKLEIK

SEQ ID NO: 299 - protein, 11 aa, Artificial Sequence
  Description of Artificial Sequence Synthetic peptide
WGLGTTLTVSS

SEQ ID NO: 300 - protein, 10 aa, Artificial Sequence
  Description of Artificial Sequence Synthetic peptide
FGGGTKLEIK

SEQ ID NO: 301 - protein, 11 aa, Artificial Sequence
  Description of Artificial Sequence Synthetic peptide
WGQGTTLTVSS

SEQ ID NO: 302 - protein, 11 aa, Artificial Sequence
  Description of Artificial Sequence Synthetic peptide
WGQGTTLTVSS

SEQ ID NO: 303 - protein, 11 aa, Artificial Sequence
  Description of Artificial Sequence Synthetic peptide
WGQGTTLTVSS

SEQ ID NO: 304 - protein, 11 aa, Artificial Sequence
  Description of Artificial Sequence Synthetic peptide
WGQGTTLTVSS

SEQ ID NO: 305 - protein, 11 aa, Artificial Sequence
  Description of Artificial Sequence Synthetic peptide
WGQGTTLTVSS

SEQ ID NO: 306 - protein, 11 aa, Artificial Sequence
  Description of Artificial Sequence Synthetic peptide
WGQGTTVTVSS

SEQ ID NO: 307 - protein, 11 aa, Artificial Sequence
  Description of Artificial Sequence Synthetic peptide
WGQGTTVTVSS

SEQ ID NO: 308 - protein, 11 aa, Artificial Sequence
  Description of Artificial Sequence Synthetic peptide
WGQGTTVTVSS

SEQ ID NO: 309 - protein, 11 aa, Artificial Sequence
  Description of Artificial Sequence Synthetic peptide
WGQGTTVTVSS

SEQ ID NO: 310 - protein, 11 aa, Artificial Sequence
  Description of Artificial Sequence Synthetic peptide
WGQGTTVTVSS

SEQ ID NO: 311 - protein, 11 aa, Artificial Sequence
  Description of Artificial Sequence Synthetic peptide
WGQGTTVTVSS

SEQ ID NO: 312 - protein, 11 aa, Artificial Sequence
  Description of Artificial Sequence Synthetic peptide
WGQGTTVTVSS

SEQ ID NO: 313 - protein, 11 aa, Artificial Sequence
  Description of Artificial Sequence Synthetic peptide
WGQGTTVTVSS

SEQ ID NO: 314 - protein, 10 aa, Artificial Sequence
  Description of Artificial Sequence Synthetic peptide
FGGGTKLEIK

SEQ ID NO: 315 - protein, 10 aa, Artificial Sequence
  Description of Artificial Sequence Synthetic peptide
FGGGTKVEIK

SEQ ID NO: 316 - protein, 10 aa, Artificial Sequence
  Description of Artificial Sequence Synthetic peptide
FGGGTKVEIK

SEQ ID NO: 317 - protein, 10 aa, Artificial Sequence
  Description of Artificial Sequence Synthetic peptide
FGGGTKVEIK

SEQ ID NO: 318 - protein, 10 aa, Artificial Sequence
  Description of Artificial Sequence Synthetic peptide
FGGGTKVEIK

SEQ ID NO: 319 - protein, 10 aa, Artificial Sequence
  Description of Artificial Sequence Synthetic peptide
FGGGTKLEIK

SEQ ID NO: 320 - protein, 11 aa, Artificial Sequence
  Description of Artificial Sequence Synthetic peptide
WGQGTSVTVSS

SEQ ID NO: 321 - protein, 11 aa, Artificial Sequence
  Description of Artificial Sequence Synthetic peptide
WGQGTTVTVSS

SEQ ID NO: 322 - protein, 11 aa, Artificial Sequence
  Description of Artificial Sequence Synthetic peptide
WGQGTTVTVSS

SEQ ID NO: 323 - protein, 11 aa, Artificial Sequence
  Description of Artificial Sequence Synthetic peptide
WGQGTTVTVSS

SEQ ID NO: 324 - protein, 10 aa, Artificial Sequence
  Description of Artificial Sequence Synthetic peptide
FGGGTKLEIK

SEQ ID NO: 325 - protein, 10 aa, Artificial Sequence
  Description of Artificial Sequence Synthetic peptide
FGGGTKVEIK

SEQ ID NO: 326 - protein, 10 aa, Artificial Sequence
  Description of Artificial Sequence Synthetic peptide
FGGGTKVEIK

SEQ ID NO: 327 - protein, 10 aa, Artificial Sequence
  Description of Artificial Sequence Synthetic peptide
FGGGTKLEIK

SEQ ID NO: 328 - protein, 11 aa, Artificial Sequence
  Description of Artificial Sequence Synthetic peptide
WGQGTTLTVSS

SEQ ID NO: 329 - protein, 11 aa, Artificial Sequence
  Description of Artificial Sequence Synthetic peptide
WGQGTTVTVSS

SEQ ID NO: 330 - protein, 11 aa, Artificial Sequence
  Description of Artificial Sequence Synthetic peptide
WGQGTTVTVSS

SEQ ID NO: 331 - protein, 11 aa, Artificial Sequence
  Description of Artificial Sequence Synthetic peptide
WGQGTTVTVSS

SEQ ID NO: 332 - protein, 11 aa, Artificial Sequence
  Description of Artificial Sequence Synthetic peptide
WGQGTTVTVSS

SEQ ID NO: 333 - protein, 10 aa, Artificial Sequence
  Description of Artificial Sequence Synthetic peptide
FGGGTKLEIK

SEQ ID NO: 334 - protein, 10 aa, Artificial Sequence
  Description of Artificial Sequence Synthetic peptide
FGGGTKVEIK

SEQ ID NO: 335 - protein, 10 aa, Artificial Sequence
  Description of Artificial Sequence Synthetic peptide
FGGGTKVEIK

SEQ ID NO: 336 - protein, 10 aa, Artificial Sequence
  Description of Artificial Sequence Synthetic peptide
FGGGTKLEIK

SEQ ID NO: 337 - DNA, 351 bp, Artificial Sequence
  Description of Artificial Sequence Synthetic polynucleotide
caggtgcaactgcagcagtctgggcctcagctggttaggcctggggcttcagtgaagatatcctgcaaggcttctggttactcattcaccaactactggatgcactggatgaagcagaggcctggacaaggtcttgaatggattggcatgattgatccttccgatagtgagactaggttaaatcagcagttcaaggacaaggccacattggctgttgacaaatcctccagcacagcctacatgcaactcagcagcccgacatctgaggactctgcggtctattactgtgcaagattagggcggtattattttgactactggggccaaggcaccactctcacagtctcctca

SEQ ID NO: 338 - DNA, 321 bp, Artificial Sequence
  Description of Artificial Sequence Synthetic polynucleotide
aacattgtaatgacccaatctcccaaatccatgtacgtgtcagtcggggagagggtcaccttgatctgcagggccagtgagattgtgggcacttatgtttcctggtatcaacagaaaccagagcagtctcctaaattgctgatatacggggcatccaaccggtacactggggtccccgatcgcttcacaggcagtagatctgcaacagatttcagtctgaccatcagtaatgtgcaggctgaagaccttgcagattatctctgtggacagagttacgactctccgtacacgttcggaggggggaccaagctggaaataaaa

SEQ ID NO: 339 - DNA, 351 bp, Artificial Sequence
  Description of Artificial Sequence Synthetic polynucleotide
caggtgcaactgcagcagtctgggcctcagctagttaggcctggggcttcagtgaagatatcctgcaaggcttctggttactcattcaccaaccactggatgcactggatgaagcagaggcctggacaaggtcttgaatggattggcatgattgatccttccgatagtgagactaggttaaatcagcagttcaaggacaaggccacattgactgttgacaaatcctccagcacagcctacatgcaactcagcagcccgacatctgaggactctgcggtcttttactgtgcaagattagggcggtattattttgactactggggccaaggcaccactctcacagtctcctca

SEQ ID NO: 340 - DNA, 321 bp, Artificial Sequence
  Description of Artificial Sequence Synthetic polynucleotide
aacattgtaatgacccaatctcccaaatccatgtccgtgtcagtcggggagagggtcaccttgagctgcagggccagtgacattgtgggcacttatgtttcctggtatcaacagaaaccagagcagtctcctaaattgctgatatatggggcatccaaccggtacactggggtccccgatcgcttcacaggcagtagatctgcaacagatttcagtctgaccatcagtaatgtgcaggctgaagaccttgcagattatctctgtggacagagttacgactctccgtacacgttcggaggggggaccaagctggaaataaaa

SEQ ID NO: 341 - DNA, 351 bp, Artificial Sequence
  Description of Artificial Sequence Synthetic polynucleotide
caggtgcaactgcagcagtctgggcctcagctagttaggcctggggcttcagtgaagatatcctgcaaggcttctggttactcattcaccaactactggatgcactggatgaagcagaggcctggacaaggtcttgaatggattggcatgattgatccttccgatagtgagactaggttaaatcagcagttcaaggacaaggccacattgactgttgacaaatcctccagcacagcctacatgcaactcagcagcccgacatctgaggactctgcggtctattattgtgcaagattagggaggtattattttgacttctggggccaaggcaccactctcacagtctcctca

SEQ ID NO: 342 - DNA, 321 bp, Artificial Sequence
  Description of Artificial Sequence Synthetic polynucleotide
aacattgtaatgacccaatctcccaaatccatgtccgtgtcagtcggggagagggtcaccttgagctgcagggccagtgagattgtgggcacttatgtttcctggtatcaacagaaaccagagcagtctcctaaattgctgatatatggggcatccaaccggttcactggggtccccgatcgcttcacaggcagtagatctgcaacagatttcagtctgaccatcagtaatgtgcaggctgaagaccttgcagattatctctgtggacagagttacgactctccgtacacgttcggaggggggaccaagctggaaataaag

SEQ ID NO: 343 - DNA, 351 bp, Artificial Sequence
  Description of Artificial Sequence Synthetic polynucleotide
cagatgcaactgcagcagtctgggcctcaactggttaggcctggggcttcagtgaagatatcctgcaagacttctggttactcattcacccaccactggatacactggatgaagcagaggcctggacaaggtcttgagtggattggcatgattgatccctccgatagtgaaactagattaagtcagaagttcaaggacaaggccacattgactgtagacgcatcctccagcacagcctacatgcaactcaacagcccgacatctgaagactctgcgctctatttctgtgcaagattagggcggtactactttgactactggggccaaggaaccactctcacagtctcctca

SEQ ID NO: 344 - DNA, 321 bp, Artificial Sequence
  Description of Artificial Sequence Synthetic polynucleotide
aacattgtcatgacccagtctcccagatccatgtccatgtcagttggagagagggtcaccttgagctgcaaggccagtgagaatgtgggtacttatatatcctggtatcaacagaaaccagaccagtctcctaaactgctgatatacggggcatccaaccggtacactggggtccccgatcgcttcacaggcagtggatctggaacagatttcactctgaccatcagcactgtgcaggctgaagaccttgcagattatcactgtggagagagttatggtcatctgtacacgttcggaggggggaccaagctggaaataaaa

SEQ ID NO: 345 - DNA, 351 bp, Artificial Sequence
  Description of Artificial Sequence Synthetic polynucleotide
caggtgcaactgcagcagtctgggcctcaactggttaggcctggggcttcagtgaagatttcctgcaaggcttctggttactctttcaccaataactggatgcactggatgaaacagaggcctggacaaggtcttgaatggattggcatgattgatccttccgatagtgagaccaggttaaatcagcagttcagggacaaggccacattgactgttgacaaaacctccagcacagcctacatgcaactcagcagcccgacatctgaggactctgcggtctattactgtgcaagattagggcggtattattttgactactggggcctaggcaccactctcacagtctcctca

SEQ ID NO: 346 - DNA, 321 bp, Artificial Sequence
  Description of Artificial Sequence Synthetic polynucleotide
aatattgtaatgacccaatctcccaaatccatgtccgtgtcagtcggggagagggtcacaatgaactgcagggccagtgagattgtgggcacttatgtttcctggtatcaacaaaaaccagagcagtctcctaaattgctaatatacggggcattcaaccgctacactggggtccccgatcgcttcactggcagtagatctggaacagatttcagtctgaacatcagtaatgtgcaggctgaagaccttgcagattatctctgtggacagagttacgactctccgtacacgttcggaggggggaccaagctggaaataaaa

SEQ ID NO: 347 - DNA, 360 bp, Artificial Sequence
  Description of Artificial Sequence Synthetic polynucleotide
gaggttcagctgcagcagtctggggcagagtttgtgaagccaggggcctcagtcaagttgtcctgcacagcttctggcttcaatattgaagacacctatatgcactgggtgaagcagaggcctgaacagggcctggagtggattggaatgattgatcctgcgaatggtaaaactaaatatggcccgaggttccaggacaaggccactgtaacagcagacacatcctccaacacagccaacctgcagctcagcagcctgacatctgaggacactgccgtctattactgtgctgacggaattggttactacgtaggggctatggactactggggtcaaggaacctcagtcaccgtctcctca

SEQ ID NO: 348 - DNA, 321 bp, Artificial Sequence
  Description of Artificial Sequence Synthetic polynucleotide
gacatccagatgaaccagtctccatccagtctgtctgcatcccttggagacacaattaccatcacttgccatgccagtcagaacattaatgtttggttaagctggtaccagcagaaaccaggaaatattcctaaactattgatctataaggcttccaacttgcacacaggcgtcccatcaaggtttagtggcagtggatctggaacaggtttcacattaaccatcagcagcctgcagcctgaagacattgccacttactactgtcaacagggtcacagttatccgtacacgttcggaggggggaccaagctggaaataaaa

SEQ ID NO: 349 - protein, 117 aa, Artificial Sequence
  Description of Artificial Sequence Synthetic polypeptide
QVQLQQSGPQLVRPGASVKISCKASGYSFTNYWMHWMKQRPGQGLEWIGMIDPSDSETRLNQQFKDKATLAVDKSSSTAYMQLSSPTSEDSAVYYCARLGRYYFDYWGQGTTLTVSS

SEQ ID NO: 350 - protein, 107 aa, Artificial Sequence
  Description of Artificial Sequence Synthetic polypeptide
NIVMTQSPKSMYVSVGERVTLICRASEIVGTYVSWYQQKPEQSPKLLIYGASNRYTGVPDRFTGSRSATDFSLTISNVQAEDLADYLCGQSYDSPYTFGGGTKLEIK

SEQ ID NO: 351 - protein, 117 aa, Artificial Sequence
  Description of Artificial Sequence Synthetic polypeptide
QVQLQQSGPQLVRPGASVKISCKASGYSFTNHWMHWMKQRPGQGLEWIGMIDPSDSETRLNQQFKDKATLTVDKSSSTAYMQLSSPTSEDSAVFYCARLGRYYFDYWGQGTTLTVSS

SEQ ID NO: 352 - protein, 107 aa, Artificial Sequence
  Description of Artificial Sequence Synthetic polypeptide
NIVMTQSPKSMSVSVGERVTLSCRASDIVGTYVSWYQQKPEQSPKLLIYGASNRYTGVPDRFTGSRSATDFSLTISNVQAEDLADYLCGQSYDSPYTFGGGTKLEIK

SEQ ID NO: 353 - protein, 117 aa, Artificial Sequence
  Description of Artificial Sequence Synthetic polypeptide
QVQLQQSGPQLVRPGASVKISCKASGYSFTNYWMHWMKQRPGQGLEWIGMIDPSDSETRLNQQFKDKATLTVDKSSSTAYMQLSSPTSEDSAVYYCARLGRYYFDFWGQGTTLTVSS

SEQ ID NO: 354 - protein, 107 aa, Artificial Sequence
  Description of Artificial Sequence Synthetic polypeptide
NIVMTQSPKSMSVSVGERVTLSCRASEIVGTYVSWYQQKPEQSPKLLIYGASNRFTGVPDRFTGSRSATDFSLTISNVQAEDLADYLCGQSYDSPYTFGGGTKLEIK

SEQ ID NO: 355 - protein, 120 aa, Artificial Sequence
  Description of Artificial Sequence Synthetic polypeptide
EVQLQQSGAEFVKPGASVKLSCTASGFNIEDTYMHWVKQRPEQGLEWIGMIDPANGKTKYGPRFQDKATVTADTSSNTANLQLSSLTSEDTAVYYCADGIGYYVGAMDYWGQGTSVTVSS

SEQ ID NO: 356 - protein, 107 aa, Artificial Sequence
  Description of Artificial Sequence Synthetic polypeptide
DIQMNQSPSSLSASLGDTITITCHASQNINVWLSWYQQKPGNIPKLLIYKASNLHTGVPSRFSGSGSGTGFTLTISSLQPEDIATYYCQQGHSYPYTFGGGTKLEIK

SEQ ID NO: 357 - protein, 117 aa, Artificial Sequence
  Description of Artificial Sequence Synthetic polypeptide
QMQLQQSGPQLVRPGASVKISCKTSGYSFTHHWIHWMKQRPGQGLEWIGMIDPSDSETRLSQKFKDKATLTVDASSSTAYMQLNSPTSEDSALYFCARLGRYYFDYWGQGTTLTVSS

SEQ ID NO: 358 - protein, 107 aa, Artificial Sequence
  Description of Artificial Sequence Synthetic polypeptide
NIVMTQSPRSMSMSVGERVTLSCKASENVGTYISWYQQKPDQSPKLLIYGASNRYTGVPDRFTGSGSGTDFTLTISTVQAEDLADYHCGESYGHLYTFGGGTKLEIK

SEQ ID NO: 359 - protein, 117 aa, Artificial Sequence
  Description of Artificial Sequence Synthetic polypeptide
QVQLQQSGPQLVRPGASVKISCKASGYSFTNNWMHWMKQRPGQGLEWIGMIDPSDSETRLNQQFRDKATLTVDKTSSTAYMQLSSPTSEDSAVYYCARLGRYYFDYWGLGTTLTVSS

SEQ ID NO: 360 - protein, 107 aa, Artificial Sequence
  Description of Artificial Sequence Synthetic polypeptide
NIVMTQSPKSMSVSVGERVTMNCRASEIVGTYVSWYQQKPEQSPKLLIYGAFNRYTGVPDRFTGSRSGTDFSLNISNVQAEDLADYLCGQSYDSPYTFGGGTKLEIK

SEQ ID NO: 361 - protein, 117 aa, Artificial Sequence; identical to SEQ ID NO: 357
  Description of Artificial Sequence Synthetic polypeptide

SEQ ID NO: 362 - protein, 117 aa, Artificial Sequence
  Description of Artificial Sequence Synthetic polypeptide
QMQLQQSGPQLVRPGASVKISCKTSGYSFTHHWIHWMKQRPGQGLEWIGIIDPSDSETRLSQKFKDKATLTVDASSSTAYMQLNSPTSEDSALYFCARLGRYYFDYWGQGTTLTVSS

SEQ ID NO: 363 - protein, 117 aa, Artificial Sequence
  Description of Artificial Sequence Synthetic polypeptide
QMQLQQSGPQLVRPGASVKISCKTSGYSFTHHWIHWMKQRPGQGLEWIGMIEPSDSETRLSQKFKDKATLTVDASSSTAYMQLNSPTSEDSALYFCARLGRYYFDYWGQGTTLTVSS

SEQ ID NO: 364 - protein, 117 aa, Artificial Sequence
  Description of Artificial Sequence Synthetic polypeptide
QMQLQQSGPQLVRPGASVKISCKTSGYSFTHHWIHWMKQRPGQGLEWIGMISPSDSETRLSQKFKDKATLTVDASSSTAYMQLNSPTSEDSALYFCARLGRYYFDYWGQGTTLTVSS

SEQ ID NO: 365 - protein, 117 aa, Artificial Sequence
  Description of Artificial Sequence Synthetic polypeptide
QMQLQQSGPQLVRPGASVKISCKTSGYSFTHHWIHWMKQRPGQGLEWIGMIDASDSETRLSQKFKDKATLTVDASSSTAYMQLNSPTSEDSALYFCARLGRYYFDYWGQGTTLTVSS

SEQ ID NO: 366 - protein, 117 aa, Artificial Sequence
  Description of Artificial Sequence Synthetic polypeptide
QVQLVQSGAEVKKPGSSVKVSCKASGYSFTHHWIHWVRQAPGQGLEWMGMIDPSDSETRLSQKFKDRVTITADKSTSTAYMELSSLRSEDTAVYYCARLGRYYFDYWGQGTTVTVSS

SEQ ID NO: 367 - protein, 117 aa, Artificial Sequence
  Description of Artificial Sequence Synthetic polypeptide
EVQLVQSGAEVKKPGSSVKVSCKASGYSFTHHWIHWMRQAPGQGLEWIGMIDPSDSETRLSQKFKDRVTITADKSTSTAYMELSSLRSEDTAVYFCARLGRYYFDYWGQGTTVTVSS

SEQ ID NO: 368 - protein, 117 aa, Artificial Sequence
  Description of Artificial Sequence Synthetic polypeptide
EVQLVQSGAEVKKPGSSVKVSCKASGYSFTHHWIHWMRQAPGQGLEWIGMIDPSDSETRLSQKFKDRATLTVDKSTSTAYMELSSLRSEDTAVYFCARLGRYYFDYWGQGTTVTVSS

SEQ ID NO: 369 - protein, 117 aa, Artificial Sequence
  Description of Artificial Sequence Synthetic polypeptide
EVQLVQSGAEVKKPGSSVKVSCKTSGYSFTHHWIHWMKQAPGQGLEWIGMIDPSDSETRLSQKFKDKATLTVDKSTSTAYMELSSLRSEDTAVYFCARLGRYYFDYWGQGTTVTVSS

SEQ ID NO: 370 - protein, 117 aa, Artificial Sequence
  Description of Artificial Sequence Synthetic polypeptide
EVQLVQSGAEVKKPGSSVKVSCKASGYSFTHHWIHWVRQAPGQGLEWMGMIDPSDSETRLSQKFKDRVTITADKSTSTAYMELSSLRSEDTAVYYCARLGRYYFDYWGQGTTVTVSS

SEQ ID NO: 371 - protein, 117 aa, Artificial Sequence
  Description of Artificial Sequence Synthetic polypeptide
EVQLVQSGAEVKKPGSSVKVSCKASGYSFTHHWIHWVRQAPGQGLEWMGMIEPSDSETRLSQKFKDRVTITADKSTSTAYMELSSLRSEDTAVYYCARLGRYYFDYWGQGTTVTVSS

SEQ ID NO: 372 - protein, 117 aa, Artificial Sequence
  Description of Artificial Sequence Synthetic polypeptide
EVQLVQSGAEVKKPGSSVKVSCKASGYSFTHHWIHWVRQAPGQGLEWMGMISPSDSETRLSQKFKDRVTITADKSTSTAYMELSSLRSEDTAVYYCARLGRYYFDYWGQGTTVTVSS

SEQ ID NO: 373 - protein, 117 aa, Artificial Sequence
  Description of Artificial Sequence Synthetic polypeptide
EVQLVQSGAEVKKPGSSVKVSCKASGYSFTHHWIHWVRQAPGQGLEWMGMIDASDSETRLSQKFKDRVTITADKSTSTAYMELSSLRSEDTAVYYCARLGRYYFDYWGQGTTVTVSS

SEQ ID NO: 374 - protein, 107 aa, Artificial Sequence; identical to SEQ ID NO: 358
  Description of Artificial Sequence Synthetic polypeptide

SEQ ID NO: 375 - protein, 107 aa, Artificial Sequence
  Description of Artificial Sequence Synthetic polypeptide
EIVLTQSPATLSLSPGERATLSCKASENVGTYISWYQQKPGQAPRLLIYGASNRYTGIPARFSGSGSGTDFTLTISSLEPEDFAVYYCGESYGHLYTFGGGTKVEIK

SEQ ID NO: 376 - protein, 107 aa, Artificial Sequence
  Description of Artificial Sequence Synthetic polypeptide
EIVLTQSPATLSLSPGERATLSCRASENVGTYISWYQQKPGQAPRLLIYGASNRYTGIPARFSGSGSGTDFTLTISSLEPEDFAVYYCGESYGHLYTFGGGTKVEIK

SEQ ID NO: 377 - protein, 107 aa, Artificial Sequence; identical to SEQ ID NO: 376
  Description of Artificial Sequence Synthetic polypeptide

SEQ ID NO: 378 - protein, 107 aa, Artificial Sequence
  Description of Artificial Sequence Synthetic polypeptide
EIVLTQSPATLSLSPGERATLSCRASENVGTYISWYQQKPGQAPRLLIYGASNRYTGVPARFSGSGSGTDFTLTISSLEPEDFAVYHCGESYGHLYTFGGGTKVEIK

SEQ ID NO: 379 - protein, 107 aa, Artificial Sequence
  Description of Artificial Sequence Synthetic polypeptide
EIVLTQSPATLSLSPGERVTLSCRASENVGTYISWYQQKPGQAPRLLIYGASNRYTGVPARFSGSGSGTDFTLTISSVEPEDFAVYHCGESYGHLYTFGGGTKLEIK

SEQ ID NO: 380 - protein, 120 aa, Artificial Sequence; identical to SEQ ID NO: 355
  Description of Artificial Sequence Synthetic polypeptide

SEQ ID NO: 381 - protein, 120 aa, Artificial Sequence
  Description of Artificial Sequence Synthetic polypeptide
EVQLVQSGAEVKKPGSSVKVSCKASGFNIEDTYMHWVRQAPGQGLEWMGMIDPANGKTKYGPRFQDRVTITADKSTSTAYMELSSLRSEDTAVYYCARGIGYYVGAMDYWGQGTTVTVSS

SEQ ID NO: 382 - protein, 120 aa, Artificial Sequence
  Description of Artificial Sequence Synthetic polypeptide
EVQLVQSGAEVKKPGSSVKVSCKASGFNIEDTYMHWVRQAPGQGLEWIGMIDPANGKTKYGPRFQDRVTITADKSTSTAYMELSSLRSEDTAVYYCADGIGYYVGAMDYWGQGTTVTVSS

SEQ ID NO: 383 - protein, 120 aa, Artificial Sequence
  Description of Artificial Sequence Synthetic polypeptide
EVQLVQSGAEVKKPGSSVKVSCKASGFNIEDTYMHWVRQAPGQGLEWIGMIDPANGKTKYGPRFQDRATVTADKSTSTAYMELSSLRSEDTAVYYCADGIGYYVGAMDYWGQGTTVTVSS

SEQ ID NO: 384 - protein, 107 aa, Artificial Sequence; identical to SEQ ID NO: 356
  Description of Artificial Sequence Synthetic polypeptide

SEQ ID NO: 385 - protein, 107 aa, Artificial Sequence
  Description of Artificial Sequence Synthetic polypeptide
DIQMTQSPSSLSASVGDRVTITCHASQNINVWLSWYQQKPGKAPKLLIYKASNLHTGVPSRFSGSGSGTDFTLTISSLQPEDFATYYCQQGHSYPYTFGGGTKVEIK

SEQ ID NO: 386 - protein, 107 aa, Artificial Sequence
  Description of Artificial Sequence Synthetic polypeptide
DIQMTQSPSSLSASVGDRVTITCRASQNINVWLSWYQQKPGKAPKLLIYKASNLHTGVPSRFSGSGSGTDFTLTISSLQPEDFATYYCQQGHSYPYTFGGGTKVEIK

SEQ ID NO: 387 - protein, 107 aa, Artificial Sequence
  Description of Artificial Sequence Synthetic polypeptide
DIQMTQSPSSLSASVGDRITITCRASQNINVWLSWYQQKPGKAPKLLIYKASNLHTGVPSRFSGSGSGTDFTLTISSLQPEDFATYYCQQGHSYPYTFGGGTKLEIK

SEQ ID NO: 388 - protein, 117 aa, Artificial Sequence; identical to SEQ ID NO: 351
  Description of Artificial Sequence Synthetic polypeptide

SEQ ID NO: 389 - protein, 117 aa, Artificial Sequence
  Description of Artificial Sequence Synthetic polypeptide
QVQLVQSGAEVKKPGSSVKVSCKASGYSFTNHWMHWVRQAPGQGLEWMGMIDPSDSETRLNQQFKDRVTITADKSTSTAYMELSSLRSEDTAVYYCARLGRYYFDYWGQGTTVTVSS

SEQ ID NO: 390 - protein, 117 aa, Artificial Sequence
  Description of Artificial Sequence Synthetic polypeptide
EVQLVQSGAEVKKPGSSVKVSCKASGYSFTNHWMHWMRQAPGQGLEWIGMIDPSDSETRLNQQFKDRVTITADKSTSTAYMELSSLRSEDTAVFYCARLGRYYFDYWGQGTTVTVSS

SEQ ID NO: 391 - protein, 117 aa, Artificial Sequence
  Description of Artificial Sequence Synthetic polypeptide
EVQLVQSGAEVKKPGSSVKVSCKASGYSFTNHWMHWMRQAPGQGLEWIGMIDPSDSETRLNQQFKDRATLTVDKSTSTAYMELSSLRSEDTAVFYCARLGRYYFDYWGQGTTVTVSS

SEQ ID NO: 392 - protein, 117 aa, Artificial Sequence
  Description of Artificial Sequence Synthetic polypeptide
EVQLVQSGAEVKKPGSSVKVSCKASGYSFTNHWMHWMKQAPGQGLEWIGMIDPSDSETRLNQQFKDKATLTVDKSTSTAYMELSSLRSEDTAVYYCARLGRYYFDYWGQGTTVTVSS

SEQ ID NO: 393 - protein, 107 aa, Artificial Sequence; identical to SEQ ID NO: 352
  Description of Artificial Sequence Synthetic polypeptide

SEQ ID NO: 394 - protein, 107 aa, Artificial Sequence
  Description of Artificial Sequence Synthetic polypeptide
EIVMTQSPATLSVSPGERATLSCRASDIVGTYVSWYQQKPGQAPRLLIYGASNRYTGIPARFSGSGSGTEFTLTISSLQSEDFAVYYCGQSYDSPYTFGGGTKVEIK

SEQ ID NO: 395 - protein, 107 aa, Artificial Sequence
  Description of Artificial Sequence Synthetic polypeptide
EIVMTQSPATLSVSPGERATLSCRASDIVGTYVSWYQQKPGQAPRLLIYGASNRYTGVPARFSGSGSGTEFTLTISSLQSEDFAVYLCGQSYDSPYTFGGGTKVEIK

SEQ ID NO: 396 - protein, 107 aa, Artificial Sequence
  Description of Artificial Sequence Synthetic polypeptide
EIVMTQSPATLSVSPGERVTLSCRASDIVGTYVSWYQQKPGQAPRLLIYGASNRYTGVPARFSGSGSGTEFTLTISSVQSEDFAVYLCGQSYDSPYTFGGGTKLEIK

SEQ ID NO: 397 - protein, 447 aa, Artificial Sequence
  Description of Artificial Sequence Synthetic polypeptide
QMQLQQSGPQLVRPGASVKISCKTSGYSFTHHWIHWMKQRPGQGLEWIGMIDPSDSETRLSQKFKDKATLTVDASSSTAYMQLNSPTSEDSALYFCARLGRYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 398 - protein, 214 aa, Artificial Sequence
  Description of Artificial Sequence Synthetic polypeptide
NIVMTQSPRSMSMSVGERVTLSCKASENVGTYISWYQQKPDQSPKLLIYGASNRYTGVPDRFTGSGSGTDFTLTISTVQAEDLADYHCGESYGHLYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 399 - protein, 447 aa, Artificial Sequence
  Description of Artificial Sequence Synthetic polypeptide
EVQLVQSGAEVKKPGSSVKVSCKASGYSFTHHWIHWVRQAPGQGLEWMGMIDASDSETRLSQKFKDRVTITADKSTSTAYMELSSLRSEDTAVYYCARLGRYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 400 - protein, 443 aa, Artificial Sequence
  Description of Artificial Sequence Synthetic polypeptide
EVQLVQSGAEVKKPGSSVKVSCKASGYSFTHHWIHWVRQAPGQGLEWMGMIDASDSETRLSQKFKDRVTITADKSTSTAYMELSSLRSEDTAVYYCARLGRYYFDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 401 - protein, 444 aa, Artificial Sequence
  Description of Artificial Sequence Synthetic polypeptide
EVQLVQSGAEVKKPGSSVKVSCKASGYSFTHHWIHWVRQAPGQGLEWMGMIDASDSETRLSQKFKDRVTITADKSTSTAYMELSSLRSEDTAVYYCARLGRYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 402 - protein, 214 aa, Artificial Sequence
  Description of Artificial Sequence Synthetic polypeptide
EIVLTQSPATLSLSPGERATLSCRASENVGTYISWYQQKPGQAPRLLIYGASNRYTGIPARFSGSGSGTDFTLTISSLEPEDFAVYYCGESYGHLYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 403 - protein, 444 aa, Artificial Sequence
  Description of Artificial Sequence Synthetic polypeptide
EVQLVQSGAEVKKPGSSVKVSCKASGYSFTHHWIHWVRQAPGQGLEWMGMIDASDSETRLSQKFKDRVTITADKSTSTAYMELSSLRSEDTAVYYCARLGRYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 404 - protein, 447 aa, Artificial Sequence
  Description of Artificial Sequence Synthetic polypeptide
QVQLQQSGPQLVRPGASVKISCKASGYSFTNHWMHWMKQRPGQGLEWIGMIDPSDSETRLNQQFKDKATLTVDKSSSTAYMQLSSPTSEDSAVFYCARLGRYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 405 - protein, 214 aa, Artificial Sequence
  Description of Artificial Sequence Synthetic polypeptide
NIVMTQSPKSMSVSVGERVTLSCRASDIVGTYVSWYQQKPEQSPKLLIYGASNRYTGVPDRFTGSRSATDFSLTISNVQAEDLADYLCGQSYDSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 406 - protein, 450 aa, Artificial Sequence
  Description of Artificial Sequence Synthetic polypeptide
EVQLQQSGAEFVKPGASVKLSCTASGFNIEDTYMHWVKQRPEQGLEWIGMIDPANGKTKYGPRFQDKATVTADTSSNTANLQLSSLTSEDTAVYYCADGIGYYVGAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 407 - protein, 214 aa, Artificial Sequence
  Description of Artificial Sequence Synthetic polypeptide
DIQMNQSPSSLSASLGDTITITCHASQNINVWLSWYQQKPGNIPKLLIYKASNLHTGVPSRFSGSGSGTGFTLTISSLQPEDIATYYCQQGHSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 408 - protein, 330 aa, Homo sapiens [sequence omitted]

SEQ ID NO: 409 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 410 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 411 - protein, 327 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 412 - protein, 323 aa, Artificial Sequence
  Description of Artificial Sequence Synthetic polypeptide
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 413 - DNA, 1329 bp, Artificial Sequence
  Description of Artificial Sequence Synthetic polynucleotide
gaggtgcagctggtgcagtccggagctgaggtgaagaagccaggatccagcgtgaaggtgagctgcaaggctagcggctactctttcacccaccattggatccactgggtgaggcaggctcctggacagggactggagtggatgggcatgatcgacgcttccgatagcgagacaagactgtctcagaagtttaaggaccgcgtgaccatcacagccgataagtctacctccacagcttacatggagctgtcttccctgagatccgaggacaccgccgtgtactattgtgctaggctgggccggtactatttcgattattggggccagggcaccacagtgacagtgagctctgcctccaccaagggacctagcgtgtttcccctggctccttgcagccggtctacatccgagagcaccgccgctctgggatgtctggtgaaggattatttccctgagccagtgacagtgtcttggaactccggcgccctgacaagcggagtgcacacctttccagctgtgctgcagtcttccggcctgtattctctgagctctgtggtgaccgtgccttccagcaatttcggcacccagacatacacctgcaacgtggaccataagccatccaatacaaaggtggataagaccgtggagagaaagtgctgcgtggagtgcccaccttgtcctgctccaccagtggctggaccaagcgtgttcctgtttcctccaaagcccaaggacacactgatgatctcccgcacacctgaggtgacctgcgtggtggtggacgtgagccacgaggatcccgaggtgcagtttaactggtacgtggatggcgtggaggtgcataatgctaagaccaagcctagggaggagcagttcaactctacatttcgggtggtgtccgtgctgaccgtggtgcaccaggactggctgaacggcaaggagtacaagtgcaaggtgtctaataagggcctgcccgctcctatcgagaagacaatctccaagaccaagggccagccaagagagccccaggtgtataccctgccccctagccgcgaggagatgacaaagaaccaggtgtctctgacctgtctggtgaagggcttctacccatctgacatcgccgtggagtgggagtccaatggccagcccgagaacaattataagaccacaccacccatgctggacagcgatggctctttctttctgtacagcaagctgacagtggataagtctaggtggcagcagggcaacgtgttttcttgctccgtgatgcatgaggctctgcacaatcattacacccagaagagcctgtctctgtcccctggcaag

SEQ ID NO: 414 - DNA, 642 bp, Artificial Sequence
  Description of Artificial Sequence Synthetic polynucleotide
gagatcgtgctgacccagtctccagccacactgtctctgtccccaggagagagggccaccctgagctgccgggcttctgagaacgtgggcacatacatctcctggtatcagcagaagccaggacaggctcctaggctgctgatctacggcgctagcaatagatataccggcatccctgctcgcttcagcggatctggatccggcacagactttaccctgacaatctccagcctggagccagaggatttcgccgtgtactattgtggcgagtcctacggccacctgtatacctttggcggcggcacaaaggtggagatcaagcgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 415 - DNA, 1329 bp, Artificial Sequence
  Description of Artificial Sequence Synthetic polynucleotide
gaggtgcagctggtgcagagcggagcagaggtgaagaagcctggcagctccgtgaaggtgtcctgcaaggcctccggctactctttcacacaccactggatccactgggtgcggcaggcaccaggacagggactggagtggatgggcatgatcgacgccagcgattccgagaccaggctgtcccagaagtttaaggaccgcgtgaccatcacagccgataagtctaccagcacagcctacatggagctgtctagcctgaggagcgaggacaccgccgtgtactattgtgcccggctgggcagatactatttcgattattggggccagggcaccacagtgacagtgtcctctgcctccaccaagggaccaagcgtgttcccactggcaccatgctcccgctctacaagcgagtccaccgccgccctgggatgtctggtgaaggactatttccctgagccagtgacagtgagctggaactccggcgccctgacatctggcgtgcacacctttcctgccgtgctgcagagctccggcctgtacagcctgtctagcgtggtgaccgtgccctcctctaatttcggcacccagacatatacctgcaacgtggaccacaagccttccaatacaaaggtggataagaccgtggagaggaagtgctgcgtggagtgcccaccttgtccagcaccaccagtggcaggccctagcgtgttcctgtttcctccaaagccaaaggacacactgatgatctctagaacacccgaggtgacctgcgtggtggtggacgtgagccacgaggatccagaggtgcagtttaactggtacgtggatggcgtggaggtgcacaatgccaagaccaagccccgggaggagcagttcaacagcaccttccgggtggtgtccgtgctgaccgtggtgcaccaggattggctgaacggcaaggagtataagtgcaaggtgtccaataagggcctgcccgcccctatcgagaagacaatctctaagaccaagggccagcctagggagccacaggtgtacaccctgccccctagccgcgaggagatgacaaagaaccaggtgtccctgacctgtctggtgaagggcttctatccttccgacatcgccgtggagtgggagtctaatggccagccagagaacaattacaagaccacaccacccatgctggactctgatggcagcttctttctgtattctaagctgacagtggataagagcagatggcagcagggcaacgtgttttcttgcagcgtgatgcacgaggccctgcacaatcactacacccagaagtccctgtctctgagccccggcaag

SEQ ID NO: 416 - DNA, 642 bp, Artificial Sequence
  Description of Artificial Sequence Synthetic polynucleotide
gagatcgtgctgacccagtcccctgccacactgagcctgtccccaggagagagggccaccctgtcttgcagagcaagcgagaacgtgggcacatacatctcttggtatcagcagaagccaggacaggcaccaaggctgctgatctacggagcaagcaataggtataccggcatccccgcacgcttctctggaagcggatccggcacagactttaccctgacaatcagctccctggagcctgaggatttcgccgtgtactattgcggcgagagctacggccacctgtatacctttggcggcggcacaaaggtggagatcaagaggaccgtggcagcaccaagcgtgttcatctttcccccttccgacgagcagctgaagtccggcaccgcctctgtggtgtgcctgctgaacaatttctaccccagagaggccaaggtgcagtggaaggtggataacgccctgcagtctggcaatagccaggagtccgtgaccgagcaggactctaaggatagcacatattccctgtctagcaccctgacactgtccaaggccgactacgagaagcacaaggtgtatgcatgcgaggtgacccaccagggactgtcctctcctgtgacaaagtcttttaacagaggcgagtgt

SEQ ID NO: 417 - DNA, 1329 bp, Artificial Sequence
  Description of Artificial Sequence Synthetic polynucleotide
gaggtgcagctggtgcagagcggagcagaggtgaagaagcctggcagctccgtgaaggtgtcctgcaaggcctccggctactctttcacacaccactggatccactgggtgcggcaggcaccaggacagggactggagtggatgggcatgatcgacgccagcgattccgagaccaggctgtcccagaagtttaaggaccgcgtgaccatcacagccgataagtctaccagcacagcctacatggagctgtctagcctgaggagcgaggacaccgccgtgtactattgtgcccggctgggcagatactatttcgattattggggccagggcaccacagtgacagtgtcctctgcctccaccaagggcccctctgtgtttccactggccccctgctccaggtctacaagcgagtccaccgcagcactgggatgtctggtgaaggactatttccctgagccagtgacagtgagctggaactccggcgccctgacatctggcgtgcacacctttcctgccgtgctgcagagctccggcctgtacagcctgtctagcgtggtgaccgtgccctcctctaatttcggcacccagacatatacctgcaacgtggaccacaagccttccaatacaaaggtggataagaccgtggagcggaagtgctgtgtggagtgcccaccttgtccagcaccaccagtggcaggccctagcgtgttcctgtttcctccaaagccaaaggacacactgatgatctctagaacacccgaggtgacctgtgtggtggtggacgtgagccacgaggatccagaggtgcagtttaactggtacgtggatggcgtggaggtgcacaatgccaagaccaagccccgggaggagcagttcaacagcaccttccgggtggtgtccgtgctgaccgtggtgcaccaggattggctgaacggcaaggagtataagtgcaaggtgtccaataagggcctgcccgcccctatcgagaagacaatctctaagaccaagggccagcctagggagccacaggtgtacaccctgccccctagccgcgaggagatgacaaagaaccaggtgtccctgacctgtctggtgaagggcttctatccttccgacatcgccgtggagtgggagtctaatggccagccagagaacaattacaagaccacaccacccatgctggactctgatggcagcttctttctgtattctaagctgacagtggataagagcagatggcagcagggcaacgtgttttcttgcagcgtgatgcacgaggccctgcacaatcactacacccagaagtccctgtctctgagccccggcaag

SEQ ID NO: 418 - DNA, 642 bp, Artificial Sequence
  Description of Artificial Sequence Synthetic polynucleotide
gagatcgtgctgacccagtcccctgcaacactgagcctgtccccaggagagagggcaaccctgtcttgcagagcaagcgagaacgtgggcacatacatctcttggtatcagcagaagccaggacaggcaccaaggctgctgatctacggagcaagcaataggtataccggcatccccgcacgcttctctggaagcggatccggcacagactttaccctgacaatcagctccctggagcctgaggatttcgccgtgtactattgcggcgagagctacggccacctgtatacctttggcggcggcacaaaggtggagatcaagaggaccgtggcagcaccaagcgtgttcatctttcccccttccgacgagcagctgaagtccggcaccgcctctgtggtgtgtctgctgaacaatttctaccccagagaggccaaggtgcagtggaaggtggataacgccctgcagtctggcaatagccaggagtccgtgaccgagcaggactctaaggatagcacatattccctgtctagcaccctgacactgtccaaggccgactacgagaagcacaaggtgtatgcatgcgaggtgacccaccagggactgtcctctcctgtgacaaagtcttttaacagaggcgagtgt

SEQ ID NO: 419 - DNA, 1332 bp, Artificial Sequence
  Description of Artificial Sequence Synthetic polynucleotide
gaggtgcagctggtgcagtccggagctgaggtgaagaagccaggatccagcgtgaaggtgagctgcaaggctagcggctactctttcacccaccattggatccactgggtgaggcaggctcctggacagggactggagtggatgggcatgatcgacgcttccgatagcgagacaagactgtctcagaagtttaaggaccgcgtgaccatcacagccgataagtctacctccacagcttacatggagctgtcttccctgagatccgaggacaccgccgtgtactattgtgctaggctgggccggtactatttcgattattggggccagggcaccacagtgacagtgagctctgccagcacaaagggcccttccgtgttcccactggctccctgctccagaagcacatctgagtccaccgccgctctgggctgtctggtgaaggactacttccctgagccagtgaccgtgtcctggaacagcggcgccctgacatctggcgtgcacacctttccagctgtgctgcagtccagcggcctgtactccctgtcttccgtggtgacagtgcccagctcttccctgggcaccaagacatatacctgcaacgtggaccataagccttccaataccaaggtggataagagggtggagagcaagtacggaccaccttgcccaccatgtccagctcctgagtttgagggaggaccatccgtgttcctgtttcctccaaagcctaaggacaccctgatgatcagccggacacctgaggtgacctgcgtggtggtggacgtgtctcaggaggatccagaggtgcagttcaactggtacgtggatggcgtggaggtgcacaatgctaagaccaagccaagagaggagcagtttaattccacataccgcgtggtgagcgtgctgaccgtgctgcatcaggattggctgaacggcaaggagtataagtgcaaggtgtccaataagggcctgcccagctctatcgagaagacaatcagcaaggctaagggacagcctagggagccacaggtgtacaccctgcccccttctcaggaggagatgacaaagaaccaggtgtccctgacctgtctggtgaagggcttctatccaagcgacatcgctgtggagtgggagtctaatggccagcccgagaacaattacaagaccacaccacccgtgctggactctgatggctccttctttctgtattctaggctgacagtggataagtcccggtggcaggagggcaacgtgtttagctgctctgtgatgcacgaggccctgcacaatcattatacccagaagtccctgagcctgtctctgggcaag

SEQ ID NO: 420 - DNA, 1332 bp, Artificial Sequence
  Description of Artificial Sequence Synthetic polynucleotide
gaggtgcagctggtgcagtctggcgccgaggtgaagaagccaggcagctccgtgaaggtgtcctgcaaggcctccggctactctttcacacaccactggatccactgggtgcggcaggcaccaggacagggactggagtggatgggcatgatcgacgccagcgattccgagacccggctgagccagaagtttaaggacagagtgaccatcacagccgataagtctaccagcacagcctacatggagctgtctagcctgaggtccgaggacaccgccgtgtactattgtgcccggctgggcagatactatttcgattattggggccagggcaccacagtgacagtgtcctctgcctccaccaagggaccaagcgtgttcccactggcaccatgctcccgctctacaagcgagtccaccgccgccctgggatgtctggtgaaggactatttccctgagccagtgaccgtgagctggaactccggcgccctgacaagcggagtgcacacctttcctgccgtgctgcagagctccggcctgtactccctgtctagcgtggtgacagtgccctcctctagcctgggcaccaagacatatacctgcaacgtggaccacaagcctagcaataccaaggtggataagcgggtggagtccaagtacggaccaccttgcccaccatgtccagcacctgagttcgagggaggaccaagcgtgttcctgtttcctccaaagcctaaggacacactgatgatctccagaacacctgaggtgacctgcgtggtggtggacgtgtctcaggaggatccagaggtgcagttcaactggtacgtggatggcgtggaggtgcacaatgccaagaccaagcctagggaggagcagtttaatagcacataccgcgtggtgtccgtgctgaccgtgctgcaccaggattggctgaacggcaaggagtataagtgcaaggtgagcaataagggcctgccatcctctatcgagaagacaatctccaaggccaagggccagcctagagagccacaggtgtacaccctgcccccttctcaggaggagatgacaaagaaccaggtgagcctgacctgtctggtgaagggcttctatccatccgacatcgccgtggagtgggagtctaatggccagcccgagaacaattacaagaccacaccacccgtgctggactctgatggcagcttctttctgtattctaggctgacagtggataagagccgctggcaggagggcaacgtgttttcttgcagcgtgatgcacgaggccctgcacaatcactacacccagaagtccctgtctctgagcctgggcaag

SEQ ID NO: 421 - DNA, 1332 bp, Artificial Sequence
  Description of Artificial Sequence Synthetic polynucleotide
gaggtgcagctggtgcagtctggcgccgaggtgaagaagccaggcagctccgtgaaggtgtcctgcaaggcctccggctactctttcacacaccactggatccactgggtgcggcaggcaccaggacagggactggagtggatgggcatgatcgacgccagcgattccgagacccggctgagccagaagtttaaggacagagtgaccatcacagccgataagtctaccagcacagcctacatggagctgtctagcctgaggtccgaggacaccgccgtgtactattgtgcccggctgggcagatactatttcgattattggggccagggcaccacagtgacagtgtcctctgcctccaccaagggcccctctgtgtttccactggccccctgctccaggtctacaagcgagtccaccgcagcactgggatgtctggtgaaggactatttccctgagccagtgaccgtgagctggaactccggagcactgacaagcggagtgcacacctttcctgccgtgctgcagagctccggcctgtactccctgtctagcgtggtgacagtgccctcctctagcctgggcaccaagacatatacctgcaacgtggaccacaagcctagcaataccaaggtggataagcgggtggagtccaagtacggaccaccttgcccaccatgtccagcacctgagttcgagggaggaccaagcgtgttcctgtttcctccaaagcctaaggacacactgatgatctccagaacacctgaggtgacctgtgtggtggtggacgtgtctcaggaggatccagaggtgcagttcaactggtacgtggatggcgtggaggtgcacaatgccaagaccaagcctagggaggagcagtttaatagcacataccgcgtggtgtccgtgctgaccgtgctgcaccaggattggctgaacggcaaggagtataagtgcaaggtgagcaataagggcctgccatcctctatcgagaagacaatctccaaggccaagggccagcctagagagccacaggtgtacaccctgcccccttctcaggaggagatgacaaagaaccaggtgagcctgacctgtctggtgaagggcttctatccatccgacatcgccgtggagtgggagtctaatggccagcccgagaacaattacaagaccacaccacccgtgctggactctgatggcagcttctttctgtattctaggctgacagtggataagagccgctggcaggagggcaacgtgttttcttgcagcgtgatgcacgaggccctgcacaatcactacacccagaagtccctgtctctgagcctgggcaag

(19) United States
(12) Patent Application Publication
(10) Pub. No.: US 2021/0070855 A1
(43) Pub. Date: Mar. 11, 2021

(54) ANTI-CD47 ANTIBODIES THAT DO NOT CAUSE SIGNIFICANT RED BLOOD CELL AGLLUTINATION

(71) Applicant: NANJING LEGEND BIOTECH CO., LTD., Jiangsu (CN)

(72) Inventors: Tao Zhao, Jiangsu (CN); Huihui Zhang, Nanjing (CN); Shuai Yang, Nanjing (CN); Yun Zhang, Nanjing (CN); Chuan-Chu Chou, Westfield, NJ (US); Shu Wu, Nanjing (CN)

(21) Appl. No.: 16/964,828

(22) PCT Filed: Jan. 24, 2019
(86) PCT No.: PCT/CN2019/072929

§ 371 Date: Jul. 24, 2020

(30) Foreign Application Priority Data
Jan. 24, 2018 (CN) PCT/CN2018/074055

(51) Int. Cl. C07K 16/28; A61P 35/00

(52) U.S. Cl. CPC C07K 16/2803; A61P 35/00; C07K 2317/24; C07K 2317/33; C07K 2317/524; C07K 2319/30; C07K 2317/73; C07K 2317/76; C07K 2317/92; C07K 2317/94; C07K 2317/565; C07K 2317/71

(57) ABSTRACT

Provided are antibodies including monoclonal, human, primate, rodent, mammalian, chimeric, humanized and CDR-grafted antibodies, and antigen binding fragments and antigen binding derivatives thereof. These antibodies bind to CD47 protein, particularly human CD47, modulate, e.g., inhibit, block, antagonize, neutralize or otherwise interfere with CD47 expression, activity and/or signaling, including inhibiting CD47 and SIRPa interaction; do not cause a significant level of hemagglutination of human red blood cells. These antibodies may not enhance RBC phagocytosis.

### CROSS REFERENCE TO RELATED APPLICATIONS

This application claims priority benefits of International Patent Applications No. PCT/CN2018/074055 filed on Jan. 24, 2018, the contents of which are incorporated herein by reference in their entirety.

### INCORPORATION OF SEQUENCE LISTING

The instant application contains a Sequence Listing, which has been submitted electronically in ASCII format and is hereby incorporated by reference in its entirety. Said ASCII copy, created on Jan. 23, 2018, is named 5200-002P1_SL.txt and is 223,367 bytes in size.

### TECHNICAL FIELD

This disclosure relates to the field of immunobiology and diseases, including cancer.

### BACKGROUND

CD47 (Cluster of Differentiation 47), also known as integrin associated protein (IAP), is a 50-kDa membrane protein with an amino-terminal immunoglobulin domain and a carboxyl-terminal multiply membrane-spanning region. It interacts with multiple ligands, including, without limitation, single-regulatory protein alpha (SIRPα), SIRPγ, integrins and thrombospondin-1 (TSP-1). SIRPα is expressed primarily on myeloid cells, including macrophages, myeloid dendritic cells (DCs), granulocytes, mast cells, and their precursors, including hematopoietic stem cells. CD47/SIRPα interaction transmits a “don't eat me” signal, preventing autologous phagocytosis.

Analysis of patient tumor and matched adjacent normal (non-tumor) tissue revealed that CD47 protein is overexpressed on cancer cells, which efficiently helps them to suppress phagocytic innate immune surveillance and elimination. Blocking the CD47-SIRPα interaction with anti-CD47 antibodies has been shown effective in inducing the phagocytosis of tumor cells in vitro and inhibiting the growth of the various hematological and solid tumors in vivo. Therefore, CD47 is a validated target for cancer therapies and appropriate antagonists of it are needed to make human therapeutics.

### SUMMARY

This disclosure provides antibodies, including monoclonal, human, primate, rodent, mammalian, chimeric, humanized and CDR-grafted antibodies, and antigen binding fragments and antigen binding derivatives thereof, that recognize and bind to CD47 protein, particularly human CD47. The disclosed antibodies can modulate, e.g., inhibit, block, antagonize, neutralize or otherwise interfere with CD47 expression, activity and/or signaling, and these antibodies do not cause a significant level of hemagglutination of human red blood cells. The disclosed antibodies, fragments and derivatives thereof, can modulate, e.g., inhibit, block, antagonize, neutralize or otherwise interfere with the interaction between CD47 and SIRPα (signal-regulatory-protein α). The disclosed antibodies, fragments or derivatives thereof, may be referred to collectively as “anti-CD47 antibodies of the disclosure,” or “disclosed anti-CD47 antibodies,” “the disclosed antibody,” and the like.

The disclosed antibody, which may be isolated antibody, comprises at least one antibody-antigen binding site; the antibody binds human CD47; inhibits, blocks, antagonizes, neutralizes or otherwise interferes with CD47 expression, activity and/or signaling; and does not cause significant agglutination of cells.

In certain aspects, nucleic acid constructs and molecules, including vectors, are provided encoding the disclosed anti-CD47 antibodies.

In certain aspects, pharmaceutical compositions and kits comprising the disclosed anti-CD47 antibodies are provided.

In certain aspects, methods are disclosed using the disclosed anti-CD47 antibodies, pharmaceutical compositions, and kits, for treating, delaying the progression of, preventing relapse of, or alleviating a symptom of a cancer or other neoplastic condition; such as, for example, treating hematological malignancies and/or tumors, e.g., hematological malignancies and/or tumors. In certain embodiments, the disclosed CD47 antibodies are useful in treating CD47⁺ tumors. By way of non-limiting example, the anti-CD47 antibodies described herein are used to treat non-Hodgkin's lymphoma (NHL), acute lymphocytic leukemia (ALL), acute myeloid leukemia (AML), chronic lymphocytic leukemia (CLL), chronic myelogenous leukemia (CML), multiple myeloma (MM), breast cancer, ovarian cancer, head and neck cancer, bladder cancer, melanoma, colorectal cancer, pancreatic cancer, lung cancer, leiomyoma, leiomyosarcoma, glioma, glioblastoma. Solid tumors include, for example, breast tumors, ovarian tumors, lung tumors, pancreatic tumors, prostate tumors, melanoma tumors, colorectal tumors, lung tumors, head and neck tumors, bladder tumors, esophageal tumors, liver tumors, and kidney tumors.

Without limiting the disclosure, a number of embodiments of the disclosure are described below for purpose of illustration.

Item 1. An anti-CD47 antibody comprising at least one antibody-antigen binding site, said antibody binds human CD47, inhibits, blocks, antagonizes, neutralizes or otherwise interferes with CD47 expression, activity and/or signaling, and does not cause significant agglutination of cells, wherein said antibody is a human antibody, a chimeric antibody, a humanized antibody, a primatized antibody, a bi-specific antibody, a conjugated antibody, a Small Modular ImmunoPharmaceutical, a single chain antibody, a cameloid antibody, a CDR-grafted antibody, or an antigen-binding fragment or antigen binding functional variant thereof.

Item 2. The antibody of item 1, wherein said antibody does not cause hemagglutination of human red blood cells.

Item 3. The antibody of anyone of the preceding items, wherein said anti-CD47 antibody blocks the interaction between human CD47 and human signal-regulatory-protein α (SIRPα).

Item 4. The antibody of Item 3, wherein the antibody blocks at least 40%, at least 45%, at least 50%, at least 55%, at least 60%, at least 65%, at least 70%, at least 75%, at least 80%, at least 85%, at least 95%, or at least 99% of the interaction between CD47 and SIRPα as compared to the level of interaction between CD47 and SIRPα in the absence of the anti-CD47 antibody.

Item 5. The antibody of anyone of the preceding items, wherein less than a significant level of agglutination is a level of agglutination of cells in the presence of anti-CD47 antibody B6H12.

Item 6. The antibody of anyone of the preceding items, wherein the level of cell agglutination in the presence of said antibody is reduced by at least 5%, at least 10%, at least 20%, at least 30%, at least 40%, at least 50%, at least 60%, at least 70%, at least 80%, at least 90%, or at least 99% compared to the level of agglutination of cells in the presence of anti-CD47 antibody B6H12.

Item 7. The antibody of anyone of the preceding items, wherein the antibody does not cause a significant level of cell agglutination of cells at an antibody amount of between about 0.3 μg/ml to about 200 μg/ml.

Item 8. The antibody of anyone of items 1-6, wherein the antibody does not cause a significant level of agglutination of cells at an antibody concentration of between about 100 μg/ml and about 200 μg/ml.

Item 9. The antibody of anyone of the preceding items, wherein said antibody has potent anti-tumor activity.

Item 10. The antibody of item 9, wherein said potent anti-tumor activity is measured by the ability of macrophages to phagocytose tumor cells being increased by at least 5%, at least 10%, at least 20%, at least 30%, at least 40%, at least 50%, at least 60%, at least 70%, at least 80%, at least 90%, or at least 99% in the presence of said antibody, compared to the ability of macrophages to phagocytose tumor cells in the presence of anti-CD47 antibody B6H12.

Item 11. The antibody of anyone of the preceding items, wherein said antibody does not promote clumping of CD47 positive cell lines.

Item 12. The antibody of anyone of the preceding items, wherein said antibody comprises a variable heavy chain selected from SEQ ID NOs: 349, 351, 353, 355, 357, 359, 361-373, 380-383, 388-392 and a variable light chain selected from SEQ ID NOs:350, 352, 354, 356, 358, 360, 374-379, 384-387 and 393-396.

Item 13. The antibody of anyone of the preceding items, wherein said antibody comprises a variable heavy chain that is at least 90%, 91%, 92%, 93%, 94%, 95%, 96%, 97%, 98%, 99% or more identical to a sequence set forth in one of SEQ ID NOs: 349, 351, 353, 355, 357, 359, 361-373, 380-383, 388-392, and a variable light chain that is at least 90%, 91%, 92%, 93%, 94%, 95%, 96%, 97%, 98%, 99% or more identical to a sequence set forth in one of SEQ ID NOs: 350, 352, 354, 356, 358, 360, 374-379, 384-387 and 393-396.

Item 14. The antibody of anyone of one of items 1-2 or any one of items 5-13, wherein CD47 expression or activity in the presence of said antibody is decreased by at least 50%, 55%, 60%, 75%, 80%, 85% or 90% as compared to the level of CD47 expression or activity in the absence of said antibody.

Item 15. The antibody of item 14, wherein CD47 expression or activity in the presence of said antibody is decreased by at least 95%, 96%, 97%, 98%, 99% or 100% as compared to the level of CD47 expression or activity in the absence of said antibody.

Item 16. The antibody of anyone of the preceding items, wherein said antibody is an IgG isotype.

Item 17. The antibody of anyone of the preceding claims, wherein said antibody comprises a constant region modified at amino acid Asn297.

Item 18. The antibody of item 17, wherein said antibody comprises a constant region with an amino acid modification of N297A.

Item 19. The antibody of anyone of the preceding items, wherein said antibody comprises a constant region modified at amino acid Leu235 or Leu234.

Item 20. The antibody of item 19, wherein said constant region has an amino acid modification of Leu235Glu (L235E) or Leu235Ala (L235A), and/or an amino acid modification of Leu234Ala (L234A).

Item 21. The antibody of anyone of the preceding items, wherein said antibody comprises a human IgG₃ constant region that is modified at amino acid amino acid Arg435.

Item 22. The antibody of item 21, wherein said human IgG₃ constant region has an amino acid modification of Arg435His (R435H).

Item 23. The antibody of anyone of the preceding items, wherein said antibody comprises a human IgG₄ constant region that is modified within the hinge region to prevent or reduce strand exchange.

Item 24. The antibody of item 23, wherein said antibody comprises a human IgG₄ constant region with an amino acid modification of Ser228Pro (S228P).

Item 25. The antibody of item 23 or item 24, wherein said human IgG₄ constant region is further modified at amino acid 235.

Item 26. The antibody of item 25, wherein said human IgG₄ constant region has an amino acid modification of Leu235Glu (L235E).

Item 27. The antibody of anyone of the preceding items, wherein said antibody comprises a human IgG constant region modified to enhance FcRn binding, wherein said human IgG constant region has one or more amino acid modifications of Met252Tyr, Ser254Thr, Thr256Glu, Met428Leu or Asn434Ser (M252Y, S254T, T256E M428L, or N434S).

Item 28. The antibody of anyone of the preceding items, wherein said antibody comprises a human IgG constant region modified to alter antibody-dependent cellular cytotoxicity (ADCC) and/or complement-dependent cytotoxicity (CDC).

Item 29. The antibody of anyone of the preceding items, wherein said antibody comprises a human IgG constant region modified to induce heterodimerization, wherein said antibody has an amino acid modification of T366W or T366S and/or an amino acid modification of, L368A, or Y407V, S354C, or Y349C.

Item 30. The antibody of anyone of the preceding items, wherein said antibody is a humanized or human antibody having a variable heavy chain region (V_{H}) and/or variable light (V_{L}) chain region selected from the group consisting of: SEQ ID NOs: 366, 367, 368, 369, 373, 377, 378, 379, 381, 382, 383, 385, 386, 387, 399, 400, 401, 402, or 403.

Item 31. The antibody of anyone of the preceding items, wherein said antibody is a humanized or human antibody having a variable heavy chain region (V_{H}) and/or variable light (V_{L}) chain region that at least 90%, 91%, 92%, 93%, 94%, 95%, 96%, 97%, 98%, 99% or more identical to a sequence set forth in one of selected from the group consisting of: SEQ ID NOs: 366-373, 375, 377-379, 381-383, 385, 389-392, 394-396, 399-403

Item 32. The antibody of anyone of the preceding items, wherein said antibody is a humanized antibody.

Item 33. The antibody of anyone of the preceding claims, wherein said antibody is a human antibody.

Item 34. A vector comprising a nucleic acid encoding an antibody of any one of items 1-33.

Item 35. A vector comprising:

a nucleic acid encoding a heavy chain region (V_{H}) of an antibody and/or variable light (V_{L}) chain region of an antibody that is at least 90%, 91%, 92%, 93%, 94%, 95%, 96%, 97%, 98%, 99% or more (including 100%) identical to a sequence selected from the group consisting of: SEQ ID NOs: 337-348; 413-421.

Item 36. A prokaryotic cell, a yeast cell, a plant cell, or a mammalian cell line comprising a vector of item 34 or item 35, wherein said cell expresses the antibody of any one of items 1-33.

Item 37. A pharmaceutical composition comprising an antibody of any one of items 1-33 and a pharmaceutically acceptable excipient.

Item 38. A method of treating, delaying the progression of, preventing relapse of, or alleviating a symptom of a cancer or other neoplastic condition in a human patient with cancer or other neoplastic condition, comprising administering to said patient a therapeutically effective amount of an antibody comprising at least one antibody-antigen binding site, said antibody binds human CD47, inhibits, blocks, antagonizes, neutralizes or otherwise interferes with CD47 expression, activity and/or signaling, and does not cause significant agglutination of cells or administering to said patient a therapeutically effective amount of a pharmaceutical composition comprising said antibody and a pharmaceutical excipient.

Item 39. The method of item 38, wherein said antibody is an antibody according to any one of items 1-33.

Item 40. The method of item 38, wherein said pharmaceutical composition is a pharmaceutical composition of item 37.

Item 41. The method of any one of items 38-40, wherein said cancer or other neoplastic condition is a CD47⁺ tumor.

Item 42. The method of any one of items 38-41, wherein said cancer or other neoplastic condition is selected from the group consisting of non-Hodgkin's lymphoma (NHL), acute lymphocytic leukemia (ALL), acute myeloid leukemia (AML), chronic lymphocytic leukemia (CLL), chronic myelogenous leukemia (CML), and multiple myeloma (MM).

Item 43. The method of any one of items 38-41, wherein said cancer or other neoplastic condition is selected from the group consisting of breast cancer, ovarian cancer, head and neck cancer, bladder cancer, melanoma, colorectal cancer, pancreatic cancer, lung cancer, leiomyoma, leiomyosarcoma, glioma, glioblastoma, breast tumors, ovarian tumors, lung tumors, pancreatic tumors, prostate tumors, melanoma tumors, colorectal tumors, lung tumors, head and neck tumors, bladder tumors, esophageal tumors, liver tumors, and kidney tumors.

Item 44. The method of any one of items 38-41, wherein said cancer or other neoplastic condition is a hematological cancer.

Item 45. The method of item 44, wherein said hematological cancer is leukemia, lymphoma or myeloma.

Item 46. The method of item 44, wherein said hematological cancer is a leukemia selected from the group consisting of acute lymphocytic leukemia (ALL), acute myeloid leukemia (AML), chronic lymphocytic leukemia (CLL), chronic myelogenous leukemia (CML), Myeloproliferative disorder/neoplasm (MPDS), and myelodysplasia syndrome.

Item 47. The method of item 44, wherein said hematological cancer is a lymphoma selected from the group consisting of a Hodgkin's lymphoma, both indolent and aggressive non-Hodgkin's lymphoma, Burkitt's lymphoma, and follicular lymphoma (small cell and large cell).

Item 48. The method of item 44, wherein said hematological cancer is a myeloma selected from the group consisting of multiple myeloma (MM), giant cell myeloma, heavy-chain myeloma, and light chain or Bence-Jones myeloma.

Item 49. The method of any one of items 38-48, further comprising administering one or more additional agents to said patient.

Item 50. The method of item 49, wherein said additional agent is a therapeutic agent.

Item 51. The method of item 50, wherein said therapeutic agent is an anti-cancer agent.

Item 52. The antibody of anyone of the preceding items, wherein the said antibody comprising:
(a) a heavy chain variable domain (V_{H}) comprising
i. a heavy chain CDR1 comprising an amino acid sequence selected from the group consisting of SEQ ID NOs: 49, 51, 53, 55, 57, 59, 62-65, 86-87;
ii. a heavy chain CDR2 comprising an amino acid sequence selected from the group consisting of SEQ ID NOs: 145, 147, 149, 151, 153, 155, 158-161, 182-183; and
iii. a heavy chain CDR3 comprising an amino acid sequence selected from the group consisting of SEQ ID NOs: 241, 243, 245, 247, 249, 251, 254-257, 278-279 and
(b) a light chain variable domain (V_{L}) comprising, respectively,
i. a light chain CDR1 comprising an amino acid sequence selected from the group consisting of SEQ ID NOs: 50, 52, 54, 56, 58, 60, 76-79;
ii. a light chain CDR2 comprising an amino acid sequence selected from the group consisting of SEQ ID NOs: 146, 148, 150, 152, 154, 156, 172-175; and
iii. a light chain CDR3 comprising an amino acid sequence selected from the group consisting of SEQ ID NOs: 242, 244, 246, 248, 250, 252, 268-271.

In item 53. The antibody of item 52, wherein the said antibody comprise any one of the following:
(1) a V_{H} comprises the heavy chain CDR1, CDR2 and CDR3 sequences having the amino acid sequences of SEQ ID NOs: 49, 145 and 241, respectively, and a V_{L} comprises the light chain CDR1, CDR2 and CDR3 having the amino acid sequences of SEQ ID NOs: 50, 146 and 242, respectively;
(2) a V_{H} comprises the heavy chain CDR1, CDR2 and CDR3 sequences having the amino acid sequences of SEQ ID NOs: 51, 147 and 243, respectively, and a V_{L} comprises the light chain CDR1, CDR2 and CDR3 having the amino acid sequences of SEQ ID NOs: 52, 148 and 244, respectively;
(3) a V_{H} comprises the heavy chain CDR1, CDR2 and CDR3 sequences having the amino acid sequences of SEQ ID NOs: 53, 149 and 245, respectively, and a V_{L} comprises the light chain CDR1, CDR2 and CDR3 having the amino acid sequences of SEQ ID NOs: 54, 150 and 246, respectively;
(4) a V_{H} comprises the heavy chain CDR1, CDR2 and CDR3 sequences having the amino acid sequences of SEQ ID NOs: 55, 151 and 247, respectively, and a V_{L} comprises the light chain CDR1, CDR2 and CDR3 having the amino acid sequences of SEQ ID NOs: 56, 152 and 248, respectively;
(5) a V_{H} comprises the heavy chain CDR1, CDR2 and CDR3 sequences having the amino acid sequences of SEQ ID NOs: 57, 153 and 249, respectively, and a V_{L} comprises the light chain CDR1, CDR2 and CDR3 having the amino acid sequences of SEQ ID NOs: 58, 154 and 250, respectively;
(6) a V_{H} comprises the heavy chain CDR1, CDR2 and CDR3 sequences having the amino acid sequences of SEQ ID NOs: 59, 155 and 251, respectively, and a V_{L} comprises the light chain CDR1, CDR2 and CDR3 having the amino acid sequences of SEQ ID NOs: 60, 156 and 252, respectively;
(7) a V_{H} comprises the heavy chain CDR1, CDR2 and CDR3 sequences having the amino acid sequences of SEQ ID NOs:62, 158 and 254, respectively, and a V_{L} comprises the light chain CDR1, CDR2 and CDR3 having the amino acid sequences of SEQ ID NOs: 76, 172 and 268, respectively;
(8) a V_{H} comprises the heavy chain CDR1, CDR2 and CDR3 sequences having the amino acid sequences of SEQ ID NOs:63, 159 and 255, respectively, and a V_{L} comprises the light chain CDR1, CDR2 and CDR3 having the amino acid sequences of SEQ ID NOs: 77, 173 and 269, respectively;
(9) a V_{H} comprises the heavy chain CDR1, CDR2 and CDR3 sequences having the amino acid sequences of SEQ ID NOs:64, 160 and 256, respectively, and a V_{L} comprises the light chain CDR1, CDR2 and CDR3 having the amino acid sequences of SEQ ID NOs: 78, 174 and 270, respectively;
(10) a V_{H} comprises the heavy chain CDR1, CDR2 and CDR3 sequences having the amino acid sequences of SEQ ID NOs:65, 161 and 257, respectively, and a V_{L} comprises the light chain CDR1, CDR2 and CDR3 having the amino acid sequences of SEQ ID NOs: 79, 175 and 271, respectively;
(11) a V_{H} comprises the heavy chain CDR1, CDR2 and CDR3 sequences having the amino acid sequences of SEQ ID NOs:65, 161 and 257, respectively, and a V_{L} comprises the light chain CDR1, CDR2 and CDR3 having the amino acid sequences of SEQ ID NOs: 76, 172 and 268, respectively;
(12) a V_{H} comprises the heavy chain CDR1, CDR2 and CDR3 sequences having the amino acid sequences of SEQ ID NOs:86, 182 and 278, respectively, and a V_{L} comprises the light chain CDR1, CDR2 and CDR3 having the amino acid sequences of SEQ ID NOs: 56, 152 and 248, respectively; and
(13) a V_{H} comprises the heavy chain CDR1, CDR2 and CDR3 sequences having the amino acid sequences of SEQ ID NOs:87, 183 and 279, respectively, and a V_{L} comprises the light chain CDR1, CDR2 and CDR3 having the amino acid sequences of SEQ ID NOs: 56, 152 and 248, respectively.

Numerous other aspects are provided in accordance with these and other aspects of the invention. Other features and aspects of the present invention will become more fully apparent from the following detailed description and the appended claims.

### BRIEF DESCRIPTION OF THE DRAWINGS

FIG. 1A is a series of graphs depicting the binding of CD47 protein to some of the purified murine antibodies by ELISA. FIG. 1B is a series of graphs showing the binding of CD47 on Raji cells by antibodies (FIG. 1B). 2D3 was used as positive control.

FIG. 2 is a graph that shows RBC hemagglutination by purified murine antibodies (FIG. 2A and FIG. 2B). Commercial B6H12 antibody and 2D3 were used as positive and negative controls, respectively.

FIG. 3 is a series of graphs depicting the binding of CD47 on CHO-K1/huCD47 cell line by murine antibodies as assessed by flow cytometry. CHO-K1/huCD47 is an engineered cell line over-expressing human CD47 protein.

FIG. 4 is a series of graphs depicting the binding of CD47 on CHO-K1/cyno CD47 cell lines by murine CD47 antibodies as assessed by flow cytometry (FIG. 4A and FIG. 4B). CHO-K1/cyno CD47 is an engineered cell line over-expressing cynomolgus (cyno) CD47.

FIG. 5 is a series of graphs depicting the binding of CD47-his protein to purified murine CD47 antibodies as assessed by surface plasmon resonance (SPR).

FIG. 6 is a series of graphs depicting the capacity of murine CD47 antibodies to block SIRPα by flow cytometry using CHO-K1/huCD47 cell line. B6H12 was used as positive control.

FIG. 7 is a series of graphs depicting the ability of murine CD47 antibodies to promote phagocytosis of human tumor cell line CCRF-CEM by human monocyte derived macrophages (MDM) (FIG. 7A and FIG. 7B). CCRF-CEM cells were used as the CD47 target cell line in the experiments. B6H12 was used as positive control.

FIG. 8 is a series of graphs depicting the binding of CD47 on red blood cells by murine CD47 antibodies as assessed by flow cytometry. B6H12 was used as positive control.

FIG. 9 is a series of graphs depicting the ability of murine CD47 antibodies to promote phagocytosis of human red blood cells (RBC) by human monocyte derived macrophages (MDM). All murine antibodies showed obvious phagocytosis activity of RBC. B6H12 was used as positive control.

FIG. 10 is a graph that shows no RBC hemagglutination by chimeric antibodies.

FIG. 11 is a series of graphs depicting the ability of chimeric CD47 antibodies to promote phagocytosis of human tumor cell line CCRF-CEM by human monocyte derived macrophages (MDM).

FIG. 12 is a graph that shows no RBC hemagglutination by the IgG₁, IgG₂, and IgG₄PE isotypes of the humanized 108VH4.M4_VL1.M1.

FIG. 13 is a series of graphs depicting the binding of CD47-his protein to the IgG₁, IgG₂, and IgG₄PE isotypes of the humanized 108VH4.M4_VL1.M1 as assessed by SPR. Murine anti-CD47 antibody 108C10A6 was used as a positive control.

FIG. 14 is a series of graphs depicting the binding of CD47 on CHO-K1/huCD47 cell lines by the IgG₁, IgG₂, and IgG₄PE isotypes of the humanized 108VH4.M4_VL1.M1 as assessed by flow cytometry.

FIG. 15 is a series of graphs depicting the binding of the IgG₁, IgG₂, and IgG₄PE isotypes of the humanized 108VH4.M4_VL.M1 to RBC assessed by flow cytometry.

FIG. 16 is a series of graphs depicting the binding of the IgG₁, IgG₂, and IgG₄PE isotypes of the humanized 108VH4.M4_VL1.M1 to CHO-K1/cynoCD47 assessed by flow cytometry.

FIG. 17 is a series of graphs depicting the capacity of the IgG₁, IgG₂, and IgG₄PE isotypes of the humanized 108VH4.M4_VL1.M1 to block SIRPα by flow cytometry using CHO-K1/huCD47 cell line.

FIG. 18 is a series of graphs depicting the ability of the IgG₁, IgG₂, and IgG₄PE isotypes of the humanized 108VH4.M4_VL.M1s to promote phagocytosis of human tumor cell line CCRF-CEM by human MDM.

FIG. 19 is a series of graphs depicting the ability of the IgG₁, IgG₂, and IgG₄PE isotypes of the humanized 108VH4.M4_VL.M1 to promote phagocytosis of human tumor cell line Raji by human MDM.

FIG. 20 is a series of graphs depicting the ability of the IgG₁, IgG₂, and IgG₄PE isotypes of the humanized 108VH4.M4_VL.M1 to promote phagocytosis of RBC by human MDM.

FIG. 21 is a graph that shows the in vivo anti-tumor efficacy of the IgG₁, IgG₂, and IgG₄PE isotypes of the humanized 108VH4.M4_VL1.M1, along with the murine B6H12 antibody in a Raji tumor model. In this model, mice were treated with 10 mg/kg antibody doses three times a week.

FIG. 22 is a graph that shows pharmacokinetics of the IgG₁, IgG₂, and IgG₄PE isotypes of the humanized 108VH4.M4_VL.M1 in mouse model. Pharmacokinetics parameters are listed in table 2.

FIG. 23 is a graph that shows the in vivo anti-tumor efficacy of the IgG₄PE isotype of the humanized 108VH4.M4_VL.M1 in a SHP-77 tumor model. In this model, mice were treated with 10 mg/kg antibody doses three times a week.

### DETAILED DESCRIPTION

As used herein, the terms CD47, integrin-associated protein (IAP), ovarian cancer antigen OA3, Rh-related antigen and MER6 are synonymous and may be used interchangeably.

The terms red blood cell(s) (RBC) and erythrocyte(s) are synonymous and used interchangeably herein.

The term agglutination refers to cellular clumping, while the term hemagglutination refers to clumping of a specific subset of cells: red blood cells. Thus, hemagglutination is a type of agglutination.

An antibody refers to a polypeptide (e.g., a tetrameric or single chain polypeptide), comprising the structural and functional characteristics, particularly the antigen binding characteristics, of an immunoglobulin. Typically, a human antibody comprises two identical light chains and two identical heavy chains. Each chain comprises a variable region.

As used herein, the term “antibody” or “antibody molecule” refers to a polypeptide or combination of polypeptides that comprise sufficient sequence from an immunoglobulin heavy chain variable region and/or sufficient sequence from an immunoglobulin light chain variable region, to bind specifically to an antigen. The term comprises full-length antibodies and fragments thereof, e.g., Fab, F(ab′) or F(ab′)₂ fragments. Typically, an antibody molecule comprises heavy chain CDR1, CDR2, and CDR3 and light chain CDR1, CDR2, and CDR3 sequences. Antibody molecules include human, humanized, CDR-grafted antibodies, and antigen binding fragments thereof.

A CDR-grafted antibody is an antibody made by recombinant DNA technology such that a protein or polypeptide comprises the CDRs of an antibody and still binds to the antigen. A CDR-grafted antibody is not identical in structure or amino acid sequence to the antibody from which the CDRs originate.

In certain embodiments, an antibody molecule comprises a protein that comprises at least one immunoglobulin variable region segment, e.g., an amino acid sequence that provides an immunoglobulin variable domain or immunoglobulin variable domain sequence. The term “antibody” includes, for example, a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody. The antibody can also be an engineered protein or antibody-like protein containing at least one immunoglobulin domain (e.g., a fusion protein). The engineered protein or antibody-like protein can also be a bi-specific antibody or a tri-specific antibody, or a dimer, trimer, or multimer antibody, or a diabody, a DVD-Ig, a CODV-Ig, an Affibody®, or a Nanobody®.

As used herein, the term antibody or antibody molecule comprises intact monoclonal antibodies, polyclonal antibodies, single domain antibodies (e.g., shark single domain antibodies (e.g., IgNAR or fragments thereof)), multispecific antibodies (e.g., bi-specific antibodies) formed from at least two intact antibodies, and antibody fragments so long as they exhibit the desired biological activity. Thus, the term “antibody” includes functional variants.

The antibody or antibody molecule can be derived from a mammal, e.g., a rodent, e.g., a mouse or rat, horse, pig, or goat. In certain embodiments, an antibody or antibody molecule is produced using a recombinant cell. In certain embodiments, an antibody or antibody molecule is a chimeric antibody, for example, from mouse, rat, horse, pig, or other species, bearing human constant and/or variable regions domains.

Suitable antibodies (including functional variants) include, but are not limited to, monoclonal, monospecific, polyclonal, polyspecific, human antibodies, primatized antibodies, chimeric antibodies, bi-specific antibodies, humanized antibodies, conjugated antibodies (e.g., antibodies conjugated or fused to other proteins, radiolabels, or cytotoxins), Small Modular ImmunoPharmaceuticals (“SMIPs”), single chain antibodies, cameloid antibodies, and antibody fragments.

In certain embodiments, an antibody molecule is a humanized antibody. A humanized antibody refers to an immunoglobulin comprising a human framework region and one or more CDRs from a non-human, e.g., mouse or rat, immunoglobulin. The immunoglobulin providing the CDRs is often referred to as the “donor” and the human immunoglobulin providing the framework often called the “acceptor,” though in embodiments, no source or no process limitation is implied. Typically, a humanized antibody comprises a humanized light chain and a humanized heavy chain immunoglobulin.

An antibody molecule may comprise a heavy (H) chain variable region (abbreviated herein as V_{H}), and a light (L) chain variable region (abbreviated herein as V_{L}). An antibody may comprise two heavy (H) chain variable regions and two light (L) chain variable regions, or an antibody binding fragment thereof. The light chains of the immunoglobulin may be of types kappa or lambda. The antibody molecule may be glycosylated.

The V_{H} or V_{L} chain of the antibody molecule can further include all or part of a heavy or light chain constant region, to thereby form a heavy or light immunoglobulin chain, respectively. The antibody molecule can be a typical tetramer of two heavy immunoglobulin chains and two light immunoglobulin chains where the two heavy chains are linked by optionally at least one disulfide bond and each pair of heavy and light chains are linked by a disulfide bond. An antibody molecule can also comprise one or both of a heavy (or light) chain immunoglobulin variable region segment. As used herein, the term “heavy (or light) chain immunoglobulin variable region segment,” refers to an entire heavy (or light) chain immunoglobulin variable region, or a fragment thereof, that can bind its antigen. The ability of a heavy or light chain segment to bind antigen is measured with the segment paired with a light or heavy chain, respectively. In certain embodiments, a heavy or light chain segment that is less than a full length variable region may, when paired with the appropriate chain, bind with an affinity that is at least 20, 30, 40, 50, 60, 70, 80, 90, or 95% of what is observed when the full-length chain is paired with a light chain or heavy chain, respectively.

The variable heavy (V_{H}) and variable light (V_{L}) regions can be further subdivided into regions of hypervariability, termed “complementarity determining regions” (CDR), interspersed with regions that are more conserved, termed “framework regions” (FR). Human antibodies have three V_{H}CDRs and three V_{L} CDRs, separated by framework regions FR1-FR4. The extent of the FRs and CDRs has been precisely defined (Kabat, E. A., et al. (1991) SEQUENCES OF PROTEINS OF IMMUNOLOGICAL INTEREST, FIFTH EDITION, U. S. Department of Health and Human Services, NIH Publication No. 91-3242; and Chothia, C. et al. (1987) J. MOL. BIOL. 196:901-917). Each V_{H} and V_{L} is typically composed of three CDRs and four FRs, arranged from amino-terminus to carboxyl-terminus in the following order: FR1, CDR1, FR2, CDR2, FR3, CDR3, and FR4.

The antibody molecule may have a heavy chain constant region chosen from, for example, the heavy chain constant regions of IgG₁, IgG₂, IgG₃, IgG₄, IgM, IgA1, IgA₂, IgD, and IgE; particularly, chosen from, for example, the (e.g., human) heavy chain constant regions of IgG₁, IgG₂, IgG₃, and IgG₄. The antibody molecule may have a light chain constant region chosen from, for example, the (e.g., human) light chain constant regions of kappa or lambda.

The constant region of the antibody may be altered, e.g., mutated, to modify the properties of the antibody (e.g., to increase or decrease one or more of: Fc receptor binding, antibody glycosylation, the number of cysteine residues, effector cell function, and/or complement function). In certain embodiments, the antibody has effector function and can fix complement. In other embodiments, the antibody does not recruit effector cells or fix complement. In other embodiments, the antibody has reduced or no ability to bind an Fc receptor. For example, it is an isotype or subtype, fragment or other mutant, which does not support binding to an Fc receptor, e.g., it has a mutagenized or deleted Fc receptor binding region.

In certain embodiments, the anti-CD47 antibody molecule described herein comprises an IgG₄ constant region. In some further embodiments, the IgG₄ constant region is a wild-type constant region. In other embodiments, the IgG₄ constant region comprises a mutation, e.g., one or both of S228P and L235E, e.g., according to EU numbering (Kabat, E. A., et al., supra). In some embodiments, the anti-CD47 antibody molecule described herein comprises an IgG₁ constant region.

The compositions and methods disclosed herein encompass polypeptides and nucleic acids having the sequences specified, or sequences substantially identical or similar thereto, e.g., sequences at least 85%, 90%, 95% identical or higher to a specified sequence.

In the context of an amino acid sequence, the term “substantially identical” as used herein refers to a first amino acid sequence that contains a sufficient or minimum number of amino acid residues that are: i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., a sequence provided herein.

In the context of nucleotide sequence, the term “substantially identical” as used herein refers to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers to polypeptides that have a substantially identical amino acid sequence to the naturally-occurring sequence, or are encoded by a substantially identical nucleotide sequence, and have one or more activities of the naturally-occurring sequence.

The percent identity between two sequences is a function of the number of identical positions shared by the sequences, taking into account the number of gaps, and the length of each gap, which need to be introduced for optimal alignment of two sequences.

The comparison of sequences and determination of percent identity between two sequences can be accomplished using a mathematical algorithm. In some embodiments, the percent identity between two amino acid sequences is determined using the Needleman and Wunsch ((1970) J. MOL. BIOL. 48:444-453) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet other embodiments, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blosum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS 4:11-17) which has been incorporated into the ALIGN program (version 2.0), using a PAM120 weight residue table, a gap length penalty of 12 and a gap penalty of 4.

It is understood that the molecules disclosed herein may have additional conservative or non-essential amino acid substitutions, which do not have a substantial effect on their functions.

The term “antibody” also includes “antigen-binding fragment” and “antibody fragment,” or the like, e.g., one or more fragments of a full-length antibody that retain the ability to specifically bind to a target antigen of interest. Examples of antigen binding fragments encompassed within the term “antigen-binding fragment” of a full length antibody include (i) a Fab fragment, a monovalent fragment consisting of the V_{L}, V_{H}, C_{L} and CH1 domains; (ii) a F(ab′) or F(ab′)₂ fragment, a bivalent fragment including two Fab fragments linked by a disulfide bridge at the hinge region; (iii) an Fd fragment consisting of the V_{H} and CH1 domains; (iv) an Fv fragment consisting of the V_{L} and V_{H} domains of a single arm of an antibody, (v) an scFv consisting of the V_{L} and V_{H} domains of a single arm of an antibody linked together via a polypeptide linker to produce a single chain Fv (scFv), (vi) a dAb fragment (Ward et al. (1989) NATURE 341:544-546), which consists of a VH domain; and (vii) an isolated complementarity determining region (CDR) that retains functionality.

Antibody fragments or antigen-binding fragments include, e.g., a single chain antibody, a single chain Fv fragment (scFv), an Fd fragment, a Fab fragment, a Fab′ fragment, or an F(ab′)₂ fragment. A scFv fragment is a single polypeptide chain that includes both the heavy and light chain variable regions of the antibody from which the scFv is derived. In addition, intrabodies, minibodies, triabodies, and diabodies are also included in the definition of antibody and are compatible for use in the methods described herein. See, e.g., Todorovska et al. (2001) J Immunol Methods 248(1):47-66; Hudson and Kortt (1999) J Immunol Methods 231(1):177-189; Poljak (1994) Structure 2(12):1121-1123. An antigen-binding fragment can also include the variable region of a heavy chain polypeptide and the variable region of a light chain polypeptide. An antigen-binding fragment can thus comprise the CDRs of the light chain and heavy chain polypeptide of an antibody.

The term “antibody fragment” also can include, e.g., single domain antibodies such as camelized single domain antibodies. See, e.g., Muyldermans et al. (2001) Trends Biochem Sci 26:230-235; PCT application publication nos. WO 94/04678 and WO 94/25591; and U.S. Pat. No. 6,005,079. The term “antibody fragment” also includes single domain antibodies comprising two V_{H} domains with modifications such that single domain antibodies are formed.

The term “immunoglobulin” comprises various broad classes of polypeptides that can be distinguished biochemically. Heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgD, or IgE, respectively. The immunoglobulin subclasses (isotypes), such as IgG1, IgG2, IgG3, IgG4, IgA1, etc., are well characterized and are known to confer functional specialization. Modified versions of each of these classes and isotypes are readily discernable to the skilled artisan in view of the instant disclosure and, accordingly, are within the scope of the instant disclosure. All immunoglobulin classes fall within the scope of the present disclosure. Light chains are classified as either kappa or lambda (κ, λ). Each heavy chain class may be bound with either a kappa or lambda light chain.

An immunoglobulin variable region segment may differ from a reference or consensus sequence. As used herein, to “differ,” means that a residue in the reference sequence or consensus sequence is replaced with either a different residue or an absent or inserted residue.

The heavy and light immunoglobulin chains can be connected by disulfide bonds. The heavy chain constant region typically comprises three constant domains, CH1, CH2 and CH3. The light chain constant region typically comprises a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

An “immunoglobulin domain” refers to a domain from the variable or constant domain of immunoglobulin molecules Immunoglobulin domains typically contain two beta-sheets formed of about seven beta-strands, and a conserved disulfide bond (see, e.g., A. F. Williams and A. N. Barclay (1988) ANN. REV. IMMUNOL. 6:381-405).

As used herein, an “immunoglobulin variable domain sequence” refers to an amino acid sequence that can form the structure of an immunoglobulin variable domain. For example, the sequence may include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that comprises an immunoglobulin variable domain sequence can associate with another immunoglobulin variable domain sequence to form a target binding structure (or “antigen binding site”), e.g., a structure that interacts with the target antigen.

Methods for altering an antibody constant region are known in the art. Antibodies with altered function, e.g. altered affinity for an effector ligand, such as FcR on a cell, or the Cl component of complement can be produced by replacing at least one amino acid residue in the constant portion of the antibody with a different residue (e.g., EP 388,151 A1, U.S. Pat. Nos. 5,624,821 and 5,648,260). Similar types of alterations could be described which if applied to a murine, or other species immunoglobulin would reduce or eliminate these functions.

In some embodiments, a disclosed antibody is a fusion protein. The fusion protein can be constructed recombinantly such that the fusion protein is expressed from a nucleic acid that encodes the fusion protein. The fusion protein can comprise one or more CD47 binding segments and one or more segments that are heterologous to the CD47 binding segment(s). The heterologous sequence can be any suitable sequence, such as, for example, an antigenic tag (e.g., FLAG, polyhistidine, hemagglutinin (“HA”), glutathione-S-transferase (“GST”), or maltose-binding protein (“MBP”)). Heterologous sequences can also be proteins useful as diagnostic or detectable markers, for example, luciferase, green fluorescent protein (“GFP”), or chloramphenicol acetyl transferase (“CAT”). In some embodiments, the heterologous sequence can be a targeting moiety that targets the CD47 binding segment to a cell, tissue, or microenvironment of interest. Methods of constructing such fusion proteins, such as by recombinant DNA technology, are well known in the art.

As used herein, the word “a” or “plurality” before a noun represents one or more of the particular noun. Unless otherwise required by context, singular terms shall include pluralities and plural terms shall include the singular.

As used herein, the terms “subject” and “patient” are used interchangeably. A patient or a subject can be a human patient or a human subject.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting.

All publications, patent applications, patents, sequences, database entries, and other references mentioned herein are incorporated by reference in their entirety. In case of conflict, the present specification, including definitions, will control.

Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the present specification. See e.g., Sambrook et al. (1989) MOLECULAR CLONING: A LABORATORY MANUAL (2nd ed., Cold Spring Harbor Laboratory Press, Cold Spring Harbor, N.Y.). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

CD47

CD47, also known as integrin-associated protein (IAP), ovarian cancer antigen OA3, Rh-related antigen and MER6, is a multi-spanning transmembrane receptor belonging to the immunoglobulin superfamily. CD47 expression and/or activity has been implicated in a number of diseases and disorders, e.g., cancer. CD47 interacts with SIRPα (signal-regulatory-protein α) on macrophages and thereby inhibits phagocytosis. This is a newly discovered mechanism of tumor immune avoidance, and therapeutically targeting CD47 has widespread application in numerous cancers.

The expression of CD47 correlates with worse clinical outcomes in many distinct malignancies, including Acute Lymphocytic Leukemia (ALL), Non-Hodgkin Lymphoma (NHL), Acute Myelogenous Leukemia (AML), glioma, ovarian cancer, glioblastoma, etc. In addition, CD47 has been identified as a cancer stem cell marker in both leukemias and solid tumors (Jaiswal et al., 2009 Cell, 138(2): 271-85; Chan et al., 2010 Curr Opin Urol, 20(5): 393-7; Majeti R et al., 2011 Oncogene, 30(9): 1009-19).

CD47 blocking antibodies have demonstrated anti-tumor activity in multiple mice tumor models. Furthermore, these antibodies have been shown to synergize with other therapeutic antibodies including Herceptin® and Rituxan® in tumor models.

Blocking the interaction of CD47 with SIRPα may enhance phagocytosis of CD47 expressing cells by macrophages (reviewed in Chao et al., 2012 Curr Opin Immunol, 24(2): 225-32). Mice lacking CD47 are markedly resistant to radiation therapy, suggesting a role for targeting CD47 in combination with radiotherapy (Maxhimer et al., 2009 Sci Transl Med, 1(3): 3ra7).

However, prior art existing antibodies to CD47 have been reported to cause hemagglutination of human RBC and anemia. Hemagglutination is an example of a homotypic interaction, such that two CD47 expressing cells are caused to aggregate or clump when treated with a bivalent CD47 binding entity. For example, the CD47 antibody, MABL, as a full IgG or F(ab′)₂, has been reported to cause significant hemagglutination of erythrocytes, and, only when MABL was altered into an scFv or bivalent scFv, was this effect mitigated. (see, e.g., Uno S, Kinoshita Y, Azuma Y et al. Oncol Rep 2007; 17: 1189-94; Kikuchi Y, Uno S, Yoshimura Y et al. Biochem Biophys Res Commun 2004; 315: 912-8). Other known antibodies to CD47, including CC2C6, B6H12 and BRC126, also cause significant hemagglutination of RBCs.

Thus, the aggregation of RBC and anemia represent major limitations of therapeutically targeting CD47 with existing full IgG antibodies and/or SIRPα-Fc fusion proteins.

Anti-CD47 Antibodies of this Disclosure

This disclosure provides anti-CD47 antibodies, including monoclonal antibodies, human antibodies, chimeric antibodies, humanized antibodies, primatized antibodies, bi-specific antibody, conjugated antibodies, a Small Modular ImmunoPharmaceuticals, single chain antibodies, cameloid antibodies, CDR-grafted antibodies, and functional variants of an anti-CD47 antibody (such as, for example, a fusion protein), and fragments and derivatives thereof. These antibodies recognize and bind to CD47 protein, particularly human CD47. These antibodies can modulate, e.g., inhibit, block, antagonize, neutralize or otherwise interfere with CD47 expression, activity and/or signaling; and these antibodies do not cause a significant level of agglutination of cells (also referred to as cell agglutination), including hemagglutination of red blood cells. These antibodies can modulate, e.g., inhibit, block, antagonize, neutralize or otherwise interfere with the interaction between CD47 and SIRPα (signal-regulatory-protein α) (for example, human CD47 and human SIRPα). These antibodies, including fragments, functional variants, and derivatives thereof, may be referred to collectively as “anti-CD47 antibodies of this disclosure,” “disclosed anti-CD47 antibodies,” “disclosed antibodies,” “CD47 antibodies of this disclosure,” and the like.

In some embodiments, the disclosed antibodies include full length IgG antibodies. In some embodiments, the disclosed antibodies include bivalent or multivalent entities.

The disclosed anti-CD47 antibodies are a significant improvement over prior art existing anti-CD47 antibodies that cause hemagglutination of human red blood cells (see, e.g., Kikuchi Y, Uno S, Yoshimura Y et al. Biochem Biophys Res Commun 2004; 315: 912-8). Prior art existing anti-CD47 antibodies include, for example, B6H12, CC2C6, and BRC126 (e.g., U.S. Pat. No. 9,045,541, Uno S, Kinoshita Y, Azuma Y et al. (2007) ONCOL. REP. 17: 1189-94; Kikuchi Y, Uno S, Yoshimura Y et al. (2004) BIOCHEM. BIOPHYS. RES. COMMUN. 315: 912-8). Prior art existing anti-CD47 antibody is one that blocks SIRPα but causes significant hemagglutination of RBCs. The disclosed full-length IgG anti-CD47 antibodies do not agglutinate cells at a significant level.

Anti-CD47 antibody B6H12 is commercially available. It is sold by, for example, ABSCAM (abscam.com/cd47) and Biolegend (Biolegend.com).

In some embodiments, a significant level of agglutination of cells refers to the level of agglutination in the presence of a prior art existing anti-CD47 antibody. In other embodiments, the disclosed anti-CD47 antibodies do not cause a significant level of agglutination when the level of agglutination in the presence of a disclosed anti-CD47 antibody is reduced by at least 5%, at least 10%, at least 20%, at least 30%, at least 40%, at least 50%, at least 60%, at least 70%, at least 80%, at least 90%, or at least 99% compared to the level of agglutination in the presence of a prior art existing anti-CD47 antibody, such as B6H12, CC2C6, and BRC126 (e.g., U.S. Pat. No. 9,045,541, Uno S, Kinoshita Y, Azuma Y et al. (2007) ONCOL. REP. 17: 1189-94; Kikuchi Y, Uno S, Yoshimura Y et al. (2004) BIOCHEM. BIOPHYS. RES. COMMUN. 315: 912-8). In further embodiments, the disclosed anti-CD47 antibodies do not cause a significant level of agglutination of cells at an antibody concentration of between more than about 0 μg/ml and about 100 μg/ml or more than about 0 μg/ml and about 200 μg/ml, such as, for example, at an antibody concentration of 0.3 μg/ml, 0.8 μg/ml, 2.4 μg/ml, 7 μg/ml, 22 μg/ml, 67 μg/ml, and 200 μg/ml.

In some embodiments, a disclosed antibody does not cause significant agglutination of red blood cells and anemia.

Hemagglutination is an example of a homotypic interaction, in which two CD47 expressing cells are caused to aggregate or clump when treated with a bivalent CD47 binding entity. The disclosed anti-CD47 antibodies bind CD47 in a manner that does not promote clumping of CD47 positive cell lines, such as, for example, Raji and CCRF-CEM cells. The lack of significant hemagglutination increases the efficacy of a therapeutic targeting CD47.

In certain embodiments, a disclosed antibody does not (significantly) enhance RBC phagocytosis by macrophage.

The disclosed anti-CD47 antibodies exhibit numerous desirable characteristics, such as, binding human CD47 and cynomolgus monkey (cyno) CD47, potent blocking of the interaction between CD47 and SIRPα, without causing a significant level of hemagglutination, and potent anti-tumor activity in vitro and in vivo.

The disclosed antibodies are also significantly more potent in reducing tumors in tumor models compared to prior art existing anti-CD47 antibodies. In certain embodiments, the ability of macrophages to phagocytose tumor cells in the presence of a disclosed anti-CD47 antibody is increased by at least 5%, at least 10%, at least 20%, at least 30%, at least 40%, at least 50%, at least 60%, at least 70%, at least 80%, at least 90%, or at least 99% compared to the ability of macrophages to phagocytose tumor cells in the presence of a prior art existing anti-CD47 antibody, such as B6H12, CC2C6, and BRC126 (e.g., U.S. Pat. No. 9,045,541, Uno S, Kinoshita Y, Azuma Y et al. (2007) ONCOL. REP. 17:1189-94; Kikuchi Y, Uno S, Yoshimura Y et al. (2004) BIOCHEM. BIOPHYS. RES. COMMUN. 315: 912-8). In some embodiments, a disclosed antibody is a potent anti-tumor agent. In some embodiments, a disclosed antibody reduces tumors. In some embodiments, a disclosed antibody increases the ability of macrophages to phagocytose tumor cells.

In certain embodiments, the disclosed anti-CD47 antibodies block at least 40%, at least 45%, at least 50%, at least 55%, at least 60%, at least 65%, at least 70%, at least 75%, at least 80%, at least 85%, at least 95%, or at least 99% of the interaction between CD47 and SIRPα as compared to the level of interaction between CD47 and SIRPα in the absence of the anti-CD47 antibody described herein.

It is possible to quantitate, without undue experimentation, the level of agglutination, e.g., the level of hemagglutination of RBCs. For example, those skilled in the art will recognize that the level of hemagglutination is ascertained by measuring the area of an RBC dot after performing a hemagglutination assay in the presence of the disclosed anti-CD47 antibodies, as described in the Examples below. In certain embodiments, the area of the RBC dot in the presence of the anti-CD47 antibody disclosed herein is compared to the area of the RBC dot in the absence of an anti-CD47 antibody, i.e., in the presence of zero hemagglutination. In this manner, hemagglutination is quantified relative to a baseline control. A larger RBC dot area corresponds to a higher level of hemagglutination. A large RBC red dot area may appear as a haze. Alternatively, densitometry of the RBC dot may also be utilized to quantitate hemagglutination. The comparison may also be done between a disclosed antibody and a prior art antibody, such as B6H12.

In certain embodiments, less than significant hemagglutination is hemagglutination of human RBCs in the presence of an anti-CD47 antibody disclosed herein is about the same as in the absence of the anti-CD47 antibody. In other embodiments, less than significant hemagglutination is hemagglutination of human RBCs in the presence of an anti-CD47 antibody disclosed herein is about 5%, 10%, 15%, 20%, or 25% in the absence of the anti-CD47 antibody.

In certain embodiments, there is provided an anti-CD47 antibody comprising a heavy chain variable domain (V_{H}) with a heavy chain CDR1 comprising the amino acid sequence of any one of SEQ ID NOs:49, 51, 53, 55, 57, 59, 62-65, 86-87 or a variant thereof comprising up to about 3 (such as about any of 1, 2, or 3) amino acid substitutions; a heavy chain CDR2 comprising the amino acid sequence of any one of SEQ ID NOs:145, 147, 149, 151, 153, 155, 158-161, 182-183 or a variant thereof comprising up to about 3 (such as about any of 1, 2, or 3) amino acid substitutions; and a heavy chain CDR3 comprising the amino acid sequence of any one of SEQ ID NOs:241, 243, 245, 247, 249, 251, 254-257, 278-279 or a variant thereof comprising up to about 3 (such as about any of 1, 2, or 3) amino acid substitutions; and a light chain variable domain (V_{L}) with a light chain CDR1 comprising the amino acid sequence of any one of SEQ ID NOs:50, 52, 54, 56, 58, 60 or a variant thereof comprising up to about 3 (such as about any of 1, 2, or 3) amino acid substitutions; a light chain CDR2 comprising the amino acid sequence of any one of SEQ ID NOs:146, 148, 150, 152, 154, 156, 172-175 or a variant thereof comprising up to about 3 (such as about any of 1, 2, or 3) amino acid substitutions; and a light chain CDR3 comprising the amino acid sequence of any one of SEQ ID NOs:242, 244, 246, 248, 250, 252, 268-271 or a variant thereof comprising up to about 3 (such as about any of 1, 2, or 3) amino acid substitutions. In certain embodiments, there is provided an anti-CD47 antibody comprising a heavy chain variable domain (V_{H}) with a heavy chain CDR1 comprising the amino acid sequence of any one of SEQ ID NOs:49, 51, 53, 55, 57, 59, 62-65, 86-87; a heavy chain CDR2 comprising the amino acid sequence of any one of SEQ ID NOs:145, 147, 149, 151, 153, 155, 158-161, 182-183; and a heavy chain CDR3 comprising the amino acid sequence of any one of SEQ ID NOs:241, 243, 245, 247, 249, 251, 254-257, 278-279; and a light chain variable domain (V_{L}) with a light chain CDR1 comprising the amino acid sequence of any one of SEQ ID NOs:50, 52, 54, 56, 58, 60, 76-79; a light chain CDR2 comprising the amino acid sequence of any one of SEQ ID NOs:146, 148, 150, 152, 154, 156, 172-175; and a light chain CDR3 comprising the amino acid sequence of any one of SEQ ID NOs:242, 244, 246, 248, 250, 252, 268-271.

In certain embodiments, according to any one of the isolated anti-CD47 antibody described above, the said antibody comprise any one of the following:
(1) a V_{H} comprises the heavy chain CDR1, CDR2 and CDR3 sequences having the amino acid sequences of SEQ ID NOs: 49, 145 and 241, respectively, and a V_{L} comprises the light chain CDR1, CDR2 and CDR3 having the amino acid sequences of SEQ ID NOs: 50, 146 and 242, respectively; or a variant thereof comprising up to about 3 (such as about any of 1, 2, or 3) amino acid substitutions in the CDR regions;
(2) a V_{H} comprises the heavy chain CDR1, CDR2 and CDR3 sequences having the amino acid sequences of SEQ ID NOs: 51, 147 and 243, respectively, and a V_{L} comprises the light chain CDR1, CDR2 and CDR3 having the amino acid sequences of SEQ ID NOs: 52, 148 and 244, respectively; or a variant thereof comprising up to about 3 (such as about any of 1, 2, or 3) amino acid substitutions in the CDR regions;
(3) a V_{H} comprises the heavy chain CDR1, CDR2 and CDR3 sequences having the amino acid sequences of SEQ ID NOs: 53, 149 and 245, respectively, and a V_{L} comprises the light chain CDR1, CDR2 and CDR3 having the amino acid sequences of SEQ ID NOs: 54, 150 and 246, respectively; or a variant thereof comprising up to about 3 (such as about any of 1, 2, or 3) amino acid substitutions in the CDR regions;
(4) a V_{H} comprises the heavy chain CDR1, CDR2 and CDR3 sequences having the amino acid sequences of SEQ ID NOs: 55, 151 and 247, respectively, and a V_{L} comprises the light chain CDR1, CDR2 and CDR3 having the amino acid sequences of SEQ ID NOs: 56, 152 and 248, respectively; or a variant thereof comprising up to about 3 (such as about any of 1, 2, or 3) amino acid substitutions in the CDR regions;
(5) a V_{H} comprises the heavy chain CDR1, CDR2 and CDR3 sequences having the amino acid sequences of SEQ ID NOs: 57, 153 and 249, respectively, and a V_{L} comprises the light chain CDR1, CDR2 and CDR3 having the amino acid sequences of SEQ ID NOs: 58, 154 and 250, respectively; or a variant thereof comprising up to about 3 (such as about any of 1, 2, or 3) amino acid substitutions in the CDR regions;
(6) a V_{H} comprises the heavy chain CDR1, CDR2 and CDR3 sequences having the amino acid sequences of SEQ ID NOs: 59, 155 and 251, respectively, and a V_{L} comprises the light chain CDR1, CDR2 and CDR3 having the amino acid sequences of SEQ ID NOs: 60, 156 and 252, respectively; or a variant thereof comprising up to about 3 (such as about any of 1, 2, or 3) amino acid substitutions in the CDR regions;
(7) a V_{H} comprises the heavy chain CDR1, CDR2 and CDR3 sequences having the amino acid sequences of SEQ ID NOs:62, 158 and 254, respectively, and a V_{L} comprises the light chain CDR1, CDR2 and CDR3 having the amino acid sequences of SEQ ID NOs: 76, 172 and 268, respectively;
(8) a V_{H} comprises the heavy chain CDR1, CDR2 and CDR3 sequences having the amino acid sequences of SEQ ID NOs:63, 159 and 255, respectively, and a V_{L} comprises the light chain CDR1, CDR2 and CDR3 having the amino acid sequences of SEQ ID NOs: 77, 173 and 269, respectively; or a variant thereof comprising up to about 3 (such as about any of 1, 2, or 3) amino acid substitutions in the CDR regions;
(9) a V_{H} comprises the heavy chain CDR1, CDR2 and CDR3 sequences having the amino acid sequences of SEQ ID NOs:64, 160 and 256, respectively, and a V_{L} comprises the light chain CDR1, CDR2 and CDR3 having the amino acid sequences of SEQ ID NOs: 78, 174 and 270, respectively; or a variant thereof comprising up to about 3 (such as about any of 1, 2, or 3) amino acid substitutions in the CDR regions;
(10) a V_{H} comprises the heavy chain CDR1, CDR2 and CDR3 sequences having the amino acid sequences of SEQ ID NOs:65, 161 and 257, respectively, and a V_{L} comprises the light chain CDR1, CDR2 and CDR3 having the amino acid sequences of SEQ ID NOs: 79, 175 and 271, respectively; or a variant thereof comprising up to about 3 (such as about any of 1, 2, or 3) amino acid substitutions in the CDR regions;
(11) a V_{H} comprises the heavy chain CDR1, CDR2 and CDR3 sequences having the amino acid sequences of SEQ ID NOs:65, 161 and 257, respectively, and a V_{L} comprises the light chain CDR1, CDR2 and CDR3 having the amino acid sequences of SEQ ID NOs: 76, 172 and 268, respectively; or a variant thereof comprising up to about 3 (such as about any of 1, 2, or 3) amino acid substitutions in the CDR regions;
(12) a V_{H} comprises the heavy chain CDR1, CDR2 and CDR3 sequences having the amino acid sequences of SEQ ID NOs:86, 182 and 278, respectively, and a V_{L} comprises the light chain CDR1, CDR2 and CDR3 having the amino acid sequences of SEQ ID NOs: 56, 152 and 248, respectively; or a variant thereof comprising up to about 3 (such as about any of 1, 2, or 3) amino acid substitutions in the CDR regions; and
(13) a V_{H} comprises the heavy chain CDR1, CDR2 and CDR3 sequences having the amino acid sequences of SEQ ID NOs:87, 183 and 279, respectively, and a V_{L} comprises the light chain CDR1, CDR2 and CDR3 having the amino acid sequences of SEQ ID NOs: 56, 152 and 248, respectively; or a variant thereof comprising up to about 3 (such as about any of 1, 2, or 3) amino acid substitutions in the CDR regions.

In certain embodiments, according to any one of the isolated anti-CD47 antibody described above, the said antibody comprise any one of the following:
(1) a V_{H} comprises the heavy chain CDR1, CDR2 and CDR3 sequences having the amino acid sequences of SEQ ID NOs: 49, 145 and 241, respectively, and a V_{L} comprises the light chain CDR1, CDR2 and CDR3 having the amino acid sequences of SEQ ID NOs: 50, 146 and 242, respectively;
(2) a V_{H} comprises the heavy chain CDR1, CDR2 and CDR3 sequences having the amino acid sequences of SEQ ID NOs: 51, 147 and 243, respectively, and a V_{L} comprises the light chain CDR1, CDR2 and CDR3 having the amino acid sequences of SEQ ID NOs: 52, 148 and 244, respectively;
(3) a V_{H} comprises the heavy chain CDR1, CDR2 and CDR3 sequences having the amino acid sequences of SEQ ID NOs: 53, 149 and 245, respectively, and a V_{L} comprises the light chain CDR1, CDR2 and CDR3 having the amino acid sequences of SEQ ID NOs: 54, 150 and 246, respectively;
(4) a V_{H} comprises the heavy chain CDR1, CDR2 and CDR3 sequences having the amino acid sequences of SEQ ID NOs: 55, 151 and 247, respectively, and a V_{L} comprises the light chain CDR1, CDR2 and CDR3 having the amino acid sequences of SEQ ID NOs: 56, 152 and 248, respectively;
(5) a V_{H} comprises the heavy chain CDR1, CDR2 and CDR3 sequences having the amino acid sequences of SEQ ID NOs: 57, 153 and 249, respectively, and a V_{L} comprises the light chain CDR1, CDR2 and CDR3 having the amino acid sequences of SEQ ID NOs: 58, 154 and 250, respectively;
(6) a V_{H} comprises the heavy chain CDR1, CDR2 and CDR3 sequences having the amino acid sequences of SEQ ID NOs: 59, 155 and 251, respectively, and a V_{L} comprises the light chain CDR1, CDR2 and CDR3 having the amino acid sequences of SEQ ID NOs: 60, 156 and 252, respectively;
(7) a V_{H} comprises the heavy chain CDR1, CDR2 and CDR3 sequences having the amino acid sequences of SEQ ID NOs:62, 158 and 254, respectively, and a V_{L} comprises the light chain CDR1, CDR2 and CDR3 having the amino acid sequences of SEQ ID NOs: 76, 172 and 268, respectively;
(8) a V_{H} comprises the heavy chain CDR1, CDR2 and CDR3 sequences having the amino acid sequences of SEQ ID NOs:63, 159 and 255, respectively, and a V_{L} comprises the light chain CDR1, CDR2 and CDR3 having the amino acid sequences of SEQ ID NOs: 77, 173 and 269, respectively;
(9) a V_{H} comprises the heavy chain CDR1, CDR2 and CDR3 sequences having the amino acid sequences of SEQ ID NOs:64, 160 and 256, respectively, and a V_{L} comprises the light chain CDR1, CDR2 and CDR3 having the amino acid sequences of SEQ ID NOs: 78, 174 and 270, respectively;
(10) a V_{H} comprises the heavy chain CDR1, CDR2 and CDR3 sequences having the amino acid sequences of SEQ ID NOs:65, 161 and 257, respectively, and a V_{L} comprises the light chain CDR1, CDR2 and CDR3 having the amino acid sequences of SEQ ID NOs: 79, 175 and 271, respectively;
(11) a V_{H} comprises the heavy chain CDR1, CDR2 and CDR3 sequences having the amino acid sequences of SEQ ID NOs:65, 161 and 257, respectively, and a V_{L} comprises the light chain CDR1, CDR2 and CDR3 having the amino acid sequences of SEQ ID NOs: 76, 172 and 268, respectively;
(12) a V_{H} comprises the heavy chain CDR1, CDR2 and CDR3 sequences having the amino acid sequences of SEQ ID NOs:86, 182 and 278, respectively, and a V_{L} comprises the light chain CDR1, CDR2 and CDR3 having the amino acid sequences of SEQ ID NOs: 56, 152 and 248, respectively; and
(13) a V_{H} comprises the heavy chain CDR1, CDR2 and CDR3 sequences having the amino acid sequences of SEQ ID NOs:87, 183 and 279, respectively, and a V_{L} comprises the light chain CDR1, CDR2 and CDR3 having the amino acid sequences of SEQ ID NOs: 56, 152 and 248, respectively.

In certain embodiments, this disclosure provides anti-CD47 antibodies comprising a variable heavy chain selected from SEQ ID NOs: 349, 351, 353, 355, 357, 359, 361-373, 380-383, 388-392 and a variable light chain selected from SEQ ID NOs:350, 352, 354, 356, 358, 360, 374-379, 384-387 and 393-396. In certain embodiments, the anti-CD47 antibodies comprise a variable heavy chain that is at least 90%, 91%, 92%, 93%, 94%, 95%, 96%, 97%, 98%, 99% or more identical to the sequence set forth in at least one of SEQ ID NOs: 349, 351, 353, 355, 357, 359, 361-373, 380-383, 388-392 and a variable light chain that is at least 90%, 91%, 92%, 93%, 94%, 95%, 96%, 97%, 98%, 99% or more identical to the sequence set forth in at least the sequence set forth in at least one of SEQ ID NOs: from 350, 352, 354, 356, 358, 360, 374-379, 384-387 and 393-396.

The anti-CD47 antibodies provided herein exhibit inhibitory activity, for example, by inhibiting CD47 expression (e.g., inhibiting cell surface expression of CD47), activity, and/or signaling, or by interfering with the interaction between CD47 and SIRPα. The antibodies provided herein completely or partially reduce or otherwise modulate CD47 expression or activity upon binding to, or otherwise interacting with, CD47, e.g., a human CD47. The reduction or modulation of a biological function of CD47 is complete, significant, or partial upon interaction between the antibodies and the human CD47 polypeptide and/or peptide.

The antibodies are considered to completely inhibit CD47 expression or activity when the level of CD47 expression or activity in the presence of the antibody is decreased by at least 95%, e.g., by 96%, 97%, 98%, 99% or 100% as compared to the level of CD47 expression or activity in the absence of interaction, e.g., binding, with the antibody described herein.

The anti-CD47 antibodies are considered to significantly inhibit CD47 expression or activity when the level of CD47 expression or activity in the presence of the CD47 antibody is decreased by at least 50%, e.g., 55%, 60%, 75%, 80%, 85% or 90% as compared to the level of CD47 expression or activity in the absence of binding with a anti-CD47 antibody described herein. The antibodies are considered to partially inhibit CD47 expression or activity when the level of CD47 expression or activity in the presence of the antibody is decreased by less than 95%, e.g., 10%, 20%, 25%, 30%, 40%, 50%, 60%, 75%, 80%, 85% or 90% as compared to the level of CD47 expression or activity in the absence of interaction, e.g., binding, with an antibody described herein.

The amount of antibody sufficient to treat or prevent cancer in the subject is, for example, an amount that is sufficient to reduce CD47 signaling. For example, the amount of antibody sufficient to treat or prevent cancer in the subject is an amount that is sufficient to reduce the phagocytic inhibitory signal in macrophages generated by CD47/SIRPα interaction in the CD47/SIRPα signaling axis, i.e., a disclosed antibody promotes macrophage-mediated phagocytosis of a CD47-expressing cell.

As used herein, the term “reduced,” in relation to the phagocytic inhibitory signal in macrophages generated by CD47/SIRPα interaction in the CD47/SIRPα signaling axis, refers to a decreased CD47 signaling in the presence of a disclosed anti-CD47 antibody. CD47 mediated signaling is decreased when the level of CD47 signaling in the presence of a disclosed anti-CD47 antibody is greater than or equal to 5%, 10%, 20%, 25%, 30%, 40%, 50%, 60%, 70%, 75%, 80%, 90%, 95%, 99%, or 100% lower than a control level of CD47 signaling (i.e., the level of CD47 signaling in the absence of the antibody). Level of CD47 signaling is measured using any of a variety of standard techniques, such as, by way of non-limiting example, measurement of down-stream gene activation, and/or luciferase reporter assays responsive to CD47 activation. Those skilled in the art will appreciate that the level of CD47 signaling can be measured using a variety of assays, including, for example, commercially available kits.

In some embodiments, the disclosed anti-CD47 antibody or immunologically active fragment thereof is an IgG isotype. In some embodiments, the constant region of the antibody is of human IgG₁ isotype, having an amino acid sequence:

(SEQ ID NO: 408)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, the human IgG₁ constant region is modified at amino acid Asn297 (Boxed, Kabat Numbering) to prevent to glycosylation of the antibody, for example Asn297Ala (N297A). In some embodiments, the constant region of the antibody is modified at amino acid Leu235 (Kabat Numbering) to alter Fc receptor interactions, for example Leu235Glu (L235E) or Leu235Ala (L235A). In some embodiments, the constant region of the antibody is modified at amino acid Leu234 (Kabat Numbering) to alter Fc receptor interactions, e.g., Leu234Ala (L234A). In some embodiments, the constant region of the antibody is altered at both amino acid 234 and 235, for example Leu234Ala and Leu235Ala (L234A/L235A) (EU index of Kabat et al 1991 Sequences of Proteins of Immunological Interest).

In some embodiments, the constant region of the antibody is of human IgG₂ isotype, having an amino acid sequence:

(SEQ ID NO: 409)
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK

In some embodiments, the human IgG₃ constant region is modified at amino acid Asn297 (Boxed, Kabat Numbering) to prevent to glycosylation of the antibody, e.g., Asn297Ala (N297A). In some embodiments, the human IgG₃ constant region is modified at amino acid 435 to extend the half-life, e.g., Arg435His (R435H) (EU index of Kabat et al 1991 Sequences of Proteins of Immunological Interest).

In some embodiments, the constant region of the antibody is of human IgG₄ isotype, having an amino acid sequence:

(SEQ ID NO: 410)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK

In some embodiments, the human IgG₄ constant region is modified within the hinge region to prevent or reduce strand exchange, e.g., Ser228Pro (S228P). In other embodiments, the human IgG₄ constant region is modified at amino acid 235 to alter Fc receptor interactions, e.g., Leu235Glu (L235E). In some embodiments, the human IgG₄ constant region is modified within the hinge and at amino acid 235, e.g., Ser228Pro and Leu235Glu (S228P/L235E), having an amino acid sequence:

(SEQ ID NO: 411)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFEGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK

In some embodiments, the human IgG constant region is modified to enhance FcRn binding. Examples of Fc mutations that enhance binding to FcRn are Met252Tyr, Ser254Thr, Thr256Glu (M252Y, S254T, T256E, respectively) (Kabat numbering, Dall'Acqua et al. 2006 I. Biol Chem Vol 281(33) 23514-23524), or Met428Leu and Asn434Ser (M428L, N434S) (Zalevsky et al. 2010 Nature Biotech, Vol 28(2) 157-159). (EU index of Kabat et al. 1991 Sequences of Proteins of Immunological Interest). In some embodiments, the human IgG constant region is modified to alter antibody-dependent cellular cytotoxicity (ADCC) and/or complement-dependent cytotoxicity (CDC), e.g., the amino acid modifications described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al., 1992 J Immunol, 148: 3461-3468; Reviewed in Kaneko and Niwa, 2011 Biodrugs, 25(1): 1-11.

In some embodiments, the human IgG constant region is modified to induce heterodimerization. For example, having an amino acid modification within the CH3 domain at Thr366, which when replaced with a bulkier amino acid, e.g., Try (T366W), can preferentially pair with a second CH3 domain having amino acid modifications to less bulky amino acids at positions Thr366, Leu368, and Tyr407, e.g., Ser, Ala and Val, respectively (T366S/L368A/Y407V). Heterodimerization via CH3 modifications can be further stabilized by the introduction of a disulfide bond, for example by changing Ser354 to Cys (S354C) and Y349 to Cys (Y349C) on opposite CH3 domains (Reviewed in Carter, 2001 Journal of Immunological Methods, 248: 7-15).

In some embodiments, a disclosed anti-CD47 antibody comprises a variable heavy (V_{H}) chain region selected from the group consisting of SEQ ID NOs: 349, 351, 353, 355, 357, 359, 361-373, 380-383, 388-392. The disclosed anti-CD47 antibody optionally comprises a variable light (V_{L}) chain region selected from the group consisting of sequences from SEQ ID NOs: 350, 352, 354, 356, 358, 360, 374-379, 384-387 and 393-396. In some embodiments, the disclosed anti-CD47 antibody comprises a V_{H} chain region selected from the group consisting of sequences from SEQ ID NOs: 349, 351, 353, 355, 357, 359, 361-373, 380-383, 388-392 and a V_{L} chain region selected from the group consisting of sequences from SEQ ID NOs: 350, 352, 354, 356, 358, 360, 374-379, 384-387 and 393-396. The disclosed antibodies also include antibodies having a variable heavy chain that is at least 90%, 91%, 92%, 93%, 94%, 95%, 96%, 97%, 98%, 99% or more identical to a sequence set forth in at least one of SEQ ID NOs: 349, 351, 353, 355, 357, 359, 361-373, 380-383, 388-392 and a variable light chain that is at least 90%, 91%, 92%, 93%, 94%, 95%, 96%, 97%, 98%, 99% or more identical to a sequence set forth in at least one of SEQ ID NOs: 350, 352, 354, 356, 358, 360, 374-379, 384-387 and 393-396.

In other embodiments, the disclosed anti-CD47 antibody comprises a V_{H} region provided in any one of SEQ ID NOs: 349, 351, 353, 355, 357, 359, 361-373, 380-383, 388-392 paired with a V_{L} region provided in any one of SEQ ID NOs: 350, 352, 354, 356, 358, 360, 374-379, 384-387 and 393-396.

In other embodiments, the disclosed anti-CD47 antibody comprises a V_{H} region provided in any one of SEQ ID NOs: 349, 351, 353, 355, 357, 359, 361-373, 380-383, 388-392 paired with a V_{L} region provided in any one of SEQ ID NOs: 350, 352, 354, 356, 358, 360, 374-379, 384-387 and 393-396.

In certain embodiments, the disclosed anti-CD47 antibodies bind to CD47 in a head to side orientation that positions the heavy chain near the membrane of CD47 expressing cell, while the light chain occludes the SIRPα binding site on CD47. In other embodiments, the disclosed anti-CD47 antibodies bind to CD47 in a head to side orientation that positions the light chain near the membrane of CD47 expressing cell, while the heavy chain occludes the SIRPα binding site on CD47.

Also provided is an isolated antibody or an immunologically active fragment thereof which competes with the CD47 antibodies described herein for preventing CD47 from interacting with SIRPα.

Monoclonal antibodies described herein have the ability to bind CD47, to inhibit the binding of SIRPα to CD47, decrease CD47-SIRPα-mediated signaling, promote phagocytosis of tumor cells, and to inhibit tumor growth and/or migration. Inhibition is determined, for example, using the cellular assay described herein in the Examples.

Exemplary antibodies described herein include the murine CD47 antibodies, the chimeric version of 98E2E12, 107F11F10 and 108C10A6, and humanized variants of 108C10A6.

Exemplary monoclonal antibodies of this disclosure include, for example, murine antibodies having a variable heavy chain region (V_{H}) and/or variable light (V_{L}) chain region shown in the sequences below.

55F2C4-VH
SEQ ID NO: 349
QVQLQQSGPQLVRPGASVKISCKASGYSFTNYWMHWMKQRPGQGLEWIGM
IDPSDSETRLNQQFKDKATLAVDKSSSTAYMQLSSPTSEDSAVYYCARLG
RYYFDYWGQGTTLTVSS
55F2C4-VL
SEQ ID NO: 350
NIVMTQSPKSMYVSVGERVTLICRASEIVGTYVSWYQQKPEQSPKLLIYG
ASNRYTGVPDRFTGSRSATDFSLTISNVQAEDLADYLCGQSYDSPYTFGG
GTKLEIK
98E2E7/98E2E12-SEQ ID NO: 351VH
QVQLQQSGPQLVRPGASVKISCKASGYSFTNHWMHWMKQRPGQGLEWIGM
IDPSDSETRLNQQFKDKATLTVDKSSSTAYMQLSSPTSEDSAVFYCARLG
RYYFDYWGQGTTLTVSS
98E2E7/98E2E12-VL
SEQ ID NO: 352
NIVMTQSPKSMSVSVGERVTLSCRASDIVGTYVSWYQQKPEQSPKLLIYG
ASNRYTGVPDRFTGSRSATDFSLTISNVQAEDLADYLCGQSYDSPYTFGG
GTKLEIK
98G5F6/98G5F11-VH
SEQ ID NO: 353
QVQLQQSGPQLVRPGASVKISCKASGYSFTNYWMHWMKQRPGQGLEWIGM
IDPSDSETRLNQQFKDKATLTVDKSSSTAYMQLSSPTSEDSAVYYCARLG
RYYFDFWGQGTTLTVSS
98G5F6/98G5F11-VL
SEQ ID NO: 354
NIVMTQSPKSMSVSVGERVTLSCRASEIVGTYVSWYQQKPEQSPKLLIYG
ASNRFTGVPDRFTGSRSATDFSLTISNVQAEDLADYLCGQSYDSPYTFGG
GTKLEIK
107F11B11/107F11C4/107F11F10-VH
SEQ ID NO: 355
EVQLQQSGAEFVKPGASVKLSCTASGFNIEDTYMHWVKQRPEQGLEWIGM
IDPANGKTKYGPRFQDKATVTADTSSNTANLQLSSLTSEDTAVYYCADGI
GYYVGAMDYWGQGTSVTVSS
107F11B11/107F11C4/107F11F10-VL
SEQ ID NO: 356
DIQMNQSPSSLSASLGDTITITCHASQNINVWLSWYQQKPGNIPKLLIYK
ASNLHTGVPSRFSGSGSGTGFTLTISSLQPEDIATYYCQQGHSYPYTFGG
GTKLEIK
108C10A6/108C10F5-VH
SEQ ID NO: 357
QMQLQQSGPQLVRPGASVKISCKTSGYSFTHHWIHWMKQRPGQGLEWIGM
IDPSDSETRLSQKFKDKATLTVDASSSTAYMQLNSPTSEDSALYFCARLG
RYYFDYWGQGTTLTVSS
108C10A6/108C10F5-VL
SEQ ID NO: 358
NIVMTQSPRSMSMSVGERVTLSCKASENVGTYISWYQQKPDQSPKLLIYG
ASNRYTGVPDRFTGSGSGTDFTLTISTVQAEDLADYHCGESYGHLYTFGG
GTKLEIK112E5D9/112E5F2/112E5H7-VH
SEQ ID NO: 359
QVQLQQSGPQLVRPGASVKISCKASGYSFTNNWMHWMKQRPGQGLEWIGM
IDPSDSETRLNQQFRDKATLTVDKTSSTAYMQLSSPTSEDSAVYYCARLG
RYYFDYWGLGTTLTVSS
112E5D9/112E5F2/112E5H7-VL
SEQ ID NO: 360
NIVMTQSPKSMSVSVGERVTMNCRASEIVGTYVSWYQQKPEQSPKLLIYG
AFNRYTGVPDRFTGSRSGTDFSLNISNVQAEDLADYLCGQSYDSPYTFGG
GTKLEIK

Exemplary disclosed anti-CD47 monoclonal antibodies include, for example, chimeric antibodies having a variable heavy chain region (V_{H}) and/or variable light (V_{L}) chain region shown in the sequences below.

108C10A6_VH-huIgG1CH
SEQ ID NO: 397
QMQLQQSGPQLVRPGASVKISCKTSGYSFTHHWIHWMKQRPGQGLEWIGM
IDPSDSETRLSQKFKDKATLTVDASSSTAYMQLNSPTSEDSALYFCARLG
RYYFDYWGQGTTLTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
108C10A6_VL-hIgKCL
SEQ ID NO: 398
NIVMTQSPRSMSMSVGERVTLSCKASENVGTYISWYQQKPDQSPKLLIYG
ASNRYTGVPDRFTGSGSGTDFTLTISTVQAEDLADYHCGESYGHLYTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
98E2E12_VH-huIgG1CH
SEQ ID NO: 404
QVQLQQSGPQLVRPGASVKISCKASGYSFTNHWMHWMKQRPGQGLEWIGM
IDPSDSETRLNQQFKDKATLTVDKSSSTAYMQLSSPTSEDSAVFYCARLG
RYYFDYWGQGTTLTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
98E2E12_VL-hIgKCL
SEQ ID NO: 405
NIVMTQSPKSMSVSVGERVTLSCRASDIVGTYVSWYQQKPEQSPKLLIYG
ASNRYTGVPDRFTGSRSATDFSLTISNVQAEDLADYLCGQSYDSPYTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
107F11F10-VH-huIgG1CH
SEQ ID NO: 406
EVQLQQSGAEFVKPGASVKLSCTASGFNIEDTYMHWVKQRPEQGLEWIGM
IDPANGKTKYGPRFQDKATVTADTSSNTANLQLSSLTSEDTAVYYCADGI
GYYVGAMDYWGQGTSVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
107F11F10-VL-hIgKCL
SEQ ID NO: 407
DIQMNQSPSSLSASLGDTITITCHASQNINVWLSWYQQKPGNIPKLLIYK
ASNLHTGVPSRFSGSGSGTGFTLTISSLQPEDIATYYCQQGHSYPYTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Exemplary disclosed anti-CD47 antibodies include, for example, humanized antibodies having a variable heavy chain region (V_{H}) and/or variable light (V_{L}) chain region shown in the sequences below.

108VH1
SEQ ID NO: 366
QVQLVQSGAEVKKPGSSVKVSCKASGYSFTHHWIHWVRQAPGQGLEWMGM
IDPSDSETRLSQKFKDRVTITADKSTSTAYMELSSLRSEDTAVYYCARLG
RYYFDYWGQGTTVTVSS
108VH2
SEQ ID NO: 367
EVQLVQSGAEVKKPGSSVKVSCKASGYSFTHHWIHWMRQAPGQGLEWIGM
IDPSDSETRLSQKFKDRVTITADKSTSTAYMELSSLRSEDTAVYFCARLG
RYYFDYWGQGTTVTVSS
108VH3
SEQ ID NO: 368
EVQLVQSGAEVKKPGSSVKVSCKASGYSFTHHWIHWMRQAPGQGLEWIGM
IDPSDSETRLSQKFKDRATLTVDKSTSTAYMELSSLRSEDTAVYFCARLG
RYYFDYWGQGTTVTVSS
108Vha
SEQ ID NO: 369
EVQLVQSGAEVKKPGSSVKVSCKTSGYSFTHHWIHWMKQAPGQGLEWIGM
IDPSDSETRLSQKFKDKATLTVDKSTSTAYMELSSLRSEDTAVYFCARLG
RYYFDYWGQGTTVTVSS
108VH4.M4
SEQ ID NO: 373
EVQLVQSGAEVKKPGSSVKVSCKASGYSFTHHWIHWVRQAPGQGLEWMGM
IDASDSETRLSQKFKDRVTITADKSTSTAYMELSSLRSEDTAVYYCARLG
RYYFDYWGQGTTVTVSS
108VL1.M1
SEQ ID NO: 377
EIVLTQSPATLSLSPGERATLSCRASENVGTYISWYQQKPGQAPRLLIYG
ASNRYTGIPARFSGSGSGTDFTLTISSLEPEDFAVYYCGESYGHLYTFGG
GTKVEIK
108VL2.M1
SEQ ID NO: 378
EIVLTQSPATLSLSPGERATLSCRASENVGTYISWYQQKPGQAPRLLIYG
ASNRYTGVPARFSGSGSGTDFTLTISSLEPEDFAVYHCGESYGHLYTFGG
GTKVEIK
108VL3.M1
SEQ ID NO: 379
EIVLTQSPATLSLSPGERVTLSCRASENVGTYISWYQQKPGQAPRLLIYG
ASNRYTGVPARFSGSGSGTDFTLTISSVEPEDFAVYHCGESYGHLYTFGG
GTKLEIK
108VH4.M4-huIgG1
SEQ ID NO: 399
EVQLVQSGAEVKKPGSSVKVSCKASGYSFTHHWIHWVRQAPGQGLEWMGM
IDASDSETRLSQKFKDRVTITADKSTSTAYMELSSLRSEDTAVYYCARLG
RYYFDYWGQGTTVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
108VH4.M4-hIgG2
SEQ ID NO: 400
EVQLVQSGAEVKKPGSSVKVSCKASGYSFTHHWIHWVRQAPGQGLEWMGM
IDASDSETRLSQKFKDRVTITADKSTSTAYMELSSLRSEDTAVYYCARLG
RYYFDYWGQGTTVTVSSASTKGPSVFPLAPCSRSTSESTAALGCLVKDYF
PEPVTVSWNSGALTSGVHTFPAVLQSSGLYSLSSVVTVPSSNFGTQTYTC
NVDHKPSNTKVDKTVERKCCVECPPCPAPPVAGPSVFLFPPKPKDTLMIS
RTPEVTCVVVDVSHEDPEVQFNWYVDGVEVHNAKTKPREEQFNSTFRVVS
VLTVVHQDWLNGKEYKCKVSNKGLPAPIEKTISKTKGQPREPQVYTLPPS
REEMTKNQVSLTCLVKGFYPSDIAVEWESNGQPENNYKTTPPMLDSDGSF
FLYSKLTVDKSRWQQGNVFSCSVMHEALHNHYTQKSLSLSPGK
108VH4.M4-hIgG4PE
SEQ ID NO: 401
EVQLVQSGAEVKKPGSSVKVSCKASGYSFTHHWIHWVRQAPGQGLEWMGM
IDASDSETRLSQKFKDRVTITADKSTSTAYMELSSLRSEDTAVYYCARLG
RYYFDYWGQGTTVTVSSASTKGPSVFPLAPCSRSTSESTAALGCLVKDYF
PEPVTVSWNSGALTSGVHTFPAVLQSSGLYSLSSVVTVPSSSLGTKTYTC
NVDHKPSNTKVDKRVESKYGPPCPPCPAPEFEGGPSVFLFPPKPKDTLMI
SRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK
108VH4.M4-hIgG4P
SEQ ID NO: 403
EVQLVQSGAEVKKPGSSVKVSCKASGYSFTHHWIHWVRQAPGQGLEWMGM
IDASDSETRLSQKFKDRVTITADKSTSTAYMELSSLRSEDTAVYYCARLG
RYYFDYWGQGTTVTVSSASTKGPSVFPLAPCSRSTSESTAALGCLVKDYF
PEPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLMI
SRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK
108VL1.M1-hIgKCL
SEQ ID NO: 402
EIVLTQSPATLSLSPGERATLSCRASENVGTYISWYQQKPGQAPRLLIYG
ASNRYTGIPARFSGSGSGTDFTLTISSLEPEDFAVYYCGESYGHLYTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
107VH1
SEQ ID NO: 381
EVQLVQSGAEVKKPGSSVKVSCKASGFNIEDTYMHWVRQAPGQGLEWMGM
IDPANGKTKYGPRFQDRVTITADKSTSTAYMELSSLRSEDTAVYYCARGI
GYYVGAMDYWGQGTTVTVSS
107VH2
SEQ ID NO: 382
EVQLVQSGAEVKKPGSSVKVSCKASGFNIEDTYMHWVRQAPGQGLEWIGM
IDPANGKTKYGPRFQDRVTITADKSTSTAYMELSSLRSEDTAVYYCADGI
GYYVGAMDYWGQGTTVTVSS
107VH3
SEQ ID NO: 383
EVQLVQSGAEVKKPGSSVKVSCKASGFNIEDTYMHWVRQAPGQGLEWIGM
IDPANGKTKYGPRFQDRATVTADKSTSTAYMELSSLRSEDTAVYYCADGI
GYYVGAMDYWGQGTTVTVSS
107VL1
SEQ ID NO: 385
DIQMTQSPSSLSASVGDRVTITCHASQNINVWLSWYQQKPGKAPKLLIYK
ASNLHTGVPSRFSGSGSGTDFTLTISSLQPEDFATYYCQQGHSYPYTFGG
GTKVEIK
107VL1-M1
SEQ ID NO: 386
DIQMTQSPSSLSASVGDRVTITCRASQNINVWLSWYQQKPGKAPKLLIYK
ASNLHTGVPSRFSGSGSGTDFTLTISSLQPEDFATYYCQQGHSYPYTFGG
GTKVEIK
107VL2.M1
SEQ ID NO: 387
DIQMTQSPSSLSASVGDRITITCRASQNINVWLSWYQQKPGKAPKLLIYK
ASNLHTGVPSRFSGSGSGTDFTLTISSLQPEDFATYYCQQGHSYPYTFGG
GTKLEIK

Any suitable procedure known in the art may be used for making monoclonal antibodies directed against CD47, or against derivatives, fragments, analogs homologs or orthologs thereof. (See, e.g., Antibodies: A Laboratory Manual, Harlow E, and Lane D, 1988, Cold Spring Harbor Laboratory Press, Cold Spring Harbor, N.Y.). Fully human antibodies are antibody molecules in which the entire sequence of both the light chain and the heavy chain, including the CDRs, arise from human genes. Such antibodies are termed “human antibodies” or “fully human antibodies” herein. Human monoclonal antibodies are prepared, for example, using the procedures described in the Examples provided below. Human monoclonal antibodies can be also prepared by using the trioma technique; the human B-cell hybridoma technique (Kozbor, et al., 1983 Immunol Today 4: 72); and the EBV hybridoma technique to produce human monoclonal antibodies (Cole, et al., 1985 In: MONOCLONAL ANTIBODIES AND CANCER THERAPY, Alan R. Liss, Inc., pp. 77-96). Human monoclonal antibodies may be utilized and may be produced by using human hybridomas (Cote, et al., 1983. Proc Natl Acad Sci USA 80: 2026-2030) or by transforming human B-cells with Epstein Barr Virus in vitro (Cole, et al., 1985 In: MONOCLONAL ANTIBODIES AND CANCER THERAPY, Alan R. Liss, Inc., pp. 77-96).

DNA sequence of human CD47 is known in the art. A DNA sequence of human CD47 is provided as SEQ ID NO: 412.

Antibodies may be purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal antibodies that modulate, block, inhibit, reduce, antagonize, neutralize or otherwise interfere with CD47- and/or CD47/SIRPα-mediated cell signaling are generated, e.g., by immunizing an animal with membrane bound and/or soluble CD47, such as, human CD47 or an immunogenic fragment, derivative or variant thereof.

Alternatively, the animal is immunized with cells transfected with a vector containing a nucleic acid molecule encoding CD47 such that CD47 is expressed and associated with the surface of the transfected cells. Alternatively, the antibodies are obtained by screening a library that contains antibody or antigen binding domain sequences for binding to CD47. This library is prepared, e.g. in bacteriophage as protein or peptide fusions to a bacteriophage coat protein that is expressed on the surface of assembled phage particles and the encoding DNA sequences contained within the phage particles (i.e. “phage displayed library”). Hybridomas resulting from myeloma/B cell fusions are then screened for reactivity to CD47.

Monoclonal antibodies are prepared, for example, using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof, or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Coding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Immortalized cell lines that fuse efficiently, support stable high-level expression of antibody by the selected antibody-producing cells, and are sensitive to a medium such as HAT medium may be used. Other immortalized cell lines that may be used are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for making monoclonal antibodies. (Kozbor, J. Immunol., 133:3001 (1984); Brodeur et al., Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (Coding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567. DNA encoding the disclosed monoclonal antibodies can be readily isolated and sequenced using conventional procedures (e.g., by using oligonucleotide probes that are capable of binding specifically to genes encoding the heavy and light chains of murine antibodies). The hybridoma cells producing a disclosed antibody serve as an excellent source of such DNA. Once isolated, the DNA can be placed into expression vectors, which are then transfected into host cells such as Chinese hamster ovary (CHO) cells, Human Embryonic Kidney (HEK) 293 cells, simian COS cells, PER.C6®, NSO cells, SP2/0, YB2/0, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (see U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of a disclosed antibody or can be substituted for the variable domains of one antigen-combining site of a disclosed antibody to create a chimeric bivalent antibody.

The antibodies disclosed herein include fully human antibodies or humanized antibodies. These antibodies are suitable for administration to humans without engendering an immune response by the human against the administered immunoglobulin.

An anti-CD47 antibody is generated, for example, using the procedures described in the Examples provided below. For example, disclosed anti-CD47 antibodies are identified using a modified immunization strategy in mice and subsequent hybridoma generation.

In alternative methods, an anti-CD47 antibody is developed, for example, using phage-display methods using antibodies containing only human sequences. Such approaches are well-known in the art, e.g., in WO92/01047 and U.S. Pat. No. 6,521,404. In this approach, a combinatorial library of phage carrying random pairs of light and heavy chains are screened using natural or recombinant source of cd47 or fragments thereof. In another approach, an anti-CD47 antibody can be produced by a process wherein at least one step of the process includes immunizing a transgenic, non-human animal with human CD47 protein. In this approach, some of the endogenous heavy and/or kappa light chain loci of this xenogenic non-human animal have been disabled and are incapable of the rearrangement required to generate genes encoding immunoglobulins in response to an antigen. In addition, at least one human heavy chain locus and at least one human light chain locus have been stably transfected into the animal. Thus, in response to an administered antigen, the human loci rearrange to provide genes encoding human variable regions immunospecific for the antigen. Upon immunization, therefore, the xenomouse produces B-cells that secrete fully human immunoglobulins.

A variety of techniques are well-known in the art for producing xenogenic non-human animals. For example, see U.S. Pat. Nos. 6,075,181 and 6,150,584. This general strategy was demonstrated with the first XenoMouse™ strains as published in 1994. See Green et al., Nature Genetics 7: 13-21 (1994). See also U.S. Pat. Nos. 6,162,963; 6,150,584; 6,114,598; 6,075,181; and 5,939,598 and Japanese Patent Nos. 3 068 180 B2, 3 068 506 B2, and 3 068 507 B2 and European Patent No., EP 0 463 151 B 1 and International Patent Applications No. WO 94/02602, WO 96/34096, WO 98/24893, WO 00/76310 and related family members.

In an alternative approach, others have utilized a “minilocus” approach in which an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. See e.g., U.S. Pat. Nos. 5,545,806; 5,545,807; 5,591,669; 5,612,205; 5,625,825; 5,625,126; 5,633,425; 5,643,763; 5,661,016; 5,721,367; 5,770,429; 5,789,215; 5,789,650; 5,814,318; 5,877; 397; 5,874,299; 6,023,010; and 6,255,458; and European Patent No. 0 546 073 B1; and International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884 and related family members.

Generation of human antibodies from mice in which, through microcell fusion, large pieces of chromosomes, or entire chromosomes, have been introduced, has also been demonstrated. See European Patent Application No. 773 288 and 843 961. Human anti-mouse antibody (HAMA) responses have led the industry to prepare chimeric or otherwise humanized antibodies. While chimeric antibodies have a human constant region and an immune variable region, it is expected that certain human anti-chimeric antibody (HACA) responses will be observed, particularly in chronic or multi-dose utilizations of the antibody. Thus, this disclosure provides fully human antibodies against CD47 to vitiate or otherwise mitigate concerns and/or effects of HAMA or HACA response.

The production of antibodies with reduced immunogenicity is also accomplished via humanization, chimerization and display techniques using appropriate libraries. It will be appreciated that murine antibodies or antibodies from other species can be humanized or primatized using techniques well known in the art. See e.g., Winter and Harris Immunol Today 14:43 46 (1993) and Wright et al. Crit, Reviews in Immunol. 12125-168 (1992). The antibody of interest may be engineered by recombinant DNA techniques to substitute the CH1, CH2, CH3, hinge domains, and/or the framework domain with the corresponding human sequence (See WO 92102190 and U.S. Pat. Nos. 5,530,101, 5,585,089, 5,693,761, 5,693,792, 5,714,350, and 5,777,085). Also, the use of Ig cDNA for construction of chimeric immunoglobulin genes is known in the art (Liu et al. P.N.A.S. 84:3439 (1987) and J. Immunol. 139:3521 (1987)). mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes may be found in Kabat et al. (1991) Sequences of Proteins of immunological Interest, N.I.H. publication no. 91-3242. Human C region genes are readily available from known clones. The choice of isotype will be guided by the desired effecter functions, such as complement fixation, or activity in antibody-dependent cellular cytotoxicity. Preferred isotypes are IgG_{G}, IgG₂, IgG₃, and IgG₄. Either of the human light chain constant regions, kappa or lambda, may be used. The chimeric, humanized antibody is then expressed by conventional methods.

Antibody fragments, such as Fv, F(ab′)₂ and Fab may be prepared by cleavage of the intact protein, e.g., by protease or chemical cleavage. Alternatively, a truncated gene is designed. For example, a chimeric gene encoding a portion of the F(ab′)₂ fragment would include DNA sequences encoding the CHI domain and hinge region of the H chain, followed by a translational stop codon to yield the truncated molecule.

Consensus sequences of H, L and J regions may be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Expression vectors include plasmids, retroviruses, YACs, EBV derived episomes, and the like. A convenient vector is one that encodes a functionally complete human C_{H} or C_{L} immunoglobulin sequence, with appropriate restriction sites engineered so that any V_{H} or V_{L} sequence can be easily inserted and expressed. In such vectors, splicing usually occurs between the splice donor site in the inserted J region and the splice acceptor site preceding the human C region, and at the splice regions that occur within the human C_{H} exons. Polyadenylation and transcription termination occur at native chromosomal sites downstream of the coding regions. The resulting chimeric antibody may be joined to any strong promoter, including retroviral LTRs, e.g., SV-40 early promoter, (Okayama et al. Mol. Cell. Bio. 3:280 (1983)), Rous sarcoma virus LTR (Gorman et al. P.N.A.S. 79:6777 (1982)), and Moloney murine leukemia virus LTR (Grosschedl et al. Cell 41:885 (1985)). Also, native Ig promoters and the like may be used.

Further, human antibodies or antibodies from other species can be generated through display type technologies, including, without limitation, phage display, retroviral display, ribosomal display, and other techniques, using techniques well known in the art and the resulting molecules can be subjected to additional maturation, such as affinity maturation, as such techniques are well known in the art. Wright et al. Crit, Reviews in Immunol. 12125-168 (1992), Hanes and Pluckthun PNAS USA 94:4937-4942 (1997) (ribosomal display), Parmley and Smith Gene 73:305-318 (1988) (phage display), Scott, TIBS, vol. 17:241-245 (1992), Cwirla et al. PNAS USA 87:6378-6382 (1990), Russel et al. Nucl. Acids Research 21: 1081-1085 (1993), Hoganboom et al. Immunol. Reviews 130:43-68 (1992), Chiswell and McCafferty TIBTECH; 10:80-8A (1992), and U.S. Pat. No. 5,733,743. If display technologies are utilized to produce antibodies that are not human, such antibodies can be humanized as described above.

Using these techniques, antibodies can be generated to CD47 expressing cells, soluble forms of CD47, epitopes or peptides thereof, and expression libraries thereto (See e.g., U.S. Pat. No. 5,703,057) which can thereafter be screened as described above for the activities described herein.

The disclosed anti-CD47 antibodies can be expressed by a vector containing a DNA segment, such as one encoding the single chain antibody described above. Any suitable vector may be used.

These can include vectors, liposomes, naked DNA, adjuvant-assisted DNA, gene gun, catheters, etc. Vectors include chemical conjugates such as described in WO 93/64701, which has targeting moiety (e.g. a ligand to a cellular surface receptor), and a nucleic acid binding moiety (e.g. polylysine), viral vector (e.g. a DNA or RNA viral vector), fusion proteins such as described in PCT/US95/02140 (WO 95/22618) which is a fusion protein containing a target moiety (e.g. an antibody specific for a target cell) and a nucleic acid binding moiety (e.g. a protamine), plasmids, phage, etc. The vectors can be chromosomal, non-chromosomal or synthetic.

Exemplary vectors include viral vectors, fusion proteins and chemical conjugates. Retroviral vectors include Moloney murine leukemia viruses. DNA viral vectors may be used. These vectors include pox vectors such as orthopox or avipox vectors, herpesvirus vectors such as a herpes simplex I virus (HSV) vector (see Geller, A. I. et al., J. Neurochem, 64:487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A. 90:7603 (1993); Geller, A. I., et al., Proc Natl. Acad. Sci USA 87: 1149 (1990), Adenovirus Vectors (see LeGal LaSalle et al., Science, 259:988 (1993); Davidson, et al., Nat. Genet 3:219 (1993); Yang, et al., J. Virol. 69:2004 (1995) and Adeno-associated Virus Vectors (see Kaplitt, M. G., et al., Nat. Genet. 8: 148 (1994).

Pox viral vectors introduce the gene into the cells cytoplasm. Avipox virus vectors result in only a short-term expression of the nucleic acid. Adenovirus vectors, adeno-associated virus vectors and herpes simplex virus (HSV) vectors are preferred for introducing the nucleic acid into neural cells. The adenovirus vector results in a shorter term expression (about 2 months) than adeno-associated virus (about 4 months), which in turn is shorter than HSV vectors. The particular vector chosen will depend upon the target cell and the condition being treated. The introduction can be by standard techniques, e.g. infection, transfection, transduction or transformation. Examples of modes of gene transfer include e.g., naked DNA, CaP04 precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g. adenovirus, HSV) to a desired location. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and may be useful in delivering the vector to the target cell. (See Bobo et al., Proc. Natl. Acad. Sci. USA 91:2076-2080 (1994); Morrison et al., Am. J. Physiol. 266:292-305 (1994)). Other methods that can be used include catheters, intravenous, parenteral, intraperitoneal and subcutaneous injection, and oral or other known routes of administration.

These vectors can be used to express large quantities of antibodies that can be used in a variety of ways. For example, to detect the presence of CD47 in a sample. The antibody can also be used to try to bind to and disrupt CD47- and/or the CD47/SIRPα interaction and CD47/SIRPα-mediated signaling.

Prokaryotic cells, such as E. coli and Bacillus, yeast cells, such as Saccharomyces cerevisiae, and plant cells are provided that bear a vector comprising a nucleic acid encoding a disclosed antibody. In some embodiments, such cell expresses the disclosed antibody. Mammalian cell lines that normally do not bear nucleic acids encoding a disclosed antibody are also provided. These mammalian cells lines bear a vector comprising a nucleic acid encoding a disclosed antibody. In some embodiments, such cell lines express the disclosed antibody.

Techniques can be adapted for the production of single-chain antibodies specific to an antigenic protein of this disclosure (see e.g., U.S. Pat. No. 4,946,778). In addition, methods can be adapted for the construction of Fab expression libraries (see e.g., Huse, et al., 1989 Science 246: 1275-1281) to allow rapid and effective identification of monoclonal Fab fragments with the desired specificity for a protein or derivatives, fragments, analogs or homologs thereof. Antibody fragments that contain the idiotypes to a protein antigen may be produced by techniques known in the art including, but not limited to: (i) an F(ab′)₂ fragment produced by pepsin digestion of an antibody molecule; (ii) an Fab fragment generated by reducing the disulfide bridges of an F(a′)2 fragment; (iii) an Fab fragment generated by the treatment of the antibody molecule with papain and a reducing agent and (iv) Fv fragments.

The disclosed anti-CD47 antibodies also includes Fv, Fab, Fab′ and F(ab′)₂ CD47 fragments, single chain CD47 antibodies, single domain antibodies (e.g., nanobodies or VHHs), bispecific CD47 antibodies, and heteroconjugate CD47 antibodies.

Bispecific antibodies are antibodies that have binding specificities for at least two different antigens. In the present case, one of the binding specificities is for CD47. The second binding target is any other antigen, and advantageously is a cell-surface protein or receptor or receptor subunit.

Methods for making bispecific antibodies are known in the art.

Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full-length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure in which intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized to make antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain connected to a light-chain variable domain by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments with single-chain Fv (sFv) dimers has also been reported. Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen CD47. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcyR), such as FcyRI (CD64), FcyRII (CD32) and FcyRIII (CD 16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate antibodies are also within the scope. Heteroconjugate antibodies are composed of two covalently joined antibodies. Such antibodies have, for example, been proposed to target immune system cells to unwanted cells (see U.S. Pat. No. 4,676,980), and for treatment of HIV infection (see WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

It can be desirable to modify the disclosed antibody regarding effector function, so as to enhance, e.g., the effectiveness of the antibody in treating diseases and disorders associated with aberrant CD47 signaling. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al., J. Exp Med., 176: 1191-1195 (1992) and Shopes, J. Immunol., 148: 2918-2922 (1992)). Alternatively, an antibody can be engineered that has dual Fc regions and can thereby have enhanced complement lysis and ADCC capabilities. (See Stevenson et al., Anti-Cancer Drug Design, 3: 219-230 (1989)).

Immunoconjugates comprising an antibody disclosed herein conjugated to a cytotoxic agent such as a toxin (e.g., an enzymatically active toxin of bacterial, fungal, plant, or animal origin, or fragments thereof), or a radioactive isotope (i.e., a radioconjugate) are also provided.

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available to make radioconjugated antibodies. Examples include 212Bi, 131I, 131In, 90Y, and 186Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

A large variety of possible moieties can be coupled to the resultant antibodies of this disclosure. (See, for example, “Conjugate Vaccines”, Contributions to Microbiology and Immunology, J. M. Cruse and R. E. Lewis, Jr (eds), Carger Press, New York, (1989)).

Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. The preferred binding is, however, covalent binding. Covalent binding can be achieved either by direct condensation of existing side chains or by the incorporation of external bridging molecules. Many bivalent or polyvalent linking agents are useful in coupling protein molecules, such as the disclosed antibodies, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133: 1335-2549 (1984); Jansen et al., Immunological Reviews 62: 185-216 (1982); and Vitetta et al., Science 238: 1098 (1987). Other linkers are described in the literature. (See, e.g., Ramakrishnan, S. et al., Cancer Res. 44:201-208 (1984) describing use of MBS (M-maleimidobenzoyl-N-hydroxysuccinimide ester). See also U.S. Pat. No. 5,030,719, describing use of halogenated acetyl hydrazide derivative coupled to an antibody by way of an oligopeptide linker. Exemplary linkers include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamidojhexanoate (Pierce Chem. Co., Cat #216510); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, particularly, can enhance the stability of carbodimide couplings. Carbodimide couplings (such as EDC) when used in conjunction with sulfo-NHS, forms esters that are more resistant to hydrolysis than the carbodimide coupling reaction alone.

Compositions and Formulations

Pharmaceutical compositions are provided that comprise a disclosed anti-CD47 antibody and a pharmaceutically acceptable excipient (carrier).

Any suitable excipient/carrier may be used. Any suitable route of administration, dosage, and dosing regimen of the disclosed antibody or pharmaceutical composition comprising a disclosed antibody may be used.

The disclosed anti-CD47 antibodies (also referred to herein as “active compounds”), and derivatives, fragments, analogs and homologs thereof, can be incorporated into pharmaceutical compositions suitable for administration to a patient. Principles and considerations involved in preparing such compositions, and guidance in the choice of components are provided, for example, in Remington's Pharmaceutical Sciences: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

Where antibody fragments are used, the smallest inhibitory fragment that specifically binds to the binding domain of the target protein may be used. For example, based upon the variable-region sequences of an antibody, peptide molecules can be designed that retain the ability to bind the target protein sequence. Such peptides can be synthesized chemically and/or produced by recombinant DNA technology. (see, e.g., Marasco et al., Proc. Natl. Acad. Sci. USA, 90: 7889-7893 (1993)).

As used herein, the term “pharmaceutically acceptable excipient” is intended to include any and all solvents, dispersion media, coatings, antibacterial and antifungal agents, isotonic and absorption delaying agents, and the like, compatible with pharmaceutical administration. Suitable carriers/excipients are described in the most recent edition of Remington's Pharmaceutical Sciences. Examples of such carriers or diluents include, but are not limited to, water, saline, ringer's solutions, dextrose solution, and 5% human serum albumin.

Liposomes and non-aqueous vehicles such as fixed oils may also be used to formulate a composition comprising a disclosed anti-CD47 antibody. The use of such media and agents for pharmaceutically active substances is well known in the art. Except insofar as any conventional media or agent is incompatible with the disclosed anti-CD47 antibody, use thereof in the compositions is contemplated.

The disclosed antibodies can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545.

Liposomes with enhanced circulation time are disclosed in U.S. Pat. No. 5,013,556. Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of a disclosed antibody can be conjugated to the liposomes as described in Martin et al., J. Biol. Chem., 257: 286-288 (1982) via a disulfide-interchange reaction.

The formulations to be used for in vivo administration must be sterile. This is readily accomplished by, for example, filtration through sterile filtration membranes.

A pharmaceutical composition is formulated to be compatible with its intended route of administration. Examples of routes of administration of a pharmaceutical composition comprising one or more disclosed anti-CD47 antibody (with or without additional agents, as well as a pharmaceutical composition comprising an additional agent) include parenteral, e.g., intravenous (IV), intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier/excipient can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents are included, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the disclosed anti-CD47 antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the disclosed anti-CD47 antibody can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the disclosed anti-CD47 antibody is delivered in the form of an aerosol spray from pressured container or dispenser which contains a suitable propellant, e.g., a gas such as carbon dioxide, or a nebulizer.

Systemic administration can also be by transmucosal or transdermal means.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The disclosed anti-CD47 antibody can also be prepared in the form of suppositories (e.g., with conventional suppository bases such as cocoa butter and other glycerides) or retention enemas for rectal delivery.

In some embodiments, the disclosed anti-CD47 antibody is prepared with carriers that will protect the disclosed anti-CD47 antibody against rapid elimination from the body, such as sustained/controlled release formulations, including implants and microencapsulated delivery systems. Biodegradable, biocompatible polymers can be used, such as ethylene vinyl acetate, poly anhydrides, polyglycolic acid, collagen, polyorthoesters, and polylactic acid. Methods for preparation of such formulations will be apparent to those skilled in the art.

For example, the active ingredients can be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The materials can also be obtained commercially from Alza Corporation and Nova Pharmaceuticals, Inc. Liposomal suspensions (including liposomes targeted to infected cells with monoclonal antibodies to viral antigens) and can also be used as pharmaceutically acceptable carriers. These can be prepared according to methods known to those skilled in the art, for example, as described in U.S. Pat. No. 4,522,811.

Oral or parenteral compositions may be formulated in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms are dictated by and directly dependent on the unique characteristics of the active compound and the particular therapeutic effect to be achieved, and the limitations inherent in the art of compounding such an active compound for the treatment of individuals. The pharmaceutical compositions can be included in a container, pack, or dispenser together with instructions for administration.

These pharmaceutical compositions can be included in kits, such as, for example, diagnostic kits.

Combination Therapy

In some embodiments, the anti-CD47 antibodies described herein are administered to a patient with one or more additional agents. Any suitable agent is contemplated.

Suitable additional agents include current pharmaceutical and/or surgical therapies for an intended application, such as, for example, cancer. For example, the anti-CD47 antibodies can be administered with one or more additional chemotherapeutic or anti-neoplastic agents. Alternatively, the additional chemotherapeutic agent is radiotherapy. In some embodiments, the chemotherapeutic agent is a cell death-inducing agent. In some embodiments, the chemotherapeutic agent induces a loss of phospholipid asymmetry across the plasma membrane, for example causes cell surface exposure of phosphatidylserine (PS). In some embodiments, the chemotherapeutic agent induces endoplasmic reticulum (ER) stress. In some embodiments, the chemotherapeutic agent is a proteasome inhibitor. In some embodiments, the chemotherapeutic agent induces the translocation of ER proteins to the cell surface. In some embodiments, the chemotherapeutic agent induces the translocation and cell surface exposure of calreticulin.

In some embodiments, the anti-CD47 antibody and additional agent are formulated into a single therapeutic composition, and a disclosed anti-CD47 antibody and additional agent are administered simultaneously. Alternatively, a disclosed anti-CD47 antibody and additional agent are separate from each other, e.g., each is formulated into a separate therapeutic composition, and the disclosed anti-CD47 antibody and the additional agent are administered simultaneously, or at different times during a treatment regimen. For example, the anti-CD47 antibody is administered prior to or subsequent to the administration of the additional agent or the anti-CD47 antibody and the additional agent are administered in an alternating fashion. The disclosed anti-CD47 antibody and additional agent may be administered in single doses or in multiple doses.

The formulation can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

In certain embodiments, the active compounds (which includes a disclosed anti-CD47 antibody) are administered in combination therapy, i.e., combined with other agents, e.g., therapeutic agents, that are useful for treating pathological conditions or disorders, such as various forms of cancer, autoimmune disorders and inflammatory diseases. The term “in combination” in this context means that the agents are given substantially contemporaneously, either simultaneously or sequentially. If given sequentially, at the onset of administration of the second compound, the first of the two compounds may still be detectable at effective concentrations at the site of treatment.

For example, the combination therapy can include one or more disclosed anti-CD47 antibodies coformulated with, and/or coadministered with, one or more additional therapeutic agents, e.g., one or more cytokine and growth factor inhibitors, immunosuppressants, anti-inflammatory agents, metabolic inhibitors, enzyme inhibitors, and/or cytotoxic or cytostatic agents, as described in more detail below. Such combination therapies may advantageously utilize lower dosages of the administered therapeutic agents, thus avoiding possible toxicities or complications associated with the various monotherapies.

In other embodiments, the disclosed anti-CD47 antibodies are used as vaccine adjuvants against autoimmune disorders, inflammatory diseases, etc. The combination of adjuvants for treatment of these types of disorders are suitable for use in combination with a wide variety of antigens from targeted self-antigens, i.e., autoantigens, involved in autoimmunity, e.g., myelin basic protein; inflammatory self-antigens, e.g., amyloid peptide protein, or transplant antigens, e.g., alloantigens. The antigen may comprise peptides or polypeptides derived from proteins, as well as fragments of any of the following: saccharides, proteins, polynucleotides or oligonucleotides, autoantigens, amyloid peptide protein, transplant antigens, allergens, or other macromolecular components. In some instances, more than one antigens are included in the antigenic composition.

Gene therapy vectors known in the art may also be employed with the vectors bearing a nucleic acid encoding a disclosed anti-CD47 antibody and the antibody may be expressed by suitable expression system inside a human patient.

Methods of Using the Disclosed Anti-CD47 Antibodies and Compositions

The anti-CD47 antibodies described herein are useful in treating, delaying the progression of, preventing relapse of, or alleviating a symptom of a cancer or other neoplastic condition; such as, for example, treating hematological malignancies and/or tumors.

This disclosure provides a method of treating, delaying the progression of, preventing relapse of, or alleviating a symptom of a cancer or other neoplastic condition in a human patient with cancer or other neoplastic condition. The method comprises administering to the patient a therapeutically effective amount of a disclosed anti-CD47 antibody or administering to the patient a therapeutically effective amount of a pharmaceutical composition comprising the disclosed anti-CD47 antibody and a pharmaceutical excipient and/or carrier. The method further comprises administering one or more additional agents to the patient. In certain embodiments, the additional agent(s) is a therapeutic agent. In certain further embodiments, the therapeutic agent(s) is an anti-cancer agent.

In certain embodiments, the CD47 antibodies described herein are used in treating CD47⁺ tumors.

In some embodiments, the disclosed anti-CD47 antibodies are used in treating non-Hodgkin's lymphoma (NHL), acute lymphocytic leukemia (ALL), acute myeloid leukemia (AML), chronic lymphocytic leukemia (CLL), chronic myelogenous leukemia (CML), multiple myeloma (MM), breast cancer, ovarian cancer, head and neck cancer, bladder cancer, melanoma, colorectal cancer, pancreatic cancer, lung cancer, leiomyoma, leiomyosarcoma, glioma, glioblastoma. Solid tumors include, for example, breast tumors, ovarian tumors, lung tumors, pancreatic tumors, prostate tumors, melanoma tumors, colorectal tumors, lung tumors, head and neck tumors, bladder tumors, esophageal tumors, liver tumors, and kidney tumors.

As used herein, “hematological cancer” refers to a cancer of the blood, and includes leukemia, lymphoma and myeloma among others.

“Leukemia” refers to a cancer of the blood in which too many white blood cells that are ineffective in fighting infection are made, thus crowding out the other parts that make up the blood, such as platelets and red blood cells. It is understood that cases of leukemia are classified as acute or chronic. Certain forms of leukemia include, by way of non-limiting example, acute lymphocytic leukemia (ALL); acute myeloid leukemia (AML); chronic lymphocytic leukemia (CLL); chronic myelogenous leukemia (CML); Myeloproliferative disorder/neoplasm (MPDS); and myelodysplasia syndrome.

“Lymphoma” may refer to a Hodgkin's lymphoma, both indolent and aggressive non-Hodgkin's lymphoma, Burkitt's lymphoma, and follicular lymphoma (small cell and large cell), among others.

Myeloma may refer to multiple myeloma (MM), giant cell myeloma, heavy-chain myeloma, and light chain or Bence-Jones myeloma.

In other aspects, methods are provided for alleviating a symptom of a cancer or other neoplastic condition by administering to a subject in need thereof one or more disclosed anti-CD47 antibodies. The antibody does not cause a significant level of hemagglutination of red blood cells after administration. The antibody is administered in an amount sufficient to alleviate the symptom of the cancer or other neoplastic condition in the subject.

Administration of therapeutic entities would include suitable excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies, provided that the active ingredient in the formulation is not inactivated by the formulation and the formulation is physiologically compatible and tolerable with the route of administration. See also Baldrick P. Regul. Toxicol Pharmacol. 32(2):210-8 (2000), Wang W. Int. J. Pharm. 203(1-2): 1-60 (2000), Charman W N J Pharm Sci. 89(8):967-78 (2000), Powell et al. PDA J Pharm Sci Technol. 52:238-311 (1998) and the citations therein for additional information related to formulations, excipients and carriers well known to pharmaceutical chemists.

Symptoms associated with cancers and other neoplastic disorders include, for example, inflammation, fever, general malaise, fever, pain, often localized to the inflamed area, loss of appetite, weight loss, edema, headache, fatigue, rash, anemia, muscle weakness, muscle fatigue and abdominal symptoms such as, for example, abdominal pain, diarrhea or constipation.

A therapeutically effective amount of a disclosed anti-CD47 antibody relates generally to the amount needed to achieve a therapeutic objective. As noted above, this may be a binding interaction between the antibody and its target antigen that, in certain cases, interferes with the functioning of the target. A therapeutic objective includes, for example, cancer regression, cancer cure, alleviation of one or more symptoms of cancer, and delaying death of the patient by at least one day.

The amount of a disclosed anti-CD47 antibody required to be administered depends on the binding affinity of the antibody for its specific antigen, and will also depend on the rate at which an administered antibody is depleted from the free volume other subject to which it is administered. Common ranges for therapeutically effective dosing of a disclosed anti-CD47 antibody may be, by way of nonlimiting example, from about 0.1 mg/kg body weight to about 100 mg/kg body weight. In some embodiments, a disclosed antibody is administered to a subject a dose of 0.1 mg/kg, 0.5 mg/kg, 1 mg/kg, 2 mg/kg, 5 mg/kg, 10 mg/kg, 15 mg/kg, 20 mg/kg, 25 mg/kg, 30 mg/kg, 50 mg/kg, 75 mg/kg, 100 mg/kg, or greater. Common dosing frequencies may range, for example, from twice daily to once a week.

Efficaciousness of treatment is determined in association with any known method for diagnosing or treating the disease, such as cancer. Alleviation of one or more symptoms of the disease indicates that the antibody confers a clinical benefit and is thus therapeutically effective.

In certain embodiments, disclosed anti-CD47 antibodies, which include a monoclonal antibody, may be used as therapeutic agents. Such agents may be employed to diagnose, prognose, monitor, treat, alleviate, and/or prevent a disease or pathology associated with aberrant CD47 expression, activity and/or signaling in a subject. A therapeutic regimen is carried out by identifying a subject, e.g., a human patient suffering from (or at risk of developing) a disease or disorder associated with aberrant CD47 expression, activity and/or signaling, e.g., a cancer or other neoplastic disorder, using standard methods. An antibody preparation, preferably one having high specificity and high affinity for its target antigen, is administered to the subject and will generally have an effect due to its binding with the target. Administration of the antibody may abrogate or inhibit or interfere with the expression, activity and/or signaling function of the target (e.g., CD47). Administration of the antibody may abrogate or inhibit or interfere with the binding of the target (e.g., CD47) with an endogenous ligand (e.g., SIRPα) to which it naturally binds. For example, the antibody binds to the target and modulates, blocks, inhibits, reduces, antagonizes, neutralizes, or otherwise interferes with CD47 expression, activity and/or signaling.

Diseases or disorders related to aberrant CD47 expression, activity and/or signaling include, by way of non-limiting example, hematological cancer and/or solid tumors. Hematological cancers include, e.g., leukemia, lymphoma and myeloma. Certain forms of leukemia include, by way of non-limiting example, acute lymphocytic leukemia (ALL); acute myeloid leukemia (AML); chronic lymphocytic leukemia (CLL); chronic myelogenous leukemia (CML); Myeloproliferative disorder/neoplasm (MPDS); and myelodysplasia syndrome. Certain forms of lymphoma include, by way of non-limiting example, Hodgkin's lymphoma, both indolent and aggressive non-Hodgkin's lymphoma, Burkitt's lymphoma, and follicular lymphoma (small cell and large cell). Certain forms of myeloma include, by way of non-limiting example, multiple myeloma (MM), giant cell myeloma, heavy-chain myeloma, and light chain or Bence-Jones myeloma. Solid tumors include, e.g., breast tumors, ovarian tumors, lung tumors, pancreatic tumors, prostate tumors, melanoma tumors, colorectal tumors, lung tumors, head and neck tumors, bladder tumors, esophageal tumors, liver tumors, and kidney tumors.

Methods for the screening of antibodies that possess the desired specificity include, but are not limited to, enzyme linked immunosorbent assay (ELISA) and other immunologically mediated techniques known within the art.

In other embodiments, antibodies directed against CD47 may be used in methods known within the art relating to the localization and/or quantitation of CD47 (e.g., for use in measuring levels of CD47 and/or both CD47 and SIRPα within appropriate physiological samples, for use in diagnostic methods, for use in imaging the protein, and the like).

In other embodiments, an anti-CD47 antibody can be used to isolate a CD47 polypeptide, by standard techniques, such as immunoaffinity, chromatography or immunoprecipitation. Antibodies directed against the CD47 protein (or a fragment thereof) can be used diagnostically to monitor protein levels in tissue as part of a clinical testing procedure, e.g., to, for example, determine the efficacy of a given treatment regimen.

Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

In some embodiments, the antibody contains a detectable label. Antibodies may be, for example, polyclonal, or monoclonal. An intact antibody, or a fragment thereof (e.g., Fab, scFv, or F(ab′)₂) is used. The term “labeled”, regarding the probe or antibody, is intended to encompass direct labeling of the probe or antibody by coupling (i.e., physically linking) a detectable substance to the probe or antibody, and indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin.

The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

Design and Generation of Other Therapeutics

Based on the activity of the antibodies produced and characterized herein with respect to CD47, the design of other therapeutic modalities beyond antibody moieties is facilitated. Such modalities include, without limitation, advanced antibody therapeutics, such as bispecific antibodies, immunotoxins, and radiolabeled therapeutics, generation of peptide therapeutics, gene therapies, particularly intrabodies, antisense therapeutics, and small molecules.

Bispecific antibodies can be generated that comprise (i) two antibodies, one with a specificity to CD47 and another to a second molecule, that are conjugated together, (ii) a single antibody that has one chain specific to CD47 and a second chain specific to a second molecule, or (iii) a single chain antibody that has specificity to CD47 and a second molecule. Such bispecific antibodies are generated using techniques that are well known (see e.g., Fanger et al. Immunol Methods 4:72-81 (1994) and Wright et al. Crit, Reviews in Immunol. 12125-168 (1992), Traunecker et al. Int. J. Cancer (Suppl.) 7:51-52 (1992)).

Antibodies can be modified to act as immunotoxins utilizing techniques that are well known in the art. See e.g., Vitetta Immunol Today 14:252 (1993). See also U.S. Pat. No. 5,194,594.

Preparation of radiolabeled antibodies, such modified antibodies can also be readily prepared utilizing techniques that are well known in the art. See e.g., Junghans et al. in Cancer Chemotherapy and Biotherapy 655-686 (2d edition, Chafner and Longo, eds., Lippincott Raven (1996)). See also U.S. Pat. Nos. 4,681,581, 4,735,210, 5,101,827, 5,102,990 (RE 35,500), 5,648,471, and 5,697,902.

Each of immunotoxins and radiolabeled molecules would be likely to kill cells expressing CD47.

Through the utilization of structural information related to CD47 and antibodies thereto, such as the disclosed anti-CD47 antibodies or screening of peptide libraries, therapeutic peptides can be generated that are directed against CD47. Design and screening of peptide therapeutics is discussed in Houghten et al. Biotechniques 13:412-421 (1992), Houghten PNAS USA 82:5131-5135 (1985), Pinalla et al. Biotechniques 13:901-905 (1992), Blake and Litzi-Davis BioConjugate Chem. 3:510-513 (1992).

Immunotoxins, radiolabeled molecules and peptidic moieties can also be prepared. Assuming that the CD47 molecule (or a form, such as a splice variant or alternate form) is functionally active in a disease process, it will also be possible to design gene and antisense therapeutics thereto through conventional techniques. Such modalities can be utilized for modulating the function of CD47. The disclosed anti-CD47 antibodies facilitate design and use of functional assays related thereto. A design and strategy for antisense therapeutics is discussed in detail in International Patent Application No. WO 94/29444. Design and strategies for gene therapy are well known. The use of gene therapeutic techniques involving intrabodies is also provided. See e.g., Chen et al. Human Gene Therapy 5:595-601 (1994) and Marasco Gene Therapy 4: 11-15 (1997). General design of and considerations related to gene therapeutics is also discussed in International Patent Application No. WO 97/38137.

Knowledge gleaned from the structure of the CD47 molecule and its interactions with other molecules, such as SIRPα and/or the disclosed anti-CD47 antibodies o, and others can be utilized to rationally design additional therapeutic modalities. In this regard, rational drug design techniques such as X-ray crystallography, computer-aided (or assisted) molecular modeling (CAMM), quantitative or qualitative structure-activity relationship (QSAR), and similar technologies can be utilized to focus drug discovery efforts. Rational design allows prediction of protein or synthetic structures which can interact with the molecule or specific forms thereof which can be used to modify or modulate the activity of IL-6Rc. Such structures can be synthesized chemically or expressed in biological systems. This approach has been reviewed in Capsey et al. Genetically Engineered Human Therapeutic Drugs (Stockton Press, NY (1988)). Further, combinatorial libraries can be designed and synthesized and used in screening programs, such as high throughput screening efforts.

Screening Methods

In certain embodiments, a method is provided comprising identifying a compound that interferes with the binding of CD47 to SIRPα by identifying a compound that disrupts the interaction of CD47 and SIRPα. These methods (also referred to herein as “screening assays”) are used for identifying modulators, i.e., candidate or test compounds or agents (e.g., peptides, peptidomimetics, small molecules or other drugs) that modulate or otherwise interfere with the binding of CD47 to SIRPα, or candidate or test compounds or agents that modulate or otherwise interfere with the signaling function of CD47 and/or CD47-SIRPα. Also provided are methods of identifying compounds useful to treat disorders associated with aberrant CD47 and/or CD47-SIRPα expression, activity and/or signaling. The screening methods can include those known or used in the art or those described herein. For example, CD47 can be immobilized on a microtiter plate and incubated with a candidate or test compound, e.g., a CD47 antibody, in the presence of SIRPα. Subsequently, bound SIRPα can be detected using a secondary antibody, and absorbance can be detected on a plate reader.

In certain embodiments, a method is provided comprising identifying a compound that promotes phagocytosis of tumor cells to macrophages. These methods can include those known or used in the art or those described herein. For example, macrophages are incubated with labeled tumor cells in the presence of a candidate compound, e.g., a CD47 antibody. After a period of time, the macrophages can be observed for internalization of the tumor label to identify phagocytosis. Additional details regarding these methods, e.g., SIRPα blocking assays and phagocytosis assays, are provided in the Examples.

In certain embodiments, assays are provided comprising screening candidate or test compounds that modulate the signaling function of CD47. The test compounds can be obtained using any of the numerous approaches in combinatorial library methods known in the art, including biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. (See, e.g., Lam, 1997. Anticancer Drug Design 12: 145).

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays disclosed herein or known in the art.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422.

Libraries of compounds may be presented in solution (see e.g., Houghten, 1992. Biotechniques 13: 412-421), or on beads (see Lam, 1991. Nature 354: 82-84), on chips (see Fodor, 1993. Nature 364: 555-556), bacteria (see U.S. Pat. No. 5,223,409), spores (see U.S. Pat. No. 5,233,409), plasmids (see Cull, et al., 1992. Proc. Natl. Acad. Sci. USA 89: 1865-1869) or on phage (see Scott and Smith, 1990. Science 249: 386-390 and U.S. Pat. No. 5,233,409).

In certain embodiments, a method is provided comprising identifying a compound that disrupts an anti-CD47/CD47 complex. In some embodiments, a candidate compound is introduced to an antibody-antigen complex and determining whether the candidate compound disrupts the antibody-antigen complex, wherein a disruption of this complex indicates that the candidate compound modulates the signaling function of CD47 and/or the interaction between CD47 and SIRPα. In other embodiments, a soluble CD47 and/or both CD47 and SIRPα protein are provided and exposed to at least one neutralizing monoclonal antibody. Formation of an antibody-antigen complex is detected, and one or more candidate compounds are introduced to the complex. If the antibody-antigen complex is disrupted following introduction of the one or more candidate compounds, the candidate compounds is useful to treat disorders associated with aberrant CD47 and/or CD47-SIRPα signaling.

Determining the ability of the test compound to interfere with or disrupt the antibody-antigen complex can be accomplished, for example, by coupling the test compound with a radioisotope or enzymatic label such that binding of the test compound to the antigen or biologically-active portion thereof can be determined by detecting the labeled compound in a complex. For example, test compounds can be labeled with I¹²⁵, S³⁵, C¹⁴, or H³, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product.

In some embodiments, the assay comprises contacting an antibody-antigen complex with a test compound, and determining the ability of the test compound to interact with the antigen or otherwise disrupt the existing antibody-antigen complex. Determining the ability of the test compound to interact with the antigen and/or disrupt the antibody-antigen complex comprises determining the ability of the test compound to preferentially bind to the antigen or a biologically-active portion thereof, as compared to the antibody.

In other embodiments, the assay comprises contacting an antibody-antigen complex with a test compound and determining the ability of the test compound to modulate the antibody-antigen complex. Determining the ability of the test compound to modulate the antibody-antigen complex can be accomplished, for example, by determining the ability of the antigen to bind to or interact with the antibody, in the presence of the test compound.

In any of the screening methods disclosed herein, the antibody may be a neutralizing antibody, which modulates or otherwise interferes with CD47 activity and/or signaling.

The screening methods disclosed herein may be performed as a cell-based assay or as a cell-free assay. The cell-free assays are amenable to use of either the soluble form or the membrane-bound form of CD47 and fragments thereof. In the case of cell-free assays comprising the membrane-bound form of CD47, a solubilizing agent may be used such that the membrane-bound form of the proteins is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)n, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In certain embodiments, either the antibody or the antigen is immobilized to facilitate separation of complexed from uncomplexed forms of one or both following introduction of the candidate compound, and to accommodate automation of the assay. Observation of the antibody-antigen complex in the presence and absence of a candidate compound can be accomplished in any vessel suitable for containing the reactants. Examples of such vessels include microtiter plates, test tubes, and micro-centrifuge tubes. In some embodiments, a fusion protein that adds a domain that allows one or both of the proteins to be bound to a matrix can be provided. For example, GST-antibody fusion proteins or GST-antigen fusion proteins can be adsorbed onto glutathione sepharose beads (Sigma Chemical, St. Louis, Mo.) or glutathione derivatized microtiter plates, that are then combined with the test compound, and the mixture is incubated under conditions conducive to complex formation (e.g., at physiological conditions for salt and pH).

Following incubation, the beads or microtiter plate wells are washed to remove any unbound components, the matrix immobilized in the case of beads, complex determined either directly or indirectly. Alternatively, the complexes can be dissociated from the matrix, and the level of antibody-antigen complex formation can be determined using standard techniques.

Other techniques for immobilizing proteins on matrices can also be used in the screening assays. For example, either the antibody (e.g., an antibody having a variable heavy chain selected from SEQ ID NOs: 349, 351, 353, 355, 357, 359, 361-373, 380-383, 388-392 and a variable light chain selected from SEQ ID NOs: 350, 352, 354, 356, 358, 360, 374-379, 384-387 and 393-396) or the antigen (e.g. CD47 protein) can be immobilized utilizing conjugation of biotin and streptavidin. Biotinylated antibody or antigen molecules can be prepared from biotin-NHS (N-hydroxy-succinimide) using techniques well-known within the art (e.g., biotinylation kit, Pierce Chemicals, Rockford, 1L, USA), and immobilized in the wells of streptavidin-coated 96 well plates (Pierce Chemicals). Alternatively, other antibodies reactive with the antibody or antigen of interest, but which do not interfere with the formation of the antibody-antigen complex of interest, can be derivatized to the wells of the plate, and unbound antibody or antigen trapped in the wells by antibody conjugation. Methods for detecting such complexes, in addition to those described above for the GST-immobilized complexes, include immunodetection of complexes using such other antibodies reactive with the antibody or antigen.

The compounds identified by these screening assays are also provided.

Diagnostic and Prophylactic Formulations

The disclosed anti-CD47 antibodies may be used in diagnostic and prophylactic formulations. In some embodiments, a disclosed anti-CD47 antibody is administered to patients that are at risk of developing one or more of the aforementioned diseases, such as for example, without limitation, cancer or other neoplastic condition. A patient's or organ's predisposition to one or more of the aforementioned cancers or other neoplastic conditions can be determined using genotypic, serological or biochemical markers.

In other embodiments, the disclosed anti-CD47 antibody is administered to human individuals diagnosed with a clinical indication associated with one or more of the aforementioned diseases, such as for example, without limitation, cancer or other neoplastic condition. Upon diagnosis, the disclosed anti-CD47 antibody is administered to mitigate or reverse the effects of the clinical indication associated with one or more of the aforementioned diseases.

The disclosed anti-CD47 antibodies are also useful in the detection of CD47 and/or SIRPα in patient samples and accordingly are useful as diagnostics. For example, the disclosed anti-CD47 antibodies may be used in in vitro assays, e.g., ELISA, to detect CD47 and/or SIRPα levels in a patient sample.

In some embodiments, a disclosed anti-CD47 antibody is immobilized on a solid support (e.g., the well(s) of a microtiter plate). The immobilized antibody serves as a capture antibody for any CD47 and/or SIRPα that may be present in a test sample. Prior to contacting the immobilized antibody with a patient sample, the solid support is rinsed and treated with a blocking agent such as milk protein or albumin to prevent nonspecific adsorption of the analyte.

Subsequently the wells are treated with a test sample suspected of containing the antigen, or with a solution containing a standard amount of the antigen. Such a sample is, e.g., a serum sample from a subject suspected of having levels of circulating antigen considered to be diagnostic of a pathology. After rinsing away the test sample or standard, the solid support is treated with a second antibody that is detectably labeled. The labeled second antibody serves as a detecting antibody. The level of detectable label is measured, and the concentration of CD47 and/or SIRPα in the test sample is determined by comparison with a standard curve developed from the standard samples.

Based on the results obtained using the disclosed anti-CD47 antibodies in an in vitro diagnostic assay, it is possible to stage a disease (e.g., a clinical indication associated with ischemia, an autoimmune or inflammatory disorder) in a subject based on expression levels of CD47 and/or SIRPα. For a given disease, samples of blood are taken from subjects diagnosed as being at various stages in the progression of the disease, and/or at various points in the therapeutic treatment of the disease. Using a population of samples that provides statistically significant results for each stage of progression or therapy, a range of concentrations of the antigen that may be considered characteristic of each stage is designated.

Citation of publications and patent documents is not intended as an admission that any is pertinent prior art, nor does it constitute any admission as to the contents or date of the same.

The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1. Generation and Selection of Anti-CD47 Antibodies

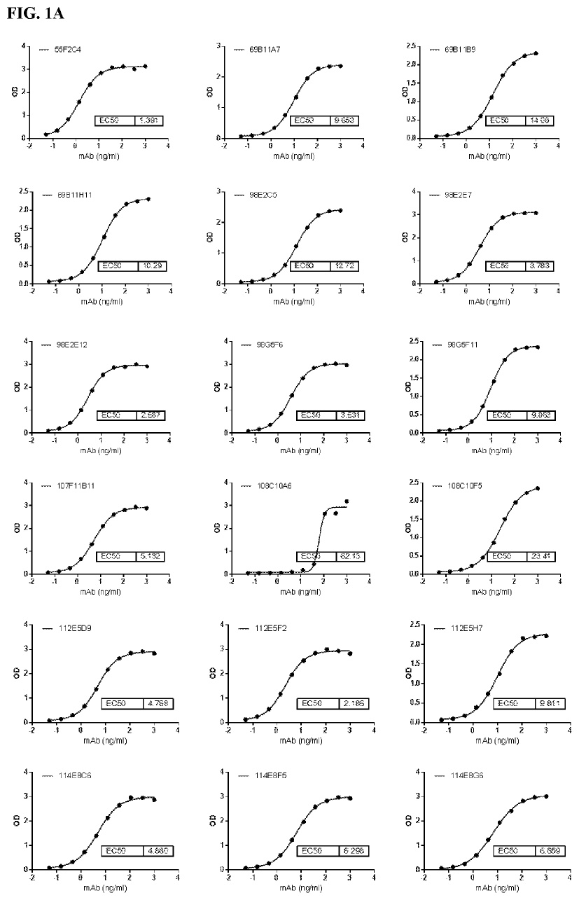

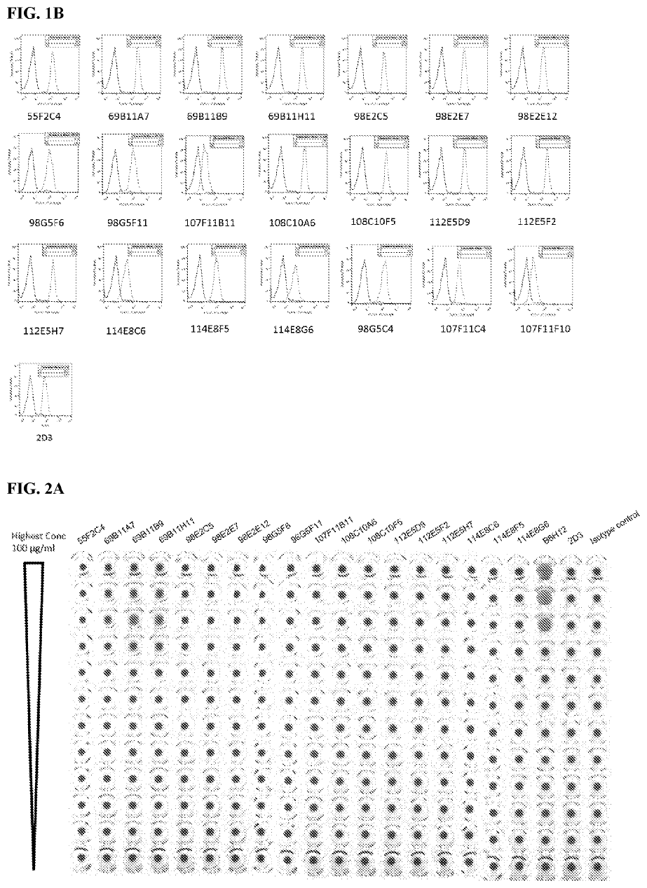

Anti-CD47 antibodies were generated by immunizing mice with an extra-cellular domain of recombinant human CD47 with an Fc tag (CD47-Fc). 200 μl of equally premixed 50 μg of CD47-Fc protein and Complete Freud Adjuvant (Sigma-Aldrich) were used to immunize mice subcutaneously. Subsequently, these mice were boosted with 200 μl of equally premixed 25 μg of CD47-Fc protein and Incomplete Freud Adjuvant (Sigma-Aldrich), alternating intraperitoneally and subcutaneously, every two weeks for three times. Four days before fusion, the selected mouse was boosted with 25 μg CD47-Fc without adjuvant intraperitoneally. Following the immunization schedule, lymph nodes form all mice were harvested and dissociated, thereby enabling B-cell isolation and subsequent fusion to mouse myeloma cells. Both hybridoma supernatants and purified murine CD47 monoclonal antibodies were screened for binding to CD47 by ELISA (FIG. 1A) and by flow cytometry on Raji cells (FIG. 1B).

### Example 2. In Vitro Characterization of Murine CD47 Antibodies

Hemagglutination Activity of Anti-CD47 Antibodies

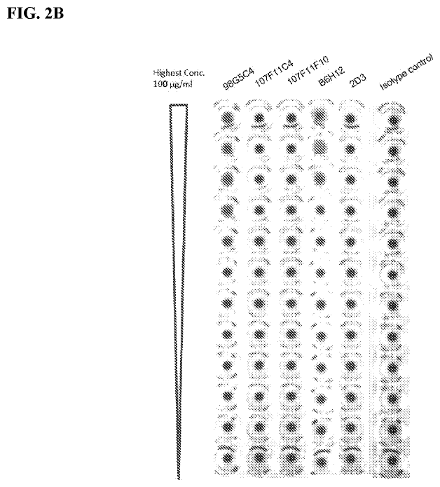

To evaluate the hemagglutination capacity of murine antibodies, human RBCs were incubated with a dose range of anti-CD47 antibodies or control in a 96-well plate. Evidence of hemagglutination was demonstrated by the presence of non-settled RBCs, appearing as a haze compared to a punctate dot of non-hemagglutinated RBCs. As shown in FIG. 2A and FIG. 2B, most of the murine antibodies did not exhibit hemagglutination activity at any of the concentration tested despite presence of B6H12 antibody, which is known to cause hemagglutination.

Briefly, human red blood cells (RBCs) were isolated from healthy donors in the day of experiment. The cells were washed with PBS several times and the RBCs were diluted to 200 million cells per ml in assay buffer. 50 μl of the cell solution and 2×antibody solution were placed in a round bottom 96-well plate, and mixed gently. After incubating the plate in 37° C./5% CO₂ incubator for 2 hours, photos were taken of the assay plates.

Binding to Human CD47

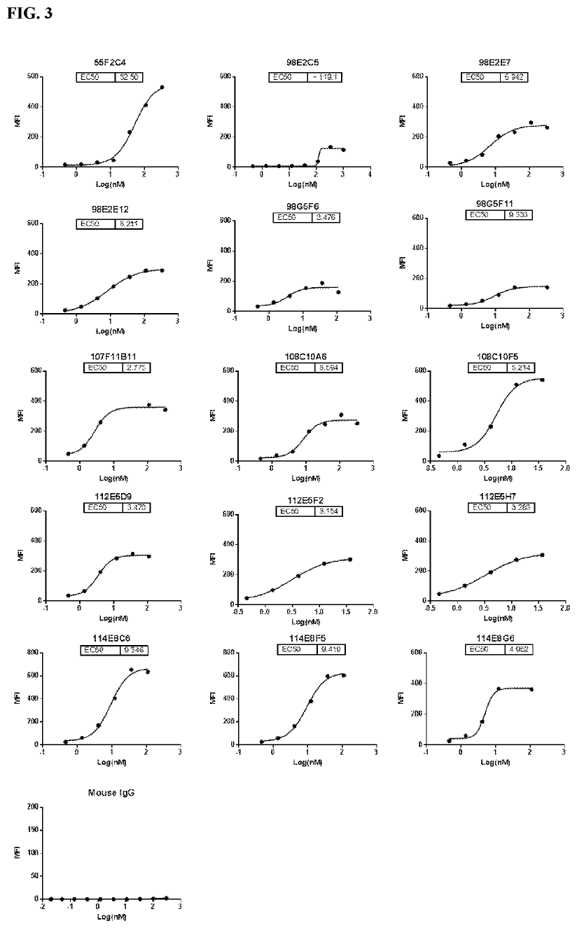

The ability of murine CD47 antibodies to bind to human CD47 was assessed. As shown in FIG. 3, murine CD47 antibodies binds to human CD47.

Binding to cell-expressed human CD47 was evaluated on human CD47 over-expressing CHO cells (GenScript, Cat #M00581). Anti-CD47 antibody dilution series was prepared in FACS buffer. CHO-K1/human CD47 cells were transferred to a 96-well U-bottom plate and anti-CD47 antibody dilution was added. After 30 minutes incubation at 4° C., cells were washed twice with FACS buffer, goat anti-Human IgG (H+L) Secondary Antibody, Alexa Fluor® 647 (Thermo Fisher, cat #A-21445), was added or PE goat anti-mouse IgG (minimal cross-reactivity) Antibody (BioLegend, Cat #405307), was added, incubate at 4° C. for 30 minutes. The cells were then washed twice with wash buffer. Cells were analyzed for binding by FACSCalibur (BD Bioscience, San Jose, Calif.) and Flowjo software.

Binding to Cynomolgus Monkey CD47

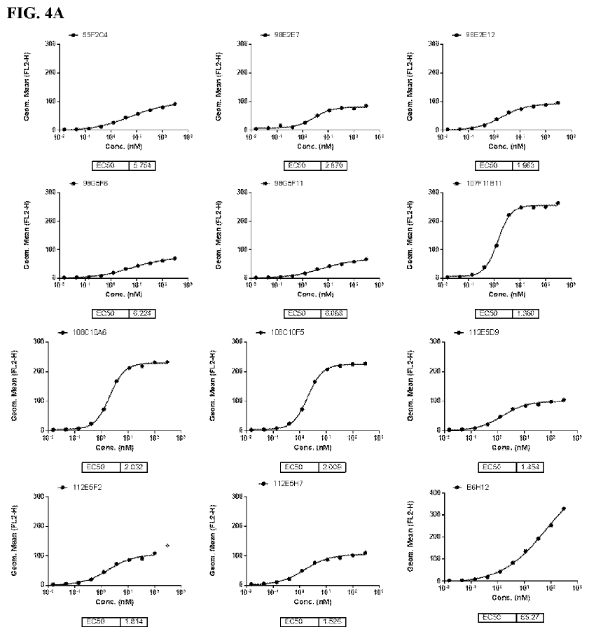

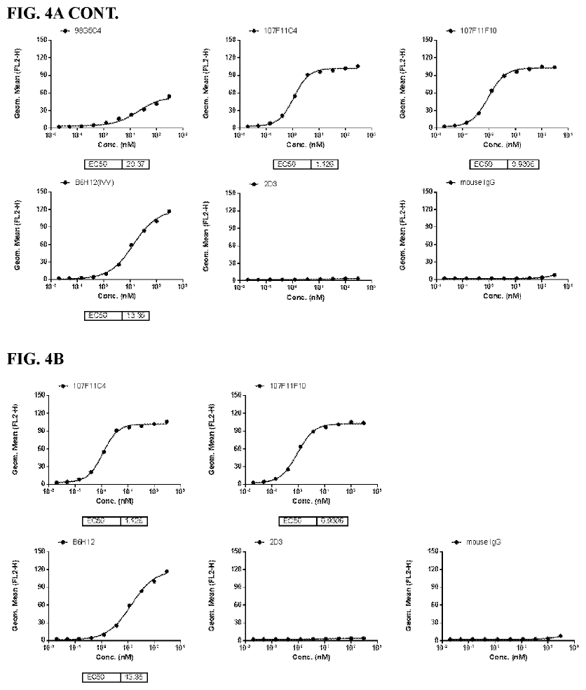

The ability of murine CD47 antibodies to bind to cynomolgus (cyno) monkey CD47 was assessed. As shown in FIG. 4A and FIG. 4B, murine CD47 antibodies binds to cyno CD47.

Briefly, binding to cell-expressed cyno CD47 was evaluated on cynomolgus monkey CD47 over-expressing CHO cells. Anti-CD47 antibody dilution series was prepared in FACS buffer. CHO-K1/cynomolgus monkey CD47 cell were transferred to a 96-well U-bottom plate and add CD47 antibody dilution. After 30 minutes incubation at 4° C., cells were washed twice with FACS buffer. Then goat anti-Human IgG (H+L) Secondary Antibody, Alexa Fluor® 647 (Thermo Fisher, cat #A-21445), was added or PE Goat anti-mouse IgG (minimal x-reactivity) Antibody (BioLegend, Cat #405307) was added; the reactions were incubated at 4° C. for 30 minutes (min). The cells were washed twice with wash buffer, and then analyzed for binding by using FACSCalibur (BD Bioscience, San Jose, Calif.) and Flowjo software.

SPR Affinities of Anti-CD47 Antibodies

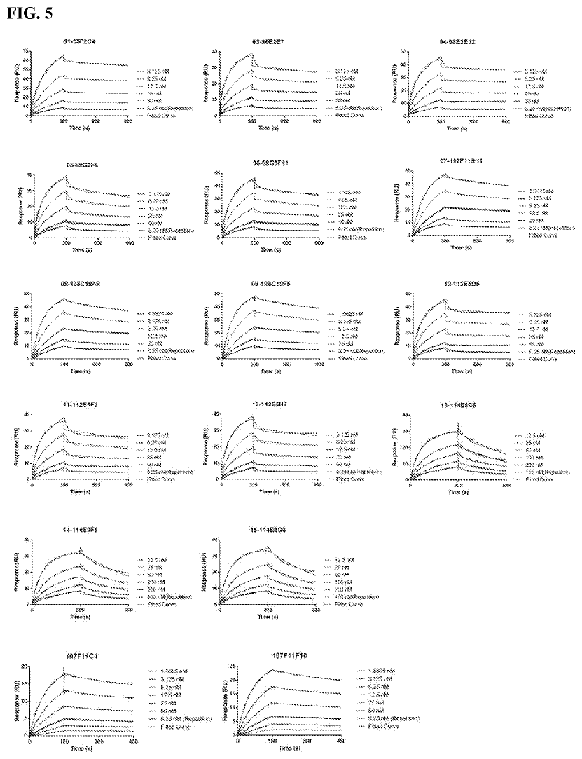

SPR analyses were carried out using a BIACORE™ T200 instrument (GE Healthcare), and kinetic constants were determined using the BIACORE™ T200 evaluation software. Experimental parameters were chosen to ensure that saturation would be reached at the highest antigen concentrations and that Rmax values would be kept under 100 RU. GE anti-Human IgG (Fc-specific, approximately 7,000 RU) was immobilized on a BIACORE™ CM5 chip using EDC-activated amine coupling chemistry. Anti-CD47 antibodies (5 μg/mL, 60 seconds capture time) were then captured using this surface. Next, human CD47-His protein was flowed over captured antibody using a serial dilution series concentration. Captured antibody and antigen were removed between each cycle using 50 mM HCl to ensure a fresh binding surface for each concentration of antigen. The resulting sensorgrams were fit globally using a 1:1 binding model to calculate on- and off-rates (kₐ and k_{d}, respectively), and affinities (K_{D}). The results are shown in FIG. 5.

SIRPα Blocking Activity of CD47 Antibodies

SIRPα is a natural ligand of CD47. The ability of murine antibodies to block the CD47-SIRPα interaction was measured using a flow cytometry based assay. CHO-K1/huCD47, which overexpress human CD47, were incubated with murine CD47 antibodies or a control antibody (B6H12). As shown in FIG. 6, murine antibodies potently blocked the CD47-SIRPα interaction.

In brief, human CD47 transfected CHO cells (GenScript, Cat #M00581) were incubated with human SIRPα (R&D, Cat #4546-SA-050) andante-CD47 antibody dilution. After 30 minutes incubation at 4° C., cells were washed twice with FACS buffer. Then Human SIRP alpha PE-conjugated Antibody (R&D, cat #FAB4546P) was added, incubated at 4° C. for 30 min; cells were washed twice with wash buffer, cells were analyzed for binding by using FACSCalibur (BD Bioscience, San Jose, Calif.) and Flowjo software.

Phagocytosis of Target Cancer Cell Line

CD47 is a cell surface receptor that is upregulated on tumor cells and is also thought to contribute to immune evasion through its interaction with its natural ligand SIPRα. The effect of the murine antibodies on the phagocytosis of target cells were assessed.

Briefly, target cells (PKH26-labeled CCRF/CEM) were treated with anti-CD47 antibody or isotype control for 1 hour, then co-cultured with effector cells (primary human macrophages which were isolated form human peripheral blood and differentiated with M-CSF for 12-14 days in vitro) at a ratio of 1:5 for 1 hour in 37° C./5% CO² incubator. After co-culture, cells were trypsinized and stained with anti-CD11-APC and analyzed by flow cytometry.

Phagocytosis percentage (%) was measured as CD11⁺ PKH26⁺ events as percent of the total PKH26⁺ cells as measured by flow cytometry.

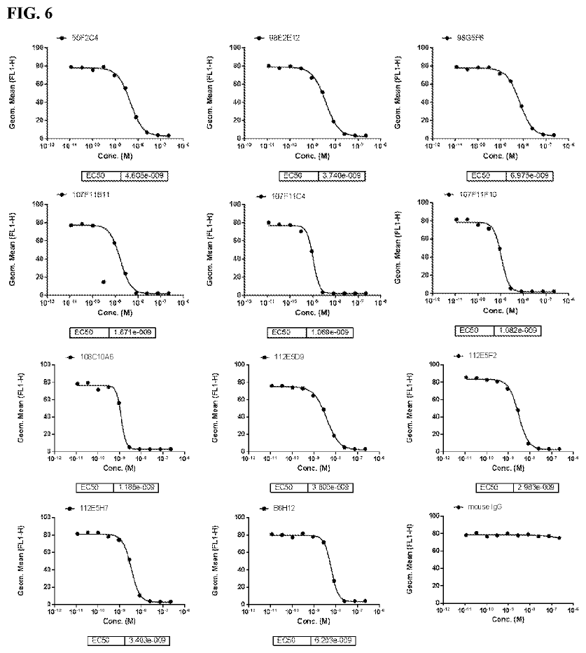

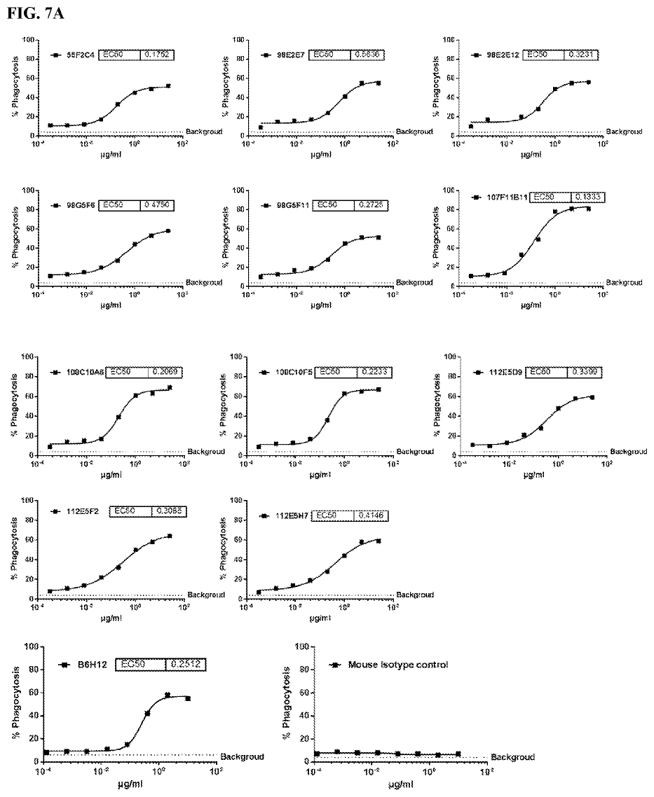

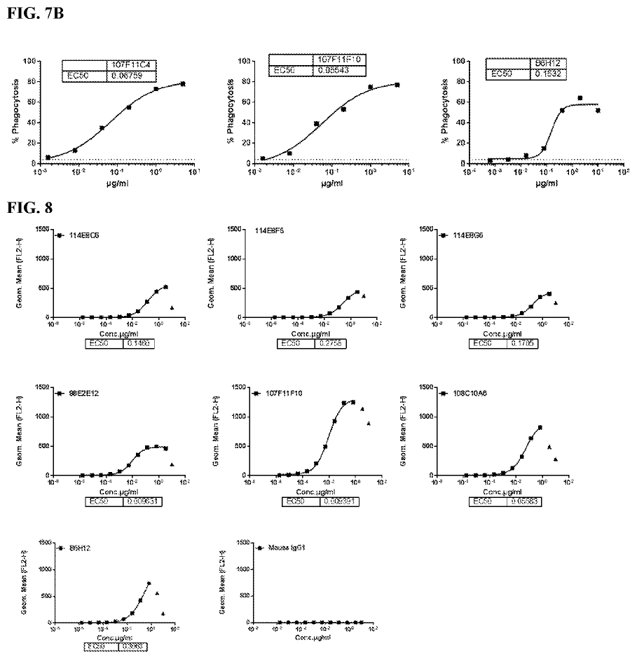

Further, as shown in FIG. 7A and FIG. 7B, these murine CD47 antibodies increased the phagocytosis of the tumor cell line CCRF-CEM.

Phagocytosis of Red Blood Cells

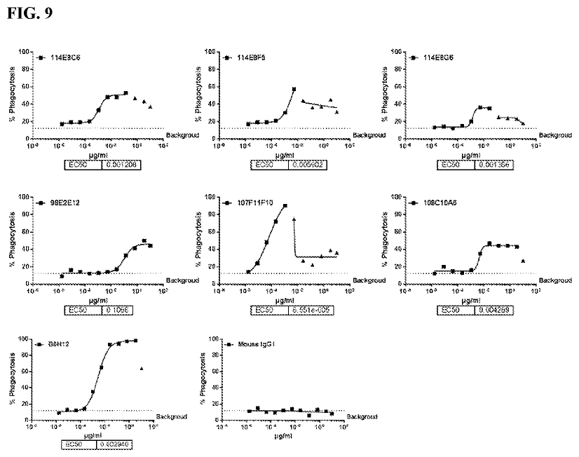

To evaluate the phagocytosis capacity of the murine CD47 antibodies, the ability of murine anti-CD47 antibodies to bind to CD47 on RBC was assessed at first (FIG. 8). All the murine CD47 antibodies bind to RBCs. As shown in FIG. 9, all the murine antibodies increased phagocytic uptake by macrophages.

Briefly, human red blood cells were isolated from healthy donors and labeled with CFSE. Labeled RBCs were pre-incubated with anti-CD47 antibody or isotype control for 1 hour, then co-cultured with human macrophage (isolated form human peripheral blood and differentiated with M-CSF for 12-14 days in vitro) for 1 hour in 37° C./5% CO2 incubator at target-to-effector ratio of 5:1. After co-culture, cells were trypsinized and stained with anti-CD11-APC and analyzed by flow cytometry.

Phagocytosis % was quantitated as CD11⁺ CFSE⁺ events as percent of the total CFSE⁺ cells as measured by flow cytometry.

### Example 3. Sequencing of Anti-CD47 Antibodies

To identify the sequences of the variable regions of the heavy (V_{H}) and light (V_{L}) chains of 13 murine antibodies (55F2C4, 98E2E7, 98E2E12, 98G5F6, 98G5F11, 107F11B11, 107F11C4, 107F11F10, 108C10A6, 108C10F5, 112E5D9, 112E5F2, 112E5H7). Total RNA was isolated from the hybridoma cells and reverse transcribed into cDNA. The variable region of heavy chain and light chain were amplified by mixed specific primers. Sequences were analyzed by IMGT/V-QUEST. The sequences are disclosed in SEQ ID NOs: 349 to 360.

### Example 4. Chimeric Antibody Generation

The DNA of the murine antibodies described above were modified by substituting the coding sequence for human heavy- and light-chain constant domains in place of the homologous murine sequences to generate chimeric antibodies (see U.S. Pat. No. 4,816,567; Morrison, et al., 1984, Proc. Nat. Acad. Sci. USA, 81:6851). The constructs were transfected into mammalian cell lines for chimeric antibodies expression. Secreted antibodies in the condition medium were loaded onto protein A column. After several washes and elute steps, the purified chimeric antibodies were buffer exchanged into 1×PBS buffer. The concentration and purity of the purified chimeric antibodies protein were determined by OD280 and SEC-HPLC, respectively.

Heavy chain sequences of three chimeric antibodies (108C10A6, 98E2E12, and 107F11F10) are disclosed in SEQ ID NOs: 397, 404 and 406. Light chain sequences of three chimeric antibodies (108C10A6, 98E2E12, and 107F11F10) are disclosed in SEQ ID NOs: 398, 405 and 407.

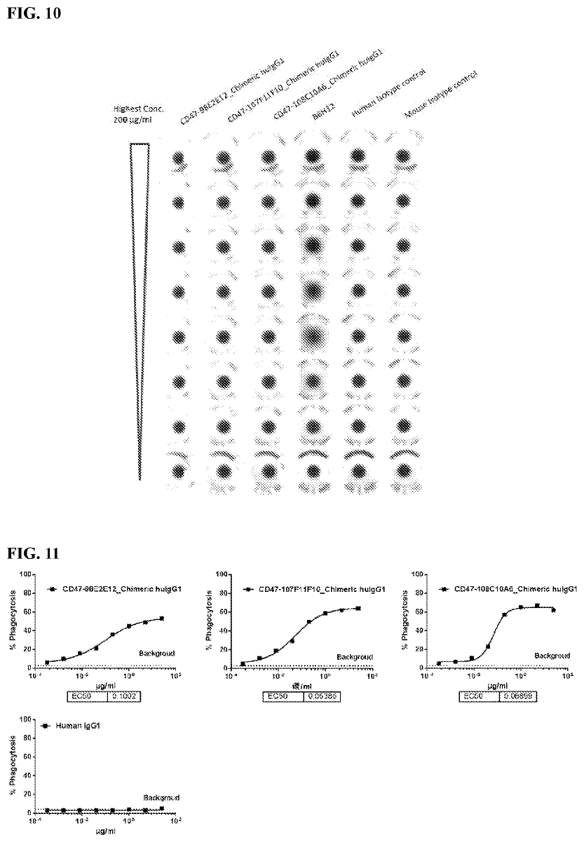

As shown in FIG. 10 and FIG. 11, these chimeric CD47 antibodies increased the phagocytosis of the tumor cell line CCRF-CEM without RBC hemagglutination.

### Example 5. Antibody Humanization

The 108C10A6, 107F11B11 and 98E2E7 antibodies were humanized using CDR grafting technology (see, e.g., U.S. Pat. No. 5,225,539). Briefly, the variable chain sequences of the murine antibody 108C10A6, 107F11B11 and 98E2E7 were compared to those available in the Research Collaboratory for Structural Bioinformatics (RCSB) protein databank. A homology model of 108C10A6, 107F11B11 and 98E2E7 were generated based on the nearest V_{H} and V_{K} structures. Human sequences with highest identity to 108C10A6, 107F11B11 and 98E2E7 were identified and analyzed. Foote and Winter, J. Mol. Biol. 224:487-499 (1992); Morea V. et al., Methods 20:267-279 (2000); Chothia C. et al., J. Mol. Biol. 186:651-663 (1985). The most appropriate human frameworks on which to build the CDR grafted heavy and light chains were identified.

For the heavy chain, the framework encoded by Genbank accession #BAH04539 was determined to be the most appropriate for all three antibodies. For the light chain, the frameworks encoded by Genbank accession #AAC41992, AAZ09096 and ADU32611 were determined to be the most appropriate for 108C10A6, 107F11B11 and 98E2E7, respectively. Straight grafts were performed to generate expression constructs for each chain. The DNA and protein sequences of 108V_{H}1, 108V_{L}1, 107V_{H}1, 107V_{L}1, 98V_{H}1 and 98V_{L}1 are disclosed in the Sequence Listing (SEQ ID NOs: 18, 27, 33, 37, 41 and 46).

In case of affinity loss of the humanized antibodies, several framework residues were mutated back to their murine counterparts to restore the binding affinity of the antibodies. For V_{H} of 108C10A6, humanized variants 108V_{H}2, 108V_{H}3, 108V_{H}4 and 108V_{H}a were made (SEQ ID NOs: 9, 19, 20, 22 and 21). Proline 53 of the CDR2 of 108V_{H}4 was mutated to Alanine to remove the potential acid-labile Asp-Pro hotspot (i.e. 108VH4.M4, SEQ ID NO: 25). For V_{L} of 108C10A6, humanized variants 108VL1.M1, 108VL2.M1 and 108VL3.M1 (SEQ ID NOs: 10, 29, 30, 31) were made. Lysine 24 of the V_{L}-CDR1 was mutated to Arginine in these humanized V_{L} variants.

In case of affinity loss of the humanized antibodies, several framework residues were mutated back to their murine counterparts to restore the binding affinity of the antibodies. For V_{H} of 107F11B11, humanized variants 107VH2 and 107VH3 were made (SEQ ID NOs: 7, 34, 35). For V_{L} of 107F11B11, humanized variants 107VL1.M1 and 107VL2.M1 (SEQ ID NOs: 8, 38, 39) were made. Histidine 24 of the VL-CDR1 was mutated to Arginine in these humanized V_{L} variants.

In case of affinity loss of the humanized antibodies, several framework residues were mutated back to their murine counterparts to restore the binding affinity of the antibodies. For V_{H} of 98E2E7, humanized variants 98V_{H}2, 98V_{H}3 and 98V_{H}a were made (SEQ ID NOs: 3, 42, 43, 44). For V_{L} of 98E2E7, humanized variants 98V_{L}2 and 98V_{L}3 (SEQ ID NOs: 4, 47, 48) were made.

Humanized heavy chains and light chains of the same antibody were mixed and matched to make a series of humanized antibodies. These antibodies were transiently produced using HEK293 cells. Supernatant were collect and subjected to affinity assessment using SPR. Finally, humanized antibody 108VH4.M4_VL1.M1 and 108VH1_VL1.M1 were selected for large-scale production and functional profiling.

Four versions of the humanized antibodies were constructed, namely IgG₄P (with the stabilizing Adair mutation (Angal S. et al., Mol. Immuol. 30:105-108 (1993)), where serine 228 (Kabat numbering) is converted to proline), IgG₄PE (IgG₄P with an addition L235E mutation to further reduce antibody dependent cellular phagocytosis (ADCP) effect), IgG₂ and IgG₁. The heavy chain sequences of IgG₁, IgG₂, IgG₄P and IgG₄PE isotypes of the humanized antibodies are disclosed in SEQ ID NOs: 399, 400, 403 and 401. The light chain sequence is disclosed in SEQ ID NO:402.

### Example 6. In Vitro Characterization of Humanized Anti-CD47 Antibodies

Hemagglutination Activity of Anti-CD47 Antibodies

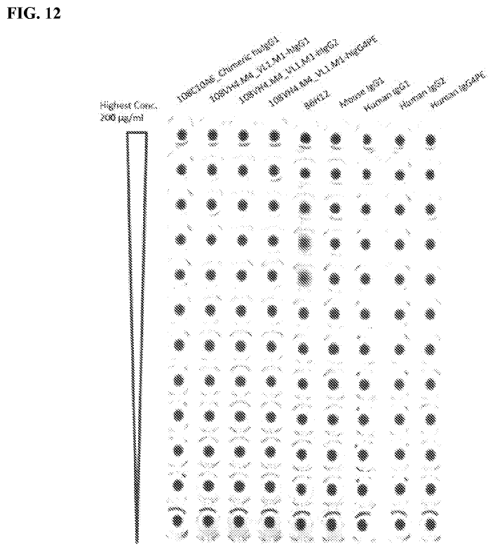

To evaluate whether hemagglutination activity of humanized anti-CD47 antibodies, hemagglutination assay similar to those described in Example 2 above were performed, using human RBCs as targets. As shown in FIG. 12, all the humanized CD47 antibodies did not exhibit hemagglutination activity.

SPR Affinities of Anti-CD47 Antibodies

To measure the binding affinity of humanized anti-CD47 antibodies, SPR assay similar to those described in Example 2 above were performed. As shown in FIG. 13 and table 1, all the humanized CD47 antibodies show K_{D} of 1-2 nM.

Binding to Human CD47

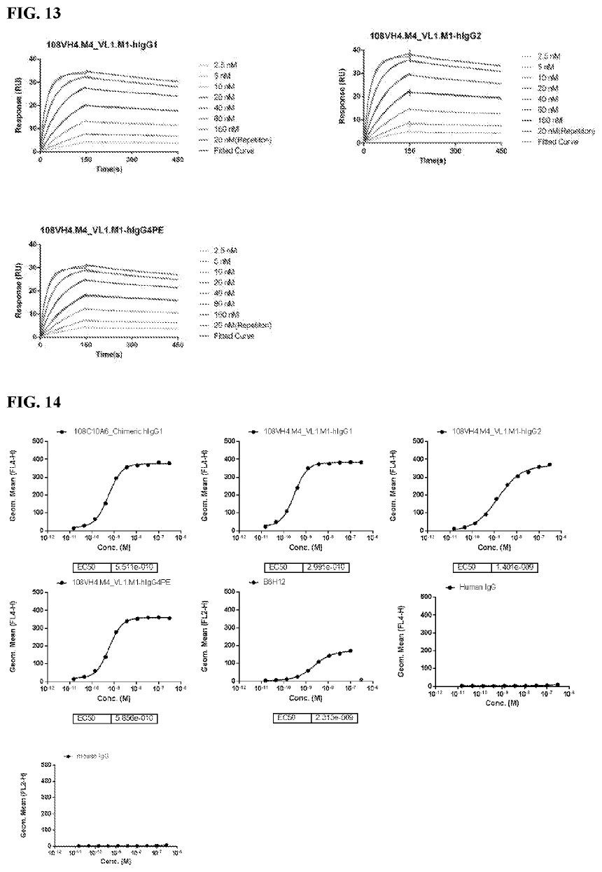

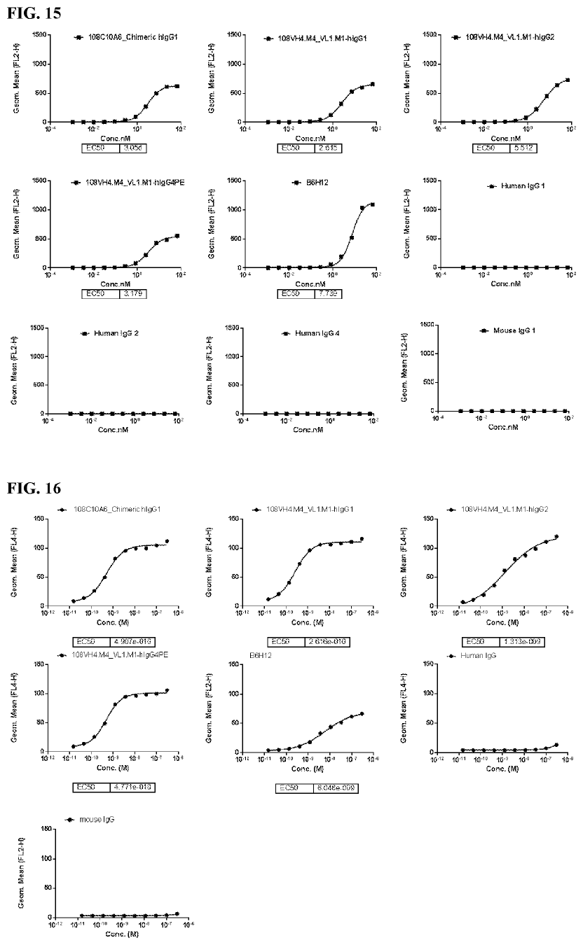

To measure the binding affinity of humanized anti-CD47 antibodies, flow cytometry assay similar to those described in Example 2 above was performed. As shown in FIG. 14 and FIG. 15, all the humanized anti-CD47 antibodies bind to human CD47 over-expressed in CHO-K1 cells (FIG. 14) and RBC (FIG. 15).

Binding to Cynomolgus Monkey CD47

To evaluate the binding of humanized anti-CD47 antibodies to cynomolgus monkey CD47, flow cytometry assay similar to those described in Example 2 above was performed. As shown in FIG. 16, humanized anti-CD47 antibodies binds to cynomolgus monkey CD47.

SIRPα Blocking Activity of Anti-CD47 Antibodies

To evaluate the ability of humanized anti-CD47 antibodies to block the CD47-SIRPα interaction, flow cytometry assay similar to those described in Example 2 above was performed. As shown in FIG. 17, the humanized anti-CD47 antibodies blocked the CD47-SIRPα interaction.

Phagocytosis of Target Cancer Cell Lines

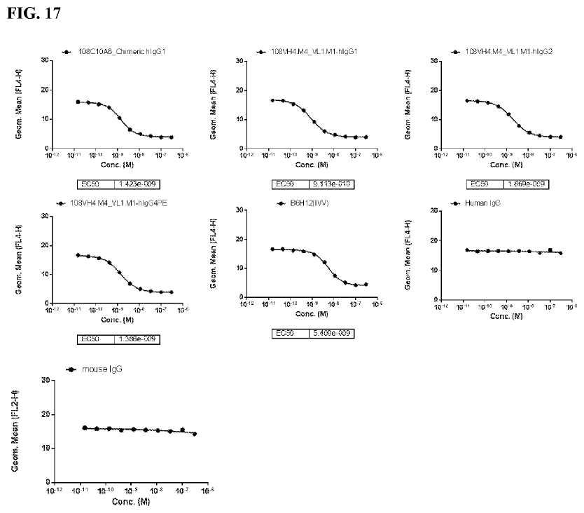

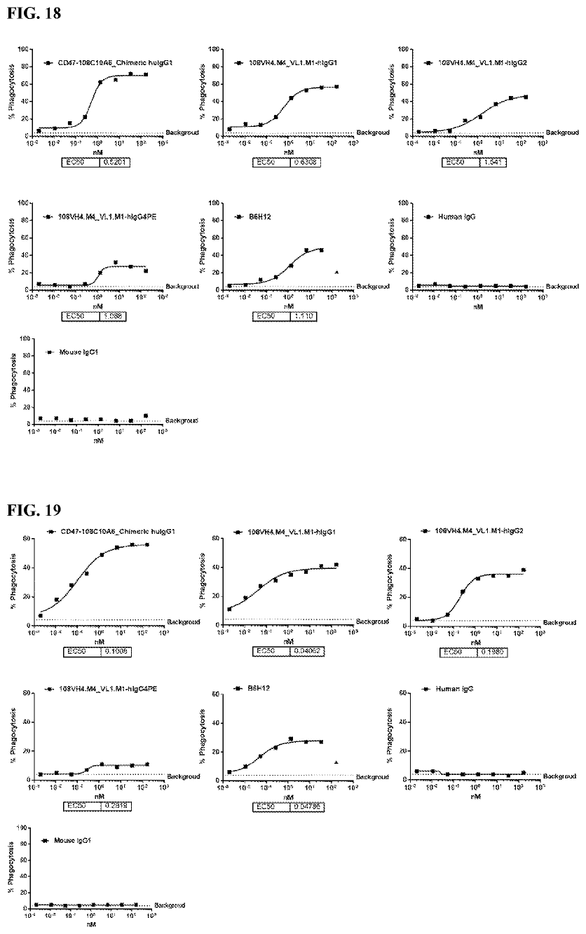

To evaluate the ability of humanized anti-CD47 antibody to enhance the phagocytosis of target cell lines, phagocytosis assay was performed similar to those described in Example 2. As shown in FIG. 18 and FIG. 19, the humanized anti-CD47 antibodies enhanced phagocytosis of CCRF-CEM (FIG. 18) and Raji cells (FIG. 19).

Phagocytosis of Red Blood Cells

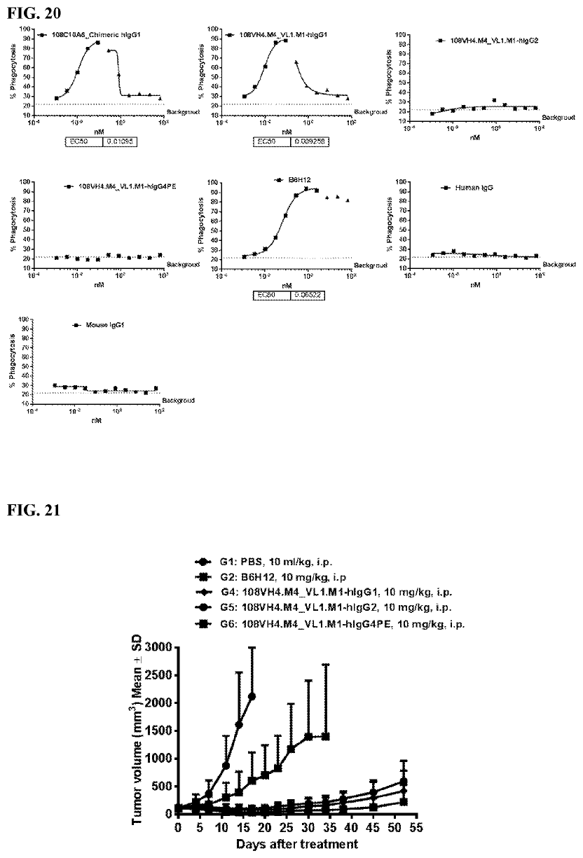

To evaluate whether humanized CD47 antibodies enhance the phagocytosis of RBCs, phagocytosis assay was performed similar to those described in Example 2. As shown in FIG. 20, unexpectedly, IgG₂ and IgG₄PE isotypes of the humanized 108VH4.M4_VL1.M1 did not enhance phagocytosis of RBCs.

### Example 7. Antitumor Activity of Humanized Anti-CD47 Antibodies in Mice Model

The anti-tumor activity of the humanized anti-CD47 antibodies was evaluated in a Raji model of lymphoma. Raji cells were implanted subcutaneously in NOD/SCID mice and randomized into 5 groups (8 mice per group, day 0). Group 1: vehicle (PBS only); Group 2: B6H12 (positive control); Group 3: 108VH4.M4_VL1.M1-hIgG1-Ka; Group 4: 108VH4.M4_VL1.M1-hIgG2-Ka; Group 5: 108VH4.M4_VL1.M1-hIgG4PE-Ka. Treatment with each antibody or vehicle (PBS only) began when tumors were palpable (100 mm³) and mice were euthanized when their tumor volumes reached about 3000 mm³. Tumor volumes were measured 3 times per week. Antibodies were dosed intraperitoneally (i.p.) with 10 mg/kg 3 times per week for 3 weeks (9 total doses per mouse).

As shown in FIG. 21, IgG₁, IgG₂ and IgG₄PE isotypes of the humanized antibodies demonstrated anti-tumor activity in this animal model of lymphoma.

The data indicated that the humanized anti-CD47 antibodies were significantly more potent that the positive antibody B6H12, which was known to bind CD47, block CD47 binding with SIRPα, and suppress tumor growth in mouse models of human cancer.

Anti-tumor activity of these humanized CD47 antibodies were not observed to correlate with their potency of binding CD47, blocking CD47 interaction with SIRPα, or enhancing phagocytosis of tumor cells.

The anti-tumor activity of the humanized anti-CD47 antibody 108VH4.M4_VL1.M1-hIgG₄PE was also evaluated in a SHP-77 model of small cell lung cancer.

As shown in FIG. 23, IgG₄PE isotype of the humanized antibody demonstrated anti-tumor activity in this animal model of small cell lung cancer.

### Example 8. Pharmacokinetics Studies of Humanized CD47 Antibodies in Mice Pharmacokinetic Studies

A mouse PK study was performed with SPF grade female C57BL/6 mice at 7-8 weeks of age (average weight, 20 g). The humanized anti-CD47 antibodies were administered to mice at 3 mg/Kg (volume 10 ml/kg) by i.v. bolus infusion via lateral tail vein. Following i.v. injection, blood samples were taken via the retro-orbital sinus in each group (n=3) at time intervals (15 min, 1 hour (h), 2 h, 4 h, 10 h, 12 h, 24 h, and days 2, 3, 5, 7, 10, 14, 21, 28 and 35 post-injection). Whole blood was immediately taken through capillaries and collected at each time point into microcentrifuge tubes containing heparin. Plasma was extracted and stored at −80° C. until assayed.

Measurement of Anti-CD47 Antibody in Plasma

Anti-CD47 antibody in plasma was measured by enzyme-linked immunosorbent assay (ELISA). Microtiter plates (Costar, Corning) were coated with the capture antibody, goat anti-human IgG Fc fragment specific antibody (Jackson ImmunoResearch), in phosphate buffered saline (PBS). The detection system consisted of peroxidase-conjugated goat anti-human IgG F(ab′)₂ fragment specific antibody (Jackson ImmunoResearch) with tetramethylbenzidine as substrate. Color development was allowed to proceed at room temperature and then terminated with 1 M HCl. The absorbance at 450 nm was determined for all wells using a MK3 microplate reader (ThermoFisher). A standard curve was generated, and samples were quantified by interpolation from the standard curve. Plasma standards were prepared by adding known amounts of anti-CD47 antibody to plasma. These standards were used to calculate the proportion of anti-CD47 antibody recovered by the assay in plasma. A linear regression of anti-CD47 antibody concentration measured by ELISA versus added antibody concentration was performed, and the calculated slope was used as the fractional recovery. The plasma concentrations of anti-CD47 antibody in the samples were then corrected for the recovery.

Pharmacokinetic Analysis

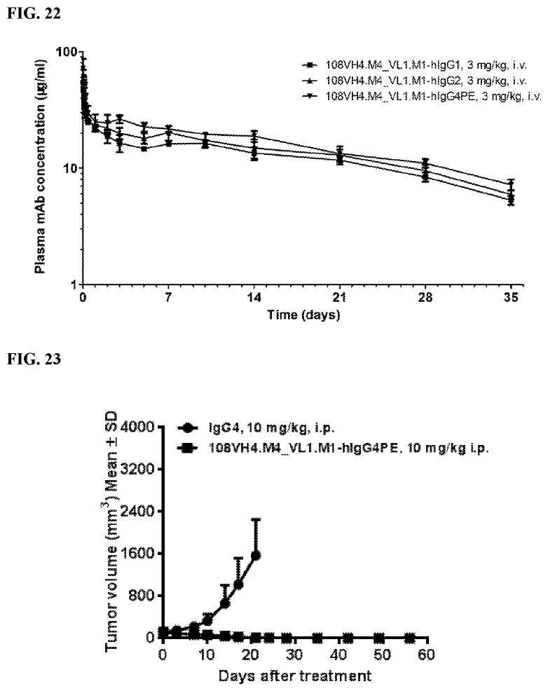

Absorbance units were converted to micrograms per milliliters (μg/ml) of each antibody using the standard curves. Pharmacokinetics parameters were obtained by fitting a non-compartment model to plasma concentration in μg/ml against the time of blood drawing, using WinNonlin Software (WinNonlin ver. 5.2, Pharsight Co.). These include the following parameters: t½, terminal half-life; CL, clearance; Cmax, maximal concentration; Vss, volume of the distribution at a steady state; MRT, mean residence time; AUC, area under the curve. The pharmacokinetics of the tested antibodies were shown in FIG. 22.

TABLE 1
SPR of humanized antibodies

| | | | | | Rmax | Chi² | |
|---|---|---|---|---|---|---|---|
| Ligand | Analyte | kₐ (1/Ms) | k_{d} (1/s) | K_{D} (M) | (RU) | (RU²) | U-value |
| 108C10A6 | | 4.3E+05 | 3.2E−04 | 7.4E−10 | 29.97 | 0.052 | 2 |
| 108VH4.M4_VL1.M1-hlgG1 | human | 3.1E+05 | 4.3E−04 | 1.4E−09 | 34.89 | 0.032 | 1 |
| 108VH4.M4_VL1.M1-hlgG2 | CD47-His | 2.9E+05 | 4.4E−04 | 1.5E−09 | 38.21 | 0.062 | 1 |
| 108VH4.M4_VL1.M1-hlgG4PE | | 3.1E+05 | 4.6E−04 | 1.5E−09 | 31.16 | 0.032 | 1 |

TABLE 2

| | t_{1/2}_Terminal | Cl_pred | Cmax | Vss_pred | AUC_last | AUC_inf |
|---|---|---|---|---|---|---|
| Parameter | (h) | (ml/h/kg) | (μg/ml) | (ml/kg) | (h · μg/ml) | (h · μg/ml) |
| 108VH4.M4_VL1.M1- | 306 ± 43 | 0.23 ± 0.02 | 55 ± 8 | 118 ± 9 | 10,615 ± 702 | 13,041 ± 1,036 |
| hIgG1 | | | | | | |
| 108VH4.M4_VL1.M1- | 335 ± 103 | 0.20 ± 0.03 | 55 ± 14 | 108 ± 15 | 11,934 ± 1,249 | 14,979 ± 1,776 |
| hIgG2 | | | | | | |
| 108VH4.M4_VL1.M1- | 381 ± 81 | 0.17 ± 0.00 | 72 ± 15 | 97 ± 15 | 13,808 ± 934 | 17,919 ± 374 |
| hIgG4PE | | | | | | |

NUCLEIC ACIDS AND AMINO ACID SEQUENCES

| | SEQ | | SEQ | | SEQ | | SEQ | | SEQ | | SEQ | | SEQ | |
|---|---|---|---|---|---|---|---|---|---|---|---|---|---|---|
| | ID | | ID | | ID | | ID | | ID | | ID | | ID | |
| | NO: | FR1 | NO: | CDR1 | NO: | FR2 | NO: | CDR2 | NO: | FR3 | NO: | CDR3 | NO: | FR4 |
| 55F2C4-VH | 1 | QVQLQQSGPQLVR | 49 | GYSFT | 97 | WMKQRPG | 145 | MIDPS | 193 | KATLAVDKSSSTA | 241 | LGRYY | 289 | WGQGT |
| | | PGASVKISCKAS | | NYWMH | | QGLEWIG | | DSETR | | YMQLSSPTSEDSA | | FDY | | TLTVS |
| | | | | | | | | LNQQF | | VYYCAR | | | | S |
| | | | | | | | | KD | | | | | | |
| 55F2C4-VL | 2 | NIVMTQSPKSMYV | 50 | RASEI | 98 | WYQQKPE | 146 | GASNR | 194 | GVPDRFTGSRSAT | 242 | GQSYD | 290 | FGGGT |
| | | SVGERVTLIC | | VGTYV | | QSPKLLI | | YT | | DFSLTISNVQAED | | SPYT | | KLEIK |
| | | | | S | | Y | | | | LADYLC | | | | |
| 98E2E7/ | 3 | QVQLQQSGPQLVR | 51 | GYSFT | 99 | WMKQRPG | 147 | MIDPS | 195 | KATLTVDKSSSTA | 243 | LGRYY | 291 | WGQGT |
| 98E2E12-VH | | PGASVKISCKAS | | NHWMH | | QGLEWIG | | DSETR | | YMQLSSPTSEDSA | | FDY | | TLTVS |
| | | | | | | | | LNWWF | | VFYCAR | | | | S |
| | | | | | | | | KD | | | | | | |
| 98E2E7/ | 4 | NIVMTQSPKSMSV | 52 | RADSI | 100 | WYQQKPE | 148 | GASNR | 196 | GVPDRFTGSRSAT | 244 | GQSYD | 292 | FGGGT |
| 98E2E12-VL | | SVGERVTLSC | | VGTYV | | QSPKLLI | | YT | | DFSLTISNVQAED | | SPYT | | KLEIK |
| | | | | S | | Y | | | | LADYLC | | | | |
| 98G5F6/ | 5 | QVQLQQSGPQLVR | 53 | GYSFT | 101 | WMKQRPG | 149 | MIDPS | 197 | KATLTVDKSSSTA | 245 | LGRYY | 293 | WGQGT |
| 98G5F11-VH | | PGASVKISCKAS | | NYWMH | | QGLEWIG | | DSETR | | YMQLSSPTSEDSA | | FDF | | TLTVS |
| | | | | | | | | LNQQF | | VYYCAR | | | | S |
| | | | | | | | | KD | | | | | | |
| 98G5F6/ | 6 | NIVMTQSPKSMSV | 54 | RASEI | 102 | WYQQKPE | 150 | GASNR | 198 | GVPDRFTGSRSAT | 246 | GQSYD | 294 | FGGGT |
| 98G5F11-VL | | SVGERVTLSC | | VGTYV | | QSPKLLI | | FT | | DFSLTISNVQAED | | SPYT | | KLEIK |
| | | | | S | | Y | | | | LADYLC | | | | |
| 107F11B11/ | 7 | EVQLQQSGAEFVK | 55 | GFNIE | 103 | WVKQRPE | 151 | MIDPA | 199 | KATVTADTSSNTA | 247 | GIGYY | 295 | WGQGT |
| 107F11C4/ | | PGASVKLSCTAS | | DTYMH | | QGLEWIG | | NGKTK | | NLQLSSLTSEDTA | | VGAMD | | SVTVS |
| 107F11F10- | | | | | | | | YGPRF | | VYYCAD | | Y | | S |
| VH | | | | | | | | QD | | | | | | |
| 107F11B11/ | 8 | DIQMNQSPSSLSA | 56 | HASQN | 104 | WYQQKPG | 152 | KASNL | 200 | GVPSRFSGSGSGT | 248 | QQGHS | 296 | FGGGT |
| 107F11C4/ | | SLGDTITITC | | INVWL | | NIPKLLI | | HT | | GFTLTISSLQPED | | YPYT | | KLEIK |
| 107F11F10- | | | | S | | Y | | | | IATYYC | | | | |
| VL | | | | | | | | | | | | | | |
| 108C10A6/ | 9 | QMQLQQSGPQLVR | 57 | GYSFT | 105 | WMKQRPG | 153 | MIDPS | 201 | KATLTVDASSSTA | 249 | LGRYY | 297 | WGQGT |
| 108C10F5- | | PGASVKISCKTS | | HHWIH | | QGLEWIG | | DSETR | | YMQLNSPTSEDSA | | FDY | | TLTVS |
| VH | | | | | | | | LSQKF | | LYFCAR | | | | S |
| | | | | | | | | KD | | | | | | |
| 108C10A6/ | 10 | NIVMTQSPRSMSM | 58 | KASEN | 106 | WYQQKPD | 154 | GASNR | 202 | GVPDRFTGSGSGT | 250 | GESYG | 298 | FGGGT |
| 108C10F5- | | SVGERVTLSC | | VGTYI | | QSPKLLI | | YT | | DFTLTISTVQAED | | HLYT | | KLEIK |
| VL | | | | S | | Y | | | | LADYHC | | | | |
| 112E5D9/ | 11 | QVQLQQSGPQLVR | 59 | GYSFT | 107 | WMKQRPG | 155 | MIDPS | 203 | KATLTVDKTSSTA | 251 | LGRYY | 299 | WGLGT |
| 112E5F2/ | | PGASVKISCKAS | | NNWMH | | QGLEWIG | | DSETR | | YMQLSSPTSEDSA | | FDY | | TLTVS |
| 112E5H7-VH | | | | | | | | LNQQF | | VYYCAR | | | | S |
| | | | | | | | | RD | | | | | | |
| 112E5D9/ | 12 | NIVMTQSPKSMSV | 60 | RASEI | 108 | WYQQKPE | 156 | GAFNR | 204 | GVPDRFTGSRSGT | 252 | GQSYD | 300 | FGGGT |
| 112E5F2/ | | SVGERVTMNC | | VGTYV | | QSPKLLI | | YT | | DFSLNISNVQAED | | SPYT | | KLEIK |
| 112E5H7-VL | | | | S | | Y | | | | LADYLC | | | | |
| 108VH | 13 | QMQLQQSGPQLVR | 61 | GFYST | 109 | WMKQRPG | 157 | MIDPS | 205 | KATLTVDASSSTA | 253 | LGRYY | 301 | WGQGT |
| | | PGASVKISCKTS | | HHWIH | | QGLEWIG | | DSETR | | YMQLNSPTSEDSA | | FDY | | TLTVS |
| | | | | | | | | LSQKF | | LYFCAR | | | | S |
| | | | | | | | | KD | | | | | | |
| 108VH.M1 | 14 | QMQLQQSGPQLVR | 62 | GYSFT | 110 | WMKQRPG | 158 | IIDPS | 206 | KATLTVDASSSTA | 254 | LGRYY | 302 | WGQGT |
| | | PGASVKISCKTS | | HHWIH | | QGLEWIG | | DSETR | | YMQLNSPTSEDSA | | FDY | | TLTVS |
| | | | | | | | | LSQKF | | LYFCAR | | | | S |
| | | | | | | | | KD | | | | | | |
| 108VH.M2 | 15 | QMQLQQSGPQLVR | 63 | GYSFT | 111 | WMKQRPG | 159 | MIEPS | 207 | KATLTVDASSSTA | 255 | LGRYY | 303 | WGQGT |
| | | PGASVKISCKTS | | HHWIH | | QGLEWIG | | DSETR | | YMQLNSPTSEDSA | | FDY | | TLTVS |
| | | | | | | | | LSQKF | | LYFCAR | | | | S |
| | | | | | | | | KD | | | | | | |
| 108VH.M3 | 16 | QMQLQQSGPQLVR | 64 | GYSFT | 112 | WMKQRPG | 160 | MISPS | 208 | KATLTVDASSSTA | 256 | LGRYY | 304 | WGQGT |
| | | PGASVKISCKTS | | HHWIH | | QGLEWIG | | DSETR | | YMQLNSPTSEDSA | | FDY | | TLTVS |
| | | | | | | | | LSQKF | | LYFCAR | | | | S |
| | | | | | | | | KD | | | | | | |
| 108VH.M4 | 17 | QMQLQQSGPQLVR | 65 | GYSFT | 113 | WMKQRPG | 161 | MIDAS | 209 | KATLTVDASSSTA | 257 | LGRYY | 305 | WGQGT |
| | | PGASVKISCKTS | | HHWIH | | QGLEWIG | | DSETR | | YMQLNSPTSEDSA | | FDY | | TLTVS |
| | | | | | | | | LSQKF | | LYFCAR | | | | S |
| | | | | | | | | KD | | | | | | |
| 108VH1 | 18 | QVQLVQSGAEVKK | 66 | GYSFT | 114 | WVRQAPG | 162 | MIDAS | 210 | RVTITADKSTSTA | 258 | LGRYY | 306 | WGQGT |
| | | PGSSVKVSCKAS | | HHWIH | | QGLEWMG | | DSETR | | YMELSSLRSEDTA | | FDY | | TLTVS |
| | | | | | | | | LSQKF | | VYYCAR | | | | S |
| | | | | | | | | KD | | | | | | |
| 108VH2 | 19 | EVQLVQSGAEVKK | 67 | GYSFT | 115 | WMRQAPG | 163 | MIDAS | 211 | RVTITADKSTSTA | 259 | LGRYY | 307 | WGQGT |
| | | PGSSVKVSCKAS | | HHWIH | | QGLEWIG | | DSETR | | YMELSSLRSEDTA | | FDY | | TLTVS |
| | | | | | | | | LSQKF | | VYYCAR | | | | S |
| | | | | | | | | KD | | | | | | |
| 108VH3 | 20 | EVQLVQSGAEVKK | 68 | GYSFT | 116 | WMRQAPG | 164 | MIDAS | 212 | RATLTVDKSTSTA | 260 | LGRYY | 308 | WGQGT |
| | | PGSSVKVSCKAS | | HHWIH | | QGLEWIG | | DSETR | | YMELSSLRSEDTA | | FDY | | TLTVS |
| | | | | | | | | LSQKF | | VYFCAR | | | | S |
| | | | | | | | | KD | | | | | | |
| 108Vha | 21 | EVQLVQSGAEVKK | 69 | GYSFT | 117 | WMKQAPG | 165 | MIDPS | 213 | KATLTVDKSTSTA | 261 | LGRYY | 309 | WGQGT |
| | | PGSSVKVSCKTS | | HHWIH | | QGLEWIG | | DSETR | | YMELSSLRSEDTA | | FDY | | TVTVS |
| | | | | | | | | LSQKF | | VYFCAR | | | | S |
| | | | | | | | | KD | | | | | | |
| 108VH4 | 22 | EVQLVQSGAEVKK | 70 | GYSFT | 118 | WVRQAPG | 166 | MIDPS | 214 | RVTITADKSTSTA | 262 | LGRYY | 310 | WGQGT |
| | | PGSSVKVSCKAS | | HHWIH | | QGLEWMG | | DSETR | | YMELSSLRSEDTA | | FDY | | TVTVS |
| | | | | | | | | LSQKF | | VYYCAR | | | | S |
| | | | | | | | | KD | | | | | | |
| 108VH4.M2 | 23 | EVQLVQSGAEVKK | 71 | GYSFT | 119 | WVRQAPG | 167 | MIEPS | 215 | RVTITADKSTSTA | 263 | LGRYY | 311 | WGQGT |
| | | PGSSVKVSCKAS | | HHWIH | | QGLEWMG | | DSETR | | YMELSSLRSEDTA | | FDY | | TVTVS |
| | | | | | | | | LSQKF | | VYYCAR | | | | S |
| | | | | | | | | KD | | | | | | |
| 108VH4.M3 | 24 | EVQLVQSGAEVKK | 72 | GYSFT | 120 | WVRQAPG | 168 | MISPS | 216 | RVTITADKSTSTA | 264 | LGRYY | 312 | WGQGT |
| | | PGSSVKVSCKAS | | HHWIH | | QGLEWMG | | DSETR | | YMELSSLRSEDTA | | FDY | | TVTVS |
| | | | | | | | | LSQKF | | VYYCAR | | | | S |
| | | | | | | | | KD | | | | | | |
| 108VH4.M4 | 25 | EVQLVQSGAEVKK | 73 | GYSFT | 121 | WVRQAPG | 169 | MIDAS | 217 | RVTITADKSTSTA | 265 | LGRYY | 313 | WGQGT |
| | | PGSSVKVSCKAS | | HHWIH | | QGLEWMG | | DSETR | | YMELSSLRSEDTA | | FDY | | TVTVS |
| | | | | | | | | LSQKF | | VYYCAR | | | | S |
| | | | | | | | | KD | | | | | | |
| 108VL | 26 | NIVMTQSPRSMSM | 74 | KASEN | 122 | WYQQKPD | 170 | GASNR | 218 | GVPDRFTGSGSGT | 266 | GESYG | 314 | FGGGT |
| | | SVGERVTLSC | | VGTYI | | QSPKLLI | | YT | | DFTLTISTVQAED | | HLYT | | KLEIK |
| | | | | S | | Y | | | | LADYHC | | | | |
| 108VL1 | 27 | EIVLTQSPATLSL | 75 | KASEN | 123 | WYQQKPG | 171 | GASNR | 219 | GIPARFSGSGSGT | 267 | GESYG | 315 | FGGGT |
| | | SPGERATLSC | | VGTYI | | QAPRLLI | | YT | | DFTLTISSLEPED | | HLYT | | KLEIK |
| | | | | S | | Y | | | | FAVYYC | | | | |
| 108V.M1 | 28 | EIVLTQSPATLSL | 76 | KASEN | 124 | WYQQKPG | 172 | GASNR | 220 | GIPARFSGSGSGT | 268 | GESYG | 316 | FGGGT |
| | | SPGERATLSC | | VGTYI | | QAPRLLI | | YT | | DFTLTISSLEPED | | HLYT | | KLEIK |
| | | | | S | | Y | | | | FAVYYC | | | | |
| 108VL1.M1 | 29 | EIVLTQSPATLSL | 77 | RASEN | 125 | WYQQKPG | 173 | GASNR | 221 | GIPARFSGSGSGT | 269 | GESYG | 317 | FGGGT |
| | | SPGERATLSC | | VGTYI | | QAPRLLI | | YT | | DFTLTISSLEPED | | HLYT | | KVEIK |
| | | | | S | | Y | | | | FAVYYC | | | | |
| 108VL2.M1 | 30 | EIVLTQSPATLSL | 78 | RASEN | 126 | WYQQKPG | 174 | GASNR | 222 | GVPARFSGSGSGT | 270 | GESYG | 318 | FGGGT |
| | | SPGERATLSC | | VGTYI | | QAPRLLI | | YT | | DFTLTISSLEPED | | HLYT | | KVEIK |
| | | | | S | | Y | | | | FAVYHC | | | | |
| 108VL3.M1 | 31 | EIVLTQSPATLSL | 79 | RASEN | 127 | WYQQKPG | 175 | GASNR | 223 | GVPARFSGSGSGT | 271 | GESYG | 319 | FGGGT |
| | | SPGERATLSC | | VGTYI | | QAPRLLI | | YT | | DFTLTISSLEPED | | HLYT | | KVEIK |
| | | | | S | | Y | | | | FAVYHC | | | | |
| 107VH | 32 | EVQLQQSGAEFVK | 80 | GFNIE | 128 | WVKQRPE | 176 | MIDPA | 224 | KATVTADTSSNTA | 272 | GIGYY | 320 | WGQGT |
| | | PGASVKLSCTAS | | DTYMH | | QGLEWIG | | NGKTK | | NLQLSSLTSEDTA | | VGAMD | | SVTVS |
| | | | | | | | | YGPRF | | VYYCAD | | Y | | S |
| | | | | | | | | QD | | | | | | |
| 17VH1 | 33 | EVQLVQSGAEVKK | 81 | GFNIE | 129 | WVRQAPG | 177 | MIDPA | 225 | RVTITADKSTSTA | 273 | GIGYY | 321 | WGQGT |
| | | PGSSVKVSCKAS | | DTYMH | | QGLEWMG | | NGKTK | | YMELSSLRSEDTA | | VGAMD | | SVTVS |
| | | | | | | | | YGPRF | | VYYCAR | | Y | | S |
| | | | | | | | | QD | | | | | | |
| 107VH2 | 34 | EVQLVQSGAEVKK | 82 | GFNIE | 130 | WVRQAPG | 178 | MIDPA | 226 | RVTITADKSTSTA | 274 | GIGYY | 322 | WGQGT |
| | | PGSSVKVSCKAS | | DTYMH | | QGLEWIG | | NGKTK | | YMELSSLRSEDTA | | VGAMD | | SVTVS |
| | | | | | | | | YGPRF | | VYYCAR | | Y | | S |
| | | | | | | | | QD | | | | | | |
| 107VH3 | 35 | EVQLVQSGAEVKK | 83 | GFNIE | 131 | WVRQAPG | 179 | MIDPA | 227 | RVTITADKSTSTA | 275 | GIGYY | 323 | WGQGT |
| | | PGSSVKVSCKAS | | DTYMH | | QGLEWIG | | NGKTK | | YMELSSLRSEDTA | | VGAMD | | SVTVS |
| | | | | | | | | YGPRF | | VYYCAR | | Y | | S |
| | | | | | | | | QD | | | | | | |
| 107VL | 36 | DIQMNQSPSSLSA | 84 | HASQN | 132 | WYQQKPG | 180 | KASNL | 228 | GVPSRFSGSGSGT | 276 | QQGHS | 324 | FGGGT |
| | | SLGDTITITC | | INVWL | | NIPKLLI | | HT | | GFTLTISSLQPED | | YPYT | | KLEIK |
| | | | | S | | Y | | | | IATYYC | | | | |
| 107VL1 | 37 | DIQMTQSPSSLSA | 85 | HASQN | 133 | WYQQKPG | 181 | KASNL | 229 | GVPSRFSGSGSGT | 277 | QQGHS | 325 | FGGGT |
| | | SVGDRVTITC | | INVWL | | KAPKLLI | | HT | | GFTLTISSLQPED | | YPYT | | KVEIK |
| | | | | S | | Y | | | | IATYYC | | | | |
| 107VL1-M1 | 38 | DIQMTQSPSSLSA | 86 | RASQN | 134 | WYQQKPG | 182 | KASNL | 230 | GVPSRFSGSGSGT | 278 | QQGHS | 326 | FGGGT |
| | | SVGDRVTITC | | INVWL | | KAPKLLI | | HT | | DFTLTISSLQPED | | YPYT | | KVEIK |
| | | | | S | | Y | | | | FATYYC | | | | |
| 107VL2.M1 | 39 | DIQMTQSPSSLSA | 87 | RASQN | 135 | WYQQKPG | 183 | KASNL | 231 | GVPSRFSGSGSGT | 279 | QQGHS | 327 | FGGGT |
| | | SVGDRVTITC | | INVWL | | KAPKLLI | | HT | | DFTLTISSLQPED | | YPYT | | KLEIK |
| | | | | S | | Y | | | | FATYYC | | | | |
| 98VH | 40 | QVQLQQSGPQLVR | 88 | GYSFT | 136 | WMKQRPG | 184 | MIDPS | 232 | KATLTVDKSSSTA | 280 | LGRYY | 328 | WGQGT |
| | | PGASVKISCKAS | | NHWMH | | QGLEWIG | | DSETR | | YMQLSSPTSEDSA | | FDY | | TLTVS |
| | | | | | | | | LNQQF | | VFYCAR | | | | S |
| | | | | | | | | KD | | | | | | |
| 98VH1 | 41 | QVQLQQSGPQLVR | 89 | GYSFT | 137 | WVRQAPG | 185 | MIDPS | 233 | KATLTVDKSSSTA | 281 | LGRYY | 329 | WGQGT |
| | | PGASVKISCKAS | | NHWMH | | QGLEWMG | | DSETR | | YMQLSSPTSEDSA | | FDY | | TLTVS |
| | | | | | | | | LNQQF | | VFYCAR | | | | S |
| | | | | | | | | KD | | | | | | |
| 98VH2 | 42 | QVQLQQSGPQLVR | 90 | GYSFT | 138 | WMRQAPG | 186 | MIDPS | 234 | KATLTVDKSSSTA | 282 | LGRYY | 330 | WGQGT |
| | | PGASVKISCKAS | | NHWMH | | QGLEWIG | | DSETR | | YMQLSSPTSEDSA | | FDY | | TLTVS |
| | | | | | | | | LNQQF | | VFYCAR | | | | S |
| | | | | | | | | KD | | | | | | |
| 98VH3 | 43 | QVQLQQSGPQLVR | 91 | GYSFT | 139 | WMRQAPG | 187 | MIDPS | 235 | KATLTVDKSSSTA | 283 | LGRYY | 331 | WGQGT |
| | | PGASVKISCKAS | | NHWMH | | QGLEWIG | | DSETR | | YMQLSSPTSEDSA | | FDY | | TLTVS |
| | | | | | | | | LNQQF | | VFYCAR | | | | S |
| | | | | | | | | KD | | | | | | |
| 98VHa | 44 | QVQLQQSGPQLVR | 92 | GYSFT | 140 | WMKQAPG | 188 | MIDPS | 236 | KATLTVDKSSSTA | 284 | LGRYY | 332 | WGQGT |
| | | PGASVKISCKAS | | NHWMH | | QGLEWIG | | DSETR | | YMQLSSPTSEDSA | | FDY | | TLTVS |
| | | | | | | | | LNQQF | | VFYCAR | | | | S |
| | | | | | | | | KD | | | | | | |
| 98VL | 45 | NIVMTQSPKSMSV | 93 | RASDI | 141 | WYQQKPE | 189 | GASNR | 237 | GVPDRFTGSRSAT | 285 | GQSYD | 333 | FGGGT |
| | | SVGERVTLSC | | VGTYV | | QSPKLLI | | YT | | DFSLTISNVQAED | | SPYT | | KLEIK |
| | | | | S | | Y | | | | LADYLC | | | | |
| 98VL1 | 46 | EIVMTQSPATLSV | 94 | RASDI | 142 | WYQQKPG | 190 | GASNR | 238 | GIPARFSGSGSGT | 286 | GQSYD | 334 | FGGGT |
| | | SPGERATLSC | | VGTYV | | QAPRLLI | | YT | | EFTLTISSLQSED | | SPYT | | KVEIK |
| | | | | S | | Y | | | | FAVYYC | | | | |
| 98VL2 | 47 | EIVMTQSPATLSV | 95 | RASDI | 143 | WYQQKPG | 191 | GASNR | 239 | GIPARFSGSGSGT | 287 | GQSYD | 335 | FGGGT |
| | | SPGERATLSC | | VGTYV | | QAPRLLI | | YT | | EFTLTISSLQSED | | SPYT | | KVEIK |
| | | | | S | | Y | | | | FAVYYC | | | | |
| 98VL3 | 48 | EIVMTQSPATLSV | 96 | RASDI | 144 | WYQQKPG | 192 | GASNR | 240 | GVPARFSGSGSGT | 288 | GQSYD | 336 | FGGGT |
| | | SPGERATLSC | | VGTYV | | QAPRLLI | | YT | | EFTLTISSVQSED | | SPYT | | KLEIK |
| | | | | S | | Y | | | | FAVYLC | | | | |

Nucleic acid sequences of exemplary disclosed antibodies (V_{H}/V_{L})

| | |
|---|---|
| SEQ ID NO.: 337- | CAGGTGCAACTGCAGCAGTCTGGGCCTCAGCTGGTTAGGCCTGGGGCTTCAGTGAAGATATC |
| 55F2C4-VH | CTGCAAGGCTTCTGGTTACTCATTCACCAACTACTGGATGCACTGGATGAAGCAGAGGCCTG |
| | GACAAGGTCTTGAATGGATTGGCATGATTGATCCTTCCGATAGTGAGACTAGGTTAAATCAG |
| | CAGTTCAAGGACAAGGCCACATTGGCTGTTGACAAATCCTCCAGCACAGCCTACATGCAACT |
| | CAGCAGCCCGACATCTGAGGACTCTGCGGTCTATTACTGTGCAAGATTAGGGCGGTATTATT |
| | TTGACTACTGGGGCCAAGGCACCACTCTCACAGTCTCCTCA |
| SEQ ID NO.: 338- | AACATTGTAATGACCCAATCTCCCAAATCCATGTACGTGTCAGTCGGGGAGAGGGTCACCTT |
| 55F2C4-VL | GATCTGCAGGGCCAGTGAGATTGTGGGCACTTATGTTTCCTGGTATCAACAGAAACCAGAGC |
| | AGTCTCCTAAATTGCTGATATACGGGGCATCCAACCGGTACACTGGGGTCCCCGATCGCTTC |
| | ACAGGCAGTAGATCTGCAACAGATTTCAGTCTGACCATCAGTAATGTGCAGGCTGAAGACCT |
| | TGCAGATTATCTCTGTGGACAGAGTTACGACTCTCCGTACACGTTCGGAGGGGGGACCAAGC |
| | TGGAAATAAAA |
| SEQ ID NO: 339- | CAGGTGCAACTGCAGCAGTCTGGGCCTCAGCTAGTTAGGCCTGGGGCTTCAGTGAAGATATC |
| 98E2E7/98E2E7-VH | CTGCAAGGCTTCTGGTTACTCATTCACCAACCACTGGATGCACTGGATGAAGCAGAGGCCTG |
| | GACAAGGTCTTGAATGGATTGGCATGATTGATCCTTCCGATAGTGAGACTAGGTTAAATCAG |
| | CAGTTCAAGGACAAGGCCACATTGACTGTTGACAAATCCTCCAGCACAGCCTACATGCAACT |
| | CAGCAGCCCGACATCTGAGGACTCTGCGGTCTTTTACTGTGCAAGATTAGGGCGGTATTATT |
| | TTGACTACTGGGGCCAAGGCACCACTCTCACAGTCTCCTCA |
| SEQ ID NO: 340- | AACATTGTAATGACCCAATCTCCCAAATCCATGTCCGTGTCAGTCGGGGAGAGGGTCACCTT |
| 98E2E7/98E2E7-VL | GAGCTGCAGGGCCAGTGACATTGTGGGCACTTATGTTTCCTGGTATCAACAGAAACCAGAGC |
| | AGTCTCCTAAATTGCTGATATATGGGGCATCCAACCGGTACACTGGGGTCCCCGATCGCTTC |
| | ACAGGCAGTAGATCTGCAACAGATTTCAGTCTGACCATCAGTAATGTGCAGGCTGAAGACCT |
| | TGCAGATTATCTCTGTGGACAGAGTTACGACTCTCCGTACACGTTCGGAGGGGGGACCAAGC |
| | TGGAAATAAAA |
| SEQ ID NO: 341- | CAGGTGCAACTGCAGCAGTCTGGGCCTCAGCTAGTTAGGCCTGGGGCTTGCAGTGAAGATAT |
| 98G5F5/98G5F11-VH | CCTGCAAGGCTTCTGGTTACTCATTCACCAACTACTGGATGCACTGGATGAAGCAGAGGCCT |
| | GGACAAGGTCTTGAATGGATTGGCATGATTGATCCTTCCGATAGTGAGACTAGGTTAAATCA |
| | GCAGTTCAAGGACAAGGCCACATTGACTGTTGACAAATCCTCCAGCACAGCCTACATGCAAC |
| | TCAGCAGCCCGACATCTGAGGACTCTGCGGTCTATTATTGTGCAAGATTAGGGAGGTATTAT |
| | TTTGACTTCTGGGGCCAAGGCACCACTCTCACAGTCTCCTCA |
| SEQ ID NO: 342- | AACATTGTAATGACCCAATCTCCCAAATCCATGTCCGTGTCAGTCGGGGAGAGGGTCACCTT |
| 98G5F6/98G5F11-VL | GAGCTGCAGGGCCAGTGAGATTGTGGGCACTTATGTTTCCTGGTATCAACAGAAACCAGAGC |
| | AGTCTCCTAAATTGCTGATATATGGGGCATCCAACCGGTTCACTGGGGTCCCCGATCGCTTC |
| | ACAGGCAGTAGATCTGCAACAGATTTCAGTCTGACCATCAGTAATGTGCAGGCTGAAGACCT |
| | TGCAGATTATCTCTGTGGACAGAGTTACGACTCTCCGTACACGTTCGGAGGGGGGACCAAGC |
| | TGGAAATAAAG |
| SEQ ID NO: 343- | CAGATGCAACTGCAGCAGTCTGGGCCTCAACTGGTTAGGCCTGGGGCTTCAGTGAAGATATC |
| 108C10A6/108C10F5-VH | CTGCAAGACTTCTGGTTACTCATTCACCCACCACTGGATACACTGGATGAAGCAGAGGCCTG |
| | GACAAGGTCTTGAGTGGATTGGCATGATTGATCCCTCCGATAGTGAAACTAGATTAAGTCAG |
| | AAGTTCAAGGACAAGGCCACATTGACTGTAGACGCATCCTCCAGCACAGCCTACATGCAACT |
| | CAACAGCCCGACATCTGAAGACTCTGCGCTCTATTTCTGTGCAAGATTAGGGCGGTACTACT |
| | TTGACTACTGGGGCCAAGGAACCACTCTCACAGTCTCCTCA |
| SEQ ID NO: 344- | AACATTGTCATGACCCAGTCTCCCAGATCCATGTCCATGTCAGTTGGAGAGAGGGTCACCTT |
| 108C10A6/108C10F5-VL | GAGCTGCAAGGCCAGTGAGAATGTGGGTACTTATATATCCTGGTATCAACAGAAACCAGACC |
| | AGTCTCCTAAACTGCTGATATACGGGGCATCCAACCGGTACACTGGGGTCCCCGATCGCTTC |
| | ACAGGCAGTGGATCTGGAACAGATTTCACTCTGACCATCAGCACTGTGCAGGCTGAAGACCT |
| | TGCAGATTATCACTGTGGAGAGAGTTATGGTCATCTGTACACGTTCGGAGGGGGGACCAAGC |
| | TGGAAATAAAA |
| SEQ ID NO: 345- | CAGGTGCAACTGCAGCAGTCTGGGCCTCAACTGGTTAGGCCTGGGGCTTCAGTGAAGATTTC |
| 112E5D9/112E5F2/112E7H7-VH | CTGCAAGGCTTCTGGTTACTCTTTCACCAATAACTGGATGCACTGGATGAAACAGAGGCCTG |
| | GACAAGGTCTTGAATGGATTGGCATGATTGATCCTTCCGATAGTGAGACCAGGTTAAATCAG |
| | CAGTTCAGGGACAAGGCCACATTGACTGTTGACAAAACCTCCAGCACAGCCTACATGCAACT |
| | CAGCAGCCCGACATCTGAGGACTCTGCGGTCTATTACTGTGCAAGATTAGGGCGGTATTATT |
| | TTGACTACTGGGGCCTAGGCACCACTCTCACAGTCTCCTCA |
| SEQ ID NO: 346- | AATATTGTAATGACCCAATCTCCCAAATCCATGTCCGTGTCAGTCGGGGAGAGGGTCACAAT |
| 112E5D9/112E5F2/112E5H7-VL | GAACTGCAGGGCCAGTGAGATTGTGGGCACTTATGTTTCCTGGTATCAACAAAAACCAGAGC |
| | AGTCTCCTAAATTGCTAATATACGGGGCATTCAACCGCTACACTGGGGTCCCCGATCGCTTC |
| | ACTGGCAGTAGATCTGGAACAGATTTCAGTCTGAACATCAGTAATGTGCAGGCTGAAGACCT |
| | TGCAGATTATCTCTGTGGACAGAGTTACGACTCTCCGTACACGTTCGGAGGGGGGACCAAGC |
| | TGGAAATAAAA |
| SEQ ID NO: 347- | GAGGTTCAGCTGCAGCAGTCTGGGGCAGAGTTTGTGAAGCCAGGGGCCTCAGTCAAGTTGTC |
| 107F11B11/107F11C4/ | CTGCACAGCTTCTGGCTTCAATATTGAAGACACCTATATGCACTGGGTGAAGCAGAGGCCTG |
| 107F11F10-VH | AACAGGGCCTGGAGTGGATTGGAATGATTGATCCTGCGAATGGTAAAACTAAATATGGCCCG |
| | AGGTTCCAGGACAAGGCCACTGTAACAGCAGACACATCCTCCAACACAGCCAACCTGCAGCT |
| | CAGCAGCCTGACATCTGAGGACACTGCCGTCTATTACTGTGCTGACGGAATTGGTTACTACG |
| | TAGGGGCTATGGACTACTGGGGTCAAGGAACCTCAGTCACCGTCTCCTCA |
| SEQ ID NO: 348- | GACATCCAGATGAACCAGTCTCCATCCAGTCTGTCTGCATCCCTTGGAGACACAATTACCAT |
| 107F11B11/107F11C4/ | CACTTGCCATGCCAGTCAGAACATTAATGTTTGGTTAAGCTGGTACCAGCAGAAACCAGGAA |
| 107F11F10-VL | ATATTCCTAAACTATTGATCTATAAGGCTTCCAACTTGCACACAGGCGTCCCATCAAGGTTT |
| | AGTGGCAGTGGATCTGGAACAGGTTTCACATTAACCATCAGCAGCCTGCAGCCTGAAGACAT |
| | TGCCACTTACTACTGTCAACAGGGTCACAGTTATCCGTACACGTTCGGAGGGGGGACCAAGC |
| | TGGAAATAAAA |

amino acid sequences of exemplary disclosed antibody (VH/VL);
CDRs are underlined; HUMAN CD47

| | |
|---|---|
| SEQ ID NO: 349- | QVQLQQSGPQLVRPGASVKISCKASGYSFTNYWMHWMKQRPGQGLEWIGMIDPSDSETRLNQ |
| 55F2C4-VH | QFKDKATLAVDKSSSTAYMQLSSPTSEDSAVYYCARLGRYYFDYWGQGTTLTVSS |
| SEQ ID NO: 350- | NIVMTQSPKSMYVSVGERVTLICRASEIVGTYVSWYQQKPEQSPKLLIYGASNRYTGVPDRF |
| 55F2C4-VL | TGSRSATDFSLTISNVQAEDLADYLCGQSYDSPYTFGGGTKLEIK |
| SEQ ID NO: 351- | QVQLQQSGPQLVRPGASVKISCKASGYSFTNHWMHWMKQRPGQLGEWIGMIDPSDSETRLNQ |
| 98E2E7/98E2E12-VH | QFKDKATLTVDKSSSTAYMQLSSPTSEDSAVFYCARLGRYYFDYWGQGTTLTVSS |
| SEQ ID NO: 352- | NIVMTQSPKSMSVSVGERVTLSCRASDIVGTYVSWYQQKPEQSPKLLIYGASNRYTGVPDRF |
| 98E2E7/98E2E12-VL | TGSRSATDFSLTISNVQAEDLADYLCGQSYDSPYTFGGGTKLEIK |
| SEQ ID NO: 353- | QVQLQQSGPQLVRPGASVKISCKASGYSFTNYWMHWMKQRPGQGLEWIGMIDSDSETRLNQQ |
| 98G5F6/98G5F11-VH | FKDKATLTVDKSSSTAYMQLSSPTSEDSAVYYCARLGRYYFDFWGQGTTLTVSS |
| SEQ ID NO: 354- | NIVMTQSPKSMSVSVGERVTLSCRASEIVGTYVSWYQQKPEQSPKLLIYGASNRFTGVPDRF |
| 98G5F6/98G5F11-VL | TGSRSATDFSLTISNVQAEDLADYLCGQSYDSPYTFGGGTKLEIK |
| SEQ ID NO: 355- | EVQLQQSGAEFVKPGASVKLSCTASGFNIEDTYMHWVKQRPEQGLEWIGMIDPANGKTKYGP |
| 107F11B11/107F11C4/ | RFQDKATVTADTSSNTANLQLSSLTSEDTAVYYCADGIGYYVGAMDYWGQGTSVTVSS |
| 107F11F10-VH | |
| SEQ ID NO: 356- | DIQMNQSPSSLSASLGDTITITCHASQNINVWLSWYQQKPGNIPKLLIYKASNLHTGVPSRF |
| 107F11B11/107F11C4/ | SGSGSGTGFTLTISSLQPEDIATYYCQQGHSYPYTFGGGTKLEIK |
| 107F11F10-VL | |
| SEQ ID NO: 357- | QMQLQQSGPQLVRPGASVKISCKTSGYSFTHHWIHWMKQRPGQGLEWIGMIDPSDSETRLSQ |
| 108C10A6/108C10F5-VH | KFKDKATLTVDASSSTAYMQLNSPTSEDSALYFCARLGRYYFDYWGQGTTLTVSS |
| SEQ ID NO: 358- | NIVMTQSPRSMSMSVGERVTLSCKASENVGTYISWYQQKPDQSPKLLIYGASNRYTGVPDRF |
| 108C10A6/108C10F5-VL | TGSGSGTDFTLTISTVQAEDLADYHCGESYGHLYTFGGGTKLEIK |
| SEQ ID NO: 359- | QVQLQQSGPQLVRPGASVKISCKASGYSFTNNWMHWMKQRPGQGLEWIGMIDPSDSETRLNQ |
| 112E5D9/112E5F2/112E5H7-VH | QFRDKATLTVDKTSSTAYMQLSSPTSEDSAVYYCARLGRYYFDYWGLGTTLVTVSS |
| SEQ ID NO: 360- | NIVMTQSPKSMSVSVGERVTMNCRASEIVGTYVSWYQQKPEQSPKLLIYGAFNRYTGVPDRF |
| 112E5D9/112E5F2/112E5H7-VL | TGSRSGTDFSLNISNVQAEDLADYLCGQSYDSPYTFGGGTKLEIK |
| SEQ ID NO: 361- | QMQLQQSGPQLVRPGASVKISCKTSGYSFTHHWIHWMKQRPGQGLEWIGMIDPSDSETRLSQ |
| 108VH | KFKDKATLTVDASSSTAYMQLNPSTSEDSALYFCARLGRYYFDYWGQGTTLTVSS |
| SEQ ID NO: 362- | QMQLQQSGPQLVRPGASVKISCKTSGYSFTHHWIHWMKQRPGQGLEWIGIIDPSDSETRLSQ |
| 108VH.M1 | KFKDKATLTVDASSSTAYMQLNSPTSEDSALYFCARLGRYYFDYWGQGTTLTVSS |
| SEQ ID NO: 363- | QMQLQQSGPQLVRPGASVKISCKTSGYSFTHHWIHWMKQRPGQGLEWIGMIEPSDSETRLSQ |
| 108VH.M2 | KFKDKATLTVDASSSTAYMQLNSPTSEDSALYFCARLGRYYFDYWGQGTTLTVSS |
| SEQ ID NO: 365- | QMQLQQSGPQLVRPGASVKISCKTSGYSFTHHWIHWMKQRPGQGLEWIGMIDASDSETRLSQ |
| 108VH.M4 | KFKDKATLTVDASSSTAYMQLNSPTSEDSALYFCARLGRYYFDYWGQGTTLTVSS |
| SEQ ID NO: 366- | QVQLVQSGAEVKKPGSSVKVSCKASGYSFTHHWIHWVRQAPGQGLEWMGMIDPSDSETRLSQ |
| 108VH1 | KFKDRVTITADKSTSTAYMELSSLRSEDTAVYYCARLGRYYFDYWGQGTTVTVSS |
| SEQ ID NO: 367- | EVQLVQSAGEVKKPGSSVKVSCKASGYSFTHHWIHWMRQAPGQGLEWIGMIDPSDSETRLSQ |
| 108VH2 | KFKDRVTITADKSTSTAYMELSLRSEDTAVYFCARLGRYYFDYWGQGTTVTVSS |
| SEQ ID NO: 368- | EVQLVQSAGEVKKPGSSVKVSCKASGYSFTHHWIHWMRQAPGQGLEWIGMIDPSDSETRLSQ |
| 108VH3 | KFKDRATLTVDKSTSTAYMELSSLRSEDTAVYFCARLGRYYFDYWGQGTTVTVSS |
| SEQ ID NO: 369- | EVQLVQSGAEVKKPGSSVKVSCKTSGYSFTHHWIHWMKQAPGQGLEWIGMIDPSDSETRLSQ |
| 108VHa | KFKDKATLTVDKSTSTAYMELSSLRSEDTAVYFCARLGRYYFDYWGQGTTVTVSS |
| SEQ ID NO: 370- | EVQLVQSGAEVKKPGSSVKVSCKASGYSFTHHWIHWVRQAPGQGLEWMGMIDPSDSETRLSQ |
| 108VH4 | KFKDRVTITADKSTSTAYMELSSLRSEDTAVYYCARLGRYYFDYWGQGTTVTVSS |
| SEQ ID NO: 371- | EVQLVQSGAEVKKPGSSVKVSCKASGYSFTHHWIHWVRQAPGQGLEWMGMIEPSDSETRLSQ |
| 108VH4.M2 | KFKDRVTITADSSTSTAYMELSSLRSEDTAVYYCARLGRYYFDYWGQGTTVTVSS |
| SEQ ID NO: 372- | EVQLVQSGAEVKKPGSSVKVSCKASGYSTHHHWIHWVRQAPGQGLEWMGMISPSDSETRLSQ |
| 108VH4.M3 | KFKDRVTITADKSTSTAYMELSSLRSEDTAVYYCARLGRYYFDYWGQGTTVTVSS |
| SEQ ID NO: 373- | EVQLVQSGAEVKKPGSSVKVSCKASGYSTHHHWIHWVRQAPGQGLEWMGMIDASDSETRLSQ |
| 108VH4.M4 | KFKDRVTITADKSTSTAYMELSSLRSEDTAVYYCARLGRYYFDYWGQGTTVTVSS |
| SEQ ID NO: 374- | NIVMTQSPRSMSMSVGERVTLSCKASENVGTYISWYQQKPDQSPKLLIYGASNRYTGVPDRF |
| 108VL | TGSGSGTDFTLTISTVQAEDLADYHCGESYGHLYTFGGGTKLEIK |
| SEQ ID NO: 375- | EIVLTQSPATLSLSPGERATLSCKASENVGTYISWYQQKPGQAPRLLIYGASNRYTGIPARF |
| 108VL1 | GSGSGTDFTLTISSLEPEDFAVYYCGESYGHLYTFGGGTKVEIK |
| SEQ ID NO: 376- | EIVLTQSPATLSLSPGERATLSCRASENVGTYISWYQQKPGQAPRLLIYGASNRYTGIPARF |
| 108VL.M1 | SGSGSGTDFTLTISSLEPEDFAVYYCGESYGHLYTFGGGTKVEIK |
| SEQ ID NO: 377- | EIVLTQSPATLSLSPGERATLSCRASENVGTYISWYQQKPGQAPRLLIYGASNRYTGIPARF |
| 108VL1.M1 | SGSGSGTDFTLTISSLEPEDFAVYYCGESYGHLYTFGGGTKVEIK |
| SEQ ID NO: 378- | EIVLTQSPATLSLSPGERATLSCRASENVGTYISWYQQKPGQAPRLLIYGASNRYTGVPARF |
| 108VL2.M1 | SGSGSGTDFTLTISSLEPEDFAVYHCGESYGHLYTFGGGTKVEIK |
| SEQ ID NO: 379- | EIVLTQSPATLSLSPGERATLSCRASENVGTYISWYQQKPGQAPRLLIYGASNRYTGVPARF |
| 108VL3.M1 | SGSGSGTDFTLTISSVEPEDFAVYHCGESYGHLYTFGGGTKLEIK |
| SEQ ID NO: 380- | EVQLQQSGAEFVKPGASVKLSCTASGFNIEDTYMHWVKQRPEQGLEWIGMIDPANGKTKYGP |
| 107VH | RFQDKATVTADTSSNTANLQLSSLTSEDTAVYYCADGIGYYVGAMDYWGQGTSVTVSS |
| SEQ ID NO: 381- | EVQLVQSGAEVKKPGSSVKVSCKASGFNIEDTYMHWVRQAPGQGLEWMGMIDPANGKTKYGP |
| 108VH1 | RFQDRVTITADKSTSTAYMELSSLRSEDTAVYYCARGIGYYVGAMDYWGQGTTVTVSS |
| SEQ ID NO: 382- | EVQLVQSGAEVKKPGSSVKVSCKASGFNIEDTYMHWVRQAPGQGLEWIGMIDPANGKTKYGP |
| 107VH2 | RFQDRVTITADKSTSTAYMELSSLRSEDTAVYYCADGIGYYVGAMDYWGQGTTVTVSS |
| SEQ ID NO: 383- | EVQLVQSGAEVKKPGSSVKVSCKASGFNIEDTYMHWVRQAPGQGLEWIGMIDPANGKTKYGP |
| 107VH3 | RFQDRVTVTADKSTSTAYMELSSLRSEDTAVYYCADGIGYYVGAMDYWGQGTTVTVSS |
| SEQ ID NO: 384- | DIQMNQSPSSLSASLGDTITITCHASQNINVWLSWYQQKPGNIPKLLIYKASNLHTGVPSRF |
| 107VL | SGSGSGTGFTLTISSLQPEDIATYYCQQGHSYPYTFGGGTKLEIK |
| SEQ ID NO: 385- | DIQMTQSPSSLSASVGDRVTITCHASQNINVWLSWYQQKPGKAPKLLIYKASNLHTGVPSRF |
| 107VL1 | SGSGSGTDFTLTISSLQPEDFATYYCQQGHSYPYTFGGGTKVEIK |
| SEQ ID NO: 386- | DIQMTQSPSSLSASVGDRVTITCRASQNINVWLSWYQQKPGKAPKLLIYKASNLHTGVPSRF |
| 107VL1-M1 | SGSGSGTDFTLTISSLQPEDFATYYCQQGHSYPYTFGGGTKVEIK |
| SEQ ID NO: 387- | DIQMTQSPSSLSASVGDRITITCRASQNINVWLSWYQQKPGKAPKLLIYKASNLHTGVPSRF |
| 107VL2.M1 | SGSGSGTDFTLTISSLQPEDFATYYCQQGHSYPYTFGGGTKLEIK |
| SEQ ID NO: 388- | QVQLQQSGPQLVRPGASVKISCKASGYSFTNHWMHWMKQRPGQGLEWIGMIDPSDSETRLNQ |
| 98VH | QFKDKATLTVDKSSSTAYMQLSSPTSEDSAVFYCARLGRYYFDYWGQGTTLTVSS |
| SEQ ID NO: 389- | QVQLVQSGAEVKKPGSSVKVSCKASGYSFTNHWMHWVRQAPGQGLEWMGMIDPSDSETRLNQ |
| 98VH1 | QFKDRVTITADKSTSTAYMELSSLRSEDTAVYYCARLGRYYFDYWGQGTTVTVSS |
| SEQ ID NO: 390- | EVQLVQSGAEVKKPGSSVKVSCKASGYSFTNHWMHWMRQAPGQGLEWIGMIDPSDSETRLNQ |
| 98VH2 | QFKDRVTITADKSTSTAYMELSSLRSEDTAVFYCARLGRYYFDYWGQGTTVTVSS |
| SEQ ID NO: 391- | EVQLVQSGAEVKKPGSSVKVSCKASGYSFTNHWMHWMRQAPGQGLEWIGMIDPSDSETRLNQ |
| 98VH3 | QFKDRATLTVDKSTSTAYMELSSLRSEDTAVFYCARLGRYYFDYWGQGTTVTVSS |
| SEQ ID NO: 392- | EVQLVQSGAEVKKPGSSVKVSCKASGYSFTNHWMHWMKQAPGQGLEWIGMIDPSDSETRLNQ |
| 98VHa | QFKDKATLTVDKSTSTAYMELSSLRSEDTAVYYCARLGRYYFDYWGQGTTVTVSS |
| SEQ ID NO: 393- | NIVMTQSPKSMSVSVGERVTLSCRASDIVGTYVSWYQQKPEQSPKLLIYGASNRYTGVPDRF |
| 98VL | TGSRSATDFSLTISNVQAEDLADYLCGQSYDSPYTFGGGTKLEIK |
| SEQ ID NO: 394- | EIVMTQSPATLSCSPGERATLSCRASDIVGTYVSWYQQKPGQAPRLLIYGASNRTYGIPARF |
| 98VL1 | SGSGSGTEFTLTISSLQSEDFAVYYCGQSYDSPYTFGGGTKVEIK |
| SEQ ID NO: 395- | EIVMTQSPATLSCSPGERATLSCRASDIVGTYVSWYQQKPGQAPRLLIYGASNRTYGIPARF |
| 98VL2 | SGSGSGTEFTLTISSLQSEDFAVYYCGQSYDSPYTFGGGTKVEIK |
| SEQ ID NO: 396- | EIVMTQSPATLSCSPGERATLSCRASDIVGTYVSWYQQKPGQAPRLLIYGASNRTYGIPARF |
| 98VL3 | SGSGSGTEFTLTISSLQSEDFAVYYCGQSYDSPYTFGGGTKLEIK |
| SEQ ID NO: 397- | QMQLQQSGPQLVRPGASVKISCKTSGYSFTHHWIHWMKQRPGQGLEWIGMIDPSDSETRLSQ |
| 108C10A6_VH-huIgG1CH | KFKDKATLTVDASSSTAYMQLNSPTSEDSALYFCARLGRYYFDYWGQGTTLTVSSASTKGPS |
| | VFPLAPSSKSTSGGTAALGCLVKDYFPEPVTVSWNSGALTSGVHTFPAVLQSSLGYSLSSVV |
| | TVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVFSCSVMHEALHN |
| | HYTQKSLSLSPGK |
| SEQ ID NO: 398- | NIVMTQSPRSMSMSVGERVTLSCKASENVGTYISWYQQKPDQSPKLLIYGASNRYTGVPDRF |
| 108C10A6_VL-hIgKCL | TGSGSGTDFTLTISTVQAEDLADYHCGESYGHLYTFGGGTKLEIKRTVAAPSVFIFPPSDEQ |
| | LKSGTASVVCLLNNFYPREAKVQWKVDNALQSGNSQESVTEQDSKDSTYSLSSTLTLSKADY |
| | EKHKVYACEVTHQGLSSPVTKSFNRGEC |
| SEQ ID NO: 399- | EVQLVQSGAEVKKPGSSVKVSCKASGYSFTHHWIHWVRQAPGQGLEWMGMIDASDSETRLSQ |
| 108VH4.M4-huIgG1 | KFKDRVTITADKSTSTAYMELSSLRSEDTAVYYCARLGRYYFDYWGQGTTVTVSSASTKGPS |
| | VFPLAPSSKSTSGGTAALGCLVDKYFPEPVTVSWNSGALTSGVHTFPAVLQSSGLYSLSSVV |
| | TVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSRDELTKNQVSLTCLVKGF |
| | YPSDIAVEWESNGQEPNNYKTTPPVLDSDGSFFLYSKLTVDKSRWQQGNVFSCSVMHEALHN |
| | HYTQKSLSLSPGK |
| SEQ ID NO: 400- | EVQLVQSGAEVKKPGSSVKVSCKASGYSFTHHWIHWVRQAPGQGLEWMGMIDASDSETRLSQ |
| 108VH4.M4-hIgG2 | KFKDRVTITADKSTSTAYMELSSLRSEDTAVYYCARLGRYYFDYWGQGTTVTVSSASTKGPS |
| | VFPLAPCSRSTSESTAALGCLVKDYFPEPVTVSWNSGALTSGVHTFPAVLQSSGLYSLSSVV |
| | TVPSSNFGTQTYTCNVDHKPSNTKVDKTVERKCCVECPPCPAPPVAGPSVFLFPPKPKDTLM |
| | ISRTPEVTCVVVDVSHEDPEVQFNWYVDGVEVHNAKTKPREEQFNSTFRVVSVLTVVHQDWL |
| | NGKEYKCKVSNKGLPAPIEKTISKTKGQPREPQVYTLPPSREEMTKNQVSLTCLVKGFYPSD |
| | IAVEWESNGQPENNYKTTPPMLDSDGSFFLYSKLTVDKSRWQQGNVFSCSVMHEALHNHYTQ |
| | KSLSLSPGK |
| SEQ ID NO: 401- | EVQLVQSGAEVKKPGSSVKVSCKASGYSFTHHWIHWVRQAPGQGLEWMGMIDASDSETRLSQ |
| 108VH4.M4-hIgG4PE | KFKDRVTITADKSTSTAYMELSSLRSEDTAVYYCARLGRYYFDYWGQGTTVTVSSASTKGPS |
| | VFPLAPCSRSTSESTAALGCLVKDYFPEPVTVSWNSGALTSGVHTFPAVLQSSLGYSLSSVV |
| | TVPSSSLGTKTYTCNVDHKPSNTKVDKRVESKYGPPCPPCPAPEFEGGPSVFLFPPKPKDTL |
| | MISRTPEVTCVVVDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLTCLVKGFYPS |
| | DIAVEWESNGQPENNYKTTPPVLDSDGSFFLYSRLTVDKSRWQEGNVFSCSVMHEALHNHYT |
| | QKSLSLSLGK |
| SEQ ID NO: 402- | EIVLTQSPATLSLSPGERATLSCRASENVGTYISWYQQKPGQAPRLLIYGASNRYTGIPARF |
| 108VL1.M1-hIgKCL | SGSGSGTDFTLTISSLEPEDFAVYYCGESYGHLYTFGGGTKVEIKRTVAAPSVFIFPPSDEQ |
| | LKSGTASVVCLLNNFYPREAKVQWKVDNALQSGNSQESVTEQDSKDSTYSLSSTLTLSKADY |
| | EKHKVYACEVTHQGLSSPVTKSFNRGEC |
| SEQ ID NO: 403- | EVQLVQSGAEVKKPGSSVKVSCKASGYSFTHHWIHWVRQAPGQGLEWMGMIDADSEETRSLQ |
| 108VH4.M4-hIgG4- | KFKDRVTITADKSTSTAYMELSSLRSEDTAVYYCARLGRYYFDYWGQGTTVTVSSASTKGPS |
| | VFPLAPCSRSTSESTAALGCLVKDYFPEPVTVSWNSGALTSGVHTFPAVLQSSLGYSLSSVV |
| | TVPSSSLGTKTYTCNVDHKPSNTKVDKRVESKYGPPCPPCPAPEFLGGPSVFLFPPKPKDTL |
| | MISRTPECTCVVVDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLTCLVKGFYPS |
| | DIAVEWESNGQPENNYKTTPPVLDSDGSFFLYYSRLTVDKSRWQEGNVFSCSVMHEALHNHY |
| | TQKSLSLSLGK |
| SEQ ID NO: 404- | QVQLQQSGPQLVRPGASVKISCKASGYSFTNHWMHWMKQRPGQGLEWIGMIDPSDSETRLNQ |
| 98E2E12_VH-huIgG1CH | QFKDKATLTVDKSSSTAYMQLSSPTSEDSAVFYCARLGRYYFDYWGQGTTLTVSSASTKGPS |
| | VFPLAPSSKSTSGGTAALGCLVKDYFPEPVTVSWNSGALTSGVHTFPAVLQSSGLYSLSSVV |
| | TVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVFSCSVMHEALHN |
| | HYTQKSLSLSPGK |
| SEQ ID NO: 405- | NIVMTQSPKSMSVSVGERVTLSCRASDIVGTYVSWYQQKPEQSPKLLIYGASNRYTGGPDRF |
| 98E2E12_VL-hIgKCL | TGSRSATDFSLTISNQAEDLADYLCGQSYDSPYTFGGGTKLEIKRTVAAPSVFIFPPSDEQL |
| | KSGTASVVCLLNNFYPREAKVQWKVDNALQSGNSQESVTEQDSKDSTYSLSSTLTLSKADYE |
| | KHKVYACEVTHQGLSSPVTKSNFRGEC |
| SEQ ID NO: 406- | EVQLQQSGAEFVKPGASVKLSCTASGFNIEDTYMHWVKQRPEQGLEWIGMIDPANGKTKGPR |
| 107F11F10-VH-huIgG1CH | FQDKATVTADTSSNTANLQLSSLTSEDTAVYYCADGIGYYVGAMDYWGQGTSVTVSSASTKG |
| | PSVFPLAPSSKSTSGGTAALGCLVKDYFPEPVTVSWNSGALTSGVHTFPAVLQSSLGYSLSS |
| | VVTVPSSSLGTQTYICNVNHKPSNTKVDKKVEPKSCDKTHTCPPCPAPELLGGPSVFLFPPK |
| | PKDTLMISRTPECTCVVVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSRDELTKNQVSLTCLVK |
| | GFYPSDIAVEWESNGQPENNYKTTPPVLDSDGSFFLYSKLTVDKSRWQQGNVFSCSVMHEAL |
| | HNHYTQKSLSLSPGK |
| SEQ ID NO: 407- | DIQMNQSPSSLSASLGDTITITCHASQNINVWLSWYQQKPGNIPKLLIYKASNLHTGVPSRF |
| 107F11F10-VL-hIgKCL | SGSGSGTGFTLTISSLQPEDIATYYCQQGHSYPYTFGGGTKLEIKRTVAAPSVFIFPPSDEQ |
| | LKSGTASVVCLLNNFYPREAKVQWKVDNALQSGNSQESVTEQDSKDSTYSLSSTLTLSKADY |
| | EKHKVYACEVTHQGLSSPVTKSFNRGEC |
| SEQ ID NO: 412- | MWPLVAALLLGSACCGSAQLLFNKTKSVEFTFCNDTVVIPCFVTNMEAQNTTEVYVKWKFKG |
| human CD47 | RDIYTFDGALNKSTVPTDFSSAKIEVSQLLKGDASLKMDKSDAVSHTGNYTCEVTELTREGE |
| | TIIELKYRVVSWFSPNENILIVIFPIFAILLFWGQFGIKTLKYRSGGMDEKTIALLVAGLVI |
| | TVIVIVGAILFVPGEYSLKNATGLGLIVTSTGILILLHYYVFSTAIGLTSFVIALILVIQVI |
| | AYILAVVGLSLCIAACIPMHGPLLISGLSILALAQLLGLVYMKFVASNQKTIQPPRKAVEEP |
| | LNAFKESKGMMNDE |

NUCLEIC ACID SEQUENCES OF EXEMPLARY ANTIBODIES OF THIS DISCLOSURE
VH 108VH4.M4_VL1.M1-hIgG2
Set 1:
>108VH4.M4-hIgG2 (SEQ ID NO: 413)
GAGGTGCAGCTGGTGCAGTCCGGAGCTGAGGTGAAGAAGCCAGGATCCAGCGTGAAGGTGAGCTGCAAGGCTAGCGGCTACTCTTTCACC
CACCATTGGATCCACTGGGTGAGGCAGGCTCCTGGACAGGGACTGGAGTGGATGGGCATGATCGACGCTTCCGATAGCGAGACAAGACTG
TCTCAGAAGTTTAAGGACCGCGTGACCATCACAGCCGATAAGTCTACCTCCACAGCTTACATGGAGCTGTCTTCCCTGAGATCCGAGGAC
ACCGCCGTGTACTATTGTGCTAGGCTGGGCCGGTACTATTTCGATTATTGGGGCCAGGGCACCACAGTGACAGTGAGCTCTGCCTCCACC
AAGGGACCTAGCGTGTTTCCCCTGGCTCCTTGCAGCCGGTCTACATCCGAGAGCACCGCCGCTCTGGGATGTCTGGTGAAGGATTATTTC
CCTGAGCCAGTGACAGTGTCTTGGAACTCCGGCGCCCTGACAAGCGGAGTGCACACCTTTCCAGCTGTGCTGCAGTCTTCCGGCCTGTAT
TCTCTGAGCTCTGTGGTGACCBGTGCCTTCCAGCAATTTCGGCACCCAGACATACACCTGCAACGTGGACCATAAGCCATCCAATACAAA
GGTGGATAAGACCGTGGAGAGAAAGTGCTGCGTGGAGTGCCCACCTTGTCCTGCTCCACCAGTGGCTGGACCAAGCGTGTTCCTGTTTCC
TCCAAAGCCCAAGGACACACTGATGATCTCCCGCACACCTGAGGTGACCTGCGTGGTGGTGGACGTGAGCCACGAGGATCCCGAGGTGCA
GTTTAACTGGTACGTGGATGGCGTGGAGGTGCATAATGCTAAGACCAAGCCTAGGGAGGAGCAGTTCAACTCTACATTTCGGGTGGTGTC
CGTGCTGACCGTGGTGCACCAGGACTGGCTGAACGGCAAGGAGTACAAGTGCAAGGTGTCTAATAAGGGCCTGCCCGCTCCTATCGAGAA
GACAATCTCCAAGACCAAGGGCCAGCCAAGAGAGCCCCAGGTGTATACCCTGCCCCCTAGCCGCGAGGAGATGACAAAGAACCAGGTGTC
TCTGACCTGTCTGGTGAAGGGCTTCTACCCATCTGACATCGCCGTGGAGTGGGAGTCCAATGGCCAGCCCGAGAACAATTATAAGACCAC
ACCACCCATGCTGGACAGCGATGGCTCTTTCTTTCTGTACAGCAAGCTGACAGTGGATAAGTCTAGGTGGCAGCAGGGCAACGTGTTTTC
TTGCTCCGTGATGCATGAGGCTCTGCACAATCATTACACCCAGAAGAGCCTGTCTCTGTCCCCTGGCAAG
> VL 108CL.1M1-hIgKCL (SEQ ID NO: 414)
GAGATCGTGCTGACCCAGTCTCCAGCCACACTGTCTCTGTCCCCAGGAGAGAGGGCCACCCTGAGCTGCCGGGCTTCTGAGAACGTGGGC
ACATACATCTCCTGGTATCAGCAGAAGCCAGGACAGGCTCCTAGGCTGCTGATCTACGGCGCTAGCAATAGATATACCGGCATCCCTGCT
CGCTTCAGCGGATCTGGATCCGGCACAGACTTTACCCTGACAATCTCCAGCCTGGAGCCAGAGGATTTCGCCGTGTACTATTGTGGCGAG
TCCTACGGCCACCTGTATACCTTTGGCGGCGGCACAAAGGTGGAGATCAAGCGAACGGTGGCTGCACCATCTGTCTTCATCTTCCCGCCA
TCTGATGAGCAGTTGAAATCTGGAACTGCCTCTGTTGTGTGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGACAGCAAGGACAGCACCTACAGCCTCAGCAGCACCCTGACG
CTGAGCAAAGCAGACTACGAGAAACACAAAGTCTACGCCTGCGAAGTCACCCATCAGGGCCTGAGCTCGCCCGTCACAAAGAGCTTCAAC
AGGGGAGAGTGT
Set 2:
> VH 108VH4.M4-hIgG2 (SEQ ID NO: 415)
GAGGTGCAGCTGGTGCAGAGCGGAGCAGAGGTGAAGAAGCCTGGCAGCTCCGTGAAGGTGTCCTGCAAGGCCTCCGGCTACTCTTTCACA
CACCACTGGATCCACTGGGTGCGGCAGGCACCAGGACAGGGACTGGAGTGGATGGGCATGATCGACGCCAGCGATTCCGAGACCAGGCTG
TCCCAGAAGTTTAAGGACCGCGTGACCATCACAGCCGATAAGTCTACCAGCACAGCCTACATGGAGCTGTCTAGCCTGAGGAGCGAGGAC
ACCGCCGTGTACTATTGTGCCCGGCTGGGCAGATACTATTTCGATTATTGGGGCCAGGGCACCACAGTGACAGTGCCTCTGCCTCCACCA
AGGGACCAAGCGTGTTCCCACTGGCACCATGCTCCCGCTCTACAAGCGAGTCCACCGCCGCCCTGGGATGTCTGGTGAAGGACTATTTCC
CTGAGCCAGTGACAGTGAGCTGGAACTCCGGCGCCCTGACATCTGGCGTGCACACCTTTCCTGCCGTGCTGCAGAGCTCCGGCCTGTACA
GCCTGTCTAGCGTGGTGACCGTGCCCTCCTCTAATTTCGGCACCCAGACATATACCTGCAACGTGGACCACAAGCCTTCCAATACAAAGG
TGGATAAGACCGTGGAGAGGAAGTGCTGCGTGGAGTGCCCACCTTGTCCAGCACCACCAGTGGCAGGCCCTAGCGTGTTCCTGTTTCCTC
CAAAGCCAAAGGACACACTGATGATCTCTAGAACACCCGAGGTGACCTGCGTGGTGGTGGACGTGAGCCACGAGGATCCAGAGGTGCAGT
TTAACTGGTACGTGGATGGCGTGGAGGTGCACAATGCCAAGACCAAGCCCCGGGAGGAGCAGTTCAACAGCACCTTCCGGGTGGTGTCCG
TGCTGACCGTGGTGCACCAGGATTGGCTGAACGGCAAGGAGTATAAGTGCAAGGTGTCCAATAAGGGCCTGCCCGCCCCTATCGAGAAGA
CAATCTCTAAGACCAAGGGCCAGCCTAGGGAGCCACAGGTGTACACCCTGCCCCCTAGCCGCGAGGAGATGACAAAGAACCAGGTGTCCC
TGACCTGTCTGGTGAAGGGCTTCTATCCTTCCGACATCGCCGTGGAGTGGGAGTCTAATGGCCAGCCAGAGAACAATTACAAGACCACAC
CACCCATGCTGGACTCTGATGGCAGCTTCTTTCTGTATTCTAAGCTGACAGTGGATAAGAGCAGATGGCAGCAGGGCAACGTGTTTTCTT
GCAGCGTGATGCACGAGGCCCTGCACAATCACTACACCCAGAAGTCCCTGTCTCTGAGCCCCGGCAA
> VL 108VL1.M1-hIgKCL (SEQ ID NO: 416)
GAGATCGTGCTGACCCAGTCCCCTGCCACACTGAGCCTGTCCCCAGGAGAGAGGGCCACCCTGTCTTGCAGAGCAAGCGAGAACGTGGGC
ACATACATCTCTTGGTATCAGCAGAAGCCAGGACAGGCACCAAGGCTGCTGATCTACGGAGCAAGCAATAGGTATACCGGCATCCCCGCA
CGCTTCTCTGGAAGCGGATCCGGCACAGACTTTACCCTGACAATCAGCTCCCTGGAGCCTGAGGATTTCGCCGTGTACTATTGCGGCGAG
AGCTACGGCCACCTGTATACCTTTGGCGGCGGCACAAAGGTGGAGATCAAGAGGACCGTGGCAGCACCAAGCGTGTTCATCTTTCCCCCT
TCCGACGAGCAGCTGAAGTCCGGCACCGCCTCTGTGGTGTGCCTGCTGAACAATTTCTACCCCAGAGAGGCCAAGGTGCAGTGGAAGGTG
GATAACGCCCTGCAGTCTGGCAATAGCCAGGAGTCCGTGACCGAGCAGGACTCTAAGGATAGCACATATTCCCTGTCTAGCACCCTGACA
CTGTCCAAGGCCGACTACGAGAAGCACAAGGTGTATGCATGCGAGGTGACCCACCAGGGACTGTCCTCTCCTGTGACAAAGTCTTTTAAC
AGAGGCGAGTGT
Set 3:
> VH 108VH4.M4-hIgG2 (SEQ ID NO: 417)
GAGGTGCAGCTGGTGCAGAGCGGAGCAGAGGTGAAGAAGCCTGGCAGCTCCGTGAAGGTGTCCTGCAAGGCCTCCGGCTACTCTTTCACA
ACACCACTGGATCCACTGGGTGCGGCAGGCACCAGGACAGGGACTGGAGTGGATGGGCATGATCGACGCCAGCGATTCCGAGACCAGGCT
GTCCCAGAAGTTTAAGGACCGCGTGACCATCACAGCCGATAAGTCTACCAGCACAGCCTACATGGAGCTGTCTAGCCTGAGGAGCGAGGA
CACCGCCGTGTACTATTGTGCCCGGCTGGGCAGATACTATTTCGATTATTGGGGCCAGGGCACCACAGTGACAGTGTCCTCTGCCTCCAC
CAAGGGCCCCTCTGTGTTTCCACTGGCCCCCTGCTCCAGGTCTACAAGCGAGTCCACCGCAGCACTGGGATGTCTGGTGAAGGACTATTT
CCCTGAGCCAGTGACAGTGAGCTGGAACTCCGGCGCCCTGACATCTGGCGTGCACACCTTTCCTGCCGTGCTGCAGAGCTCCGGCCTGTA
CAGCCTGTCTAGCGTGGTGACCGTGCCCTCCTCTAATTTCGGCACCCAGACATATACCTGCAACGTGGACCACAAGCCTTCCAATACAAA
GGTGGATAAGACCGTGGAGCGGAAGTGCTGTGTGGAGTGCCCACCTTGTCCAGCACCACCAGTGGCAGGCCCTAGCGTGTTCCTGTTTCC
TCCAAAGCCAAAGGACACACTGATGATCTCTAGAACACCCGAGGTGACCTGTGTGGTGGTGGACGTGAGCCACGAGGATCCAGAGGTGCA
GTTTAACTGGTACGTGGATGGCGTGGAGGTGCACAATGCCAAGACCAAGCCCCGGGAGGAGCAGTTCAACAGCACCTTCCGGGTGGTGTC
CGTGCTGACCGTGGTGCACCAGGATTGGCTGAACGGCAAGGAGTATAAGTGCAAGGTGTCCAATAAGGGCCTGCCCGCCCCTATCGAGAA
GACAATCTCTAAGACCAAGGGCCAGCCTAGGGAGCCACAGGTGTACACCCTGCCCCCTAGCCGCGAGGAGATGACAAAGAACCAGGTGTC
CCTGACCTGTCTGGTGAAGGGCTTCTATCCTTCCGACATCGCCGTGGAGTGGGAGTCTAATGGCCAGCCAGAGAACAATTACAAGACCAC
ACCACCCATGCTGGACTCTGATGGCAGCTTCTTTCTGTATTCTAAGCTGACAGTGGATAAGAGCAGATGGCAGCAGGGCAACGTGTTTTC
TTGCAGCGTGATGCACGAGGCCCTGCACAATCACTACACCCAGAAGTCCCTGTCTCTGAGCCCCGGCAAG
> VL 108VL.1M1-hIgKCL (SEQ ID NO: 418)
GAGATCGTGCTGACCCAGTCCCCTGCAACACTGAGCCTGTCCCCAGGAGAGAGGGCAACCCTGTCTTGCAGAGCAAGCGAGAACGTGGGC
ACATACATCTCTTGGTATCAGCAGAAGCCAGGACAGGCACCAAGGCTGCTGATCTACGGAGCAAGCAATAGGTATACCGGCATCCCCGCA
CGCTTCTCTGGAAGCGGATCCGGCACAGACTTTACCCTGACAATCAGCTCCCTGGAGCCTGAGGATTTCGCCGTGTACTATTGCGGCGAG
AGCTACGGCCACCTGTATACCTTTGGCGGCGGCACAAAGGTGGAGATCAAGAGGACCGTGGCAGCACCAAGCGTGTTCATCTTTCCCCCT
TCCGACGAGCAGCTGAAGTCCGGCACCGCCTCTGTGGTGTGTCTGCTGAACAATTTCTACCCCAGAGAGGCCAAGGTGCAGTGGAAGGTG
GATAACGCCCTGCAGTCTGGCAATAGCCAGGAGTCCGTGACCGAGCAGGACTCTAAGGATAGCACATATTCCCTGTCTAGCACCCTGACA
CTGTCCAAGGCCGACTACGAGAAGCACAAGGTGTATGCATGCGAGGTGACCCACCAGGGACTGTCCTCTCCTGTGACAAAGTCTTTTAAC
AGAGGCGAGTGT
108VH4.M4_VL1.M1-hIgG4PE
Set 1:
> VH 108VH4.M4-hIgG4PE (SEQ ID NO: 419)
GAGGTGCAGCTGGTGCAGTCCGGAGCTGAGGTGAAGAAGCCAGGATCCAGCGTGAAGGTGAGCTGCAAGGCTAGCGGCTACTCTTTCACC
CACCATTGGATCCACTGGGTGAGGCAGGCTCCTGGACAGGGACTGGAGTGGATGGGCATGATCGACGCTTCCGATAGCGAGACAAGACTG
TCTCAGAAGTTTAAGGACCGCGTGACCATCACAGCCGATAAGTCTACCTCCACAGCTTACATGGAGCTGTCTTCCCTGAGATCCGAGGAC
ACCGCCGTGTACTATTGTGCTAGGCTGGGCCGGTACTATTTCGATTATTGGGGCCAGGGCACCACAGTGACAGTGAGCTCTGCCAGCACA
AAGGGCCCTTCCGTGTTCCCACTGGCTCCCTGCTCCAGAAGCACATCTGAGTCCACCGCCGCTCTGGGCTGTCTGGTGAAGGACTACTTC
CCTGAGCCAGTGACCGTGTCCTGGAACAGCGGCGCCCTGACATCTGGCGTGCACACCTTTCCAGCTGTGCTGCAGTCCAGCGGCCTGTAC
TCCCTGTCTTCCGTGGTGACAGTGCCCAGCTCTTCCCTGGGCACCAAGACATATACCTGCAACGTGGACCATAAGCCTTCCAATACCAAG
GTGGATAAGAGGGTGGAGAGCAAGTACGGACCACCTTGCCCACCATGTCCAGCTCCTGAGTTTGAGGGAGGACCATCCGTGTTCCTGTTT
CCTCCAAAGCCTAAGGACACCCTGATGATCAGCCGGACACCTGAGGTGACCTGCGTGGTGGTGGACGTGTCTCAGGAGGATCCAGAGGTG
CAGTTCAACTGGTACGTGGATGGCGTGGAGGTGCACAATGCTAAGACCAAGCCAAGAGAGGAGCAGTTTAATTCCACATACCGCGTGGTG
AGCGTGCTGACCGTGCTGCATCAGGATTGGCTGAACGGCAAGGAGTATAAGTGCAAGGTGTCCAATAAGGGCCTGCCCAGCTCTATCGAG
AAGACAATCAGCAAGGCTAAGGGACAGCCTAGGGAGCCACAGGTGTACACCCTGCCCCCTTCTCAGGAGGAGATGACAAAGAACCAGGTG
TCCCTGACCTGTCTGGTGAAGGGCTTCTATCCAAGCGACATCGCTGTGGAGTGGGAGTCTAATGGCCAGCCCGAGAACAATTACAAGACC
ACACCACCCGTGCTGGACTCTGATGGCTCCTTCTTTCTGTATTCTAGGCTGACAGTGGATAAGTCCCGGTGGCAGGAGGGCAACGTGTTT
AGCTGCTCTGTGATGCACGAGGCCCTGCACAATCATTATACCCAGAAGTCCCTGAGCCTGTCTCTGGGCAAG
> VL 108CL.1M1-hIgKCL (SEQ ID NO: 414)
GAGATCGTGCTGACCCAGTCTCCAGCCACACTGTCTCTGTCCCCAGGAGAGAGGGCCACCCTGAGCTGCCGGGCTTCTGAGAACGTGGGC
ACATACATCTCCTGGTATCAGCAGAAGCCAGGACAGGCTCCTAGGCTGCTGATCTACGGCGCTAGCAATAGATATACCGGCATCCCTGCT
CGCTTCAGCGGATCTGGATCCGGCACAGACTTTACCCTGACAATCTCCAGCCTGGAGCCAGAGGATTTCGCCGTGTACTATTGTGGCGAG
TCCTACGGCCACCTGTATACCTTTGGCGGCGGCACAAAGGTGGAGATCAAGCGAACGGTGGCTGCACCATCTGTCTTCATCTTCCCGCCA
TCTGATGAGCAGTTGAAATCTGGAACTGCCTCTGTTGTGTGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGACAGCAAGGACAGCACCTACAGCCTCAGCAGCACCCTGACG
CTGAGCAAAGCAGACTACGAGAAACACAAAGTCTACGCCTGCGAAGTCACCCATCAGGGCCTGAGCTCGCCCGTCACAAAGAGCTTCAAC
AGGGGAGAGTGT
Set 2:
> VH 108VH4.M4-hIgG4PE (SEQ ID NO: 420)
GAGGTGCAGCTGGTGCAGTCTGGCGCCGAGGTGAAGAAGCCAGGCAGCTCCGTGAAGGTGTCCTGCAAGGCCTCCGGCTACTCTTTCACA
CACCACTGGATCCACTGGGTGCGGCAGGCACCAGGACAGGGACTGGAGTGGATGGGCATGATCGACGCCAGCGATTCCGAGACCCGGCTG
AGCCAGAAGTTTAAGGACAGAGTGACCATCACAGCCGATAAGTCTACCAGCACAGCCTACATGGAGCTGTCTAGCCTGAGGTCCGAGGAC
ACCGCCGTGTACTATTGTGCCCGGCTGGGCAGATACTATTTCGATTATTGGGGCCAGGGCACCACAGTGACAGTGTCCTCTGCCTCCACC
AAGGGACCAAGCGTGTTCCCACTGGCACCATGCTCCCGCTCTACAAGCGAGTCCACCGCCGCCCTGGGATGTCTGGTGAAGGACTATTTC
CCTGAGCCAGTGACCGTGAGCTGGAACTCCGGCGCCCTGACAAGCGGAGTGCACACCTTTCCTGCCGTGCTGCAGAGCTCCGGCCTGTAC
TCCCTGTCTAGCGTGGTGACAGTGCCCTCCTCTAGCCTGGGCACCAAGACATATACCTGCAACGTGGACCACAAGCCTAGCAATACCAAG
GTGGATAAGCGGGTGGAGTCCAAGTACGGACCACCTTGCCCACCATGTCCAGCACCTGAGTTCGAGGGAGGACCAAGCGTGTTCCTGTTT
CCTCCAAAGCCTAAGGACACACTGATGATCTCCAGAACACCTGAGGTGACCTGCGTGGTGGTGGACGTGTCTCAGGAGGATCCAGAGGTG
CAGTTCAACTGGTACGTGGATGGCGTGGAGGTGCACAATGCCAAGACCAAGCCTAGGGAGGAGCAGTTTAATAGCACATACCGCGTGGTG
TCCGTGCTGACCGTGCTGCACCAGGATTGGCTGAACGGCAAGGAGTATAAGTGCAAGGTGAGCAATAAGGGCCTGCCATCCTCTATCGAG
AAGACAATCTCCAAGGCCAAGGGCCAGCCTAGAGAGCCACAGGTGTACACCCTGCCCCCTTCTCAGGAGGAGATGACAAAGAACCAGGTG
AGCCTGACCTGTCTGGTGAAGGGCTTCTATCCATCCGACATCGCCGTGGAGTGGGAGTCTAATGGCCAGCCCGAGAACAATTACAAGACC
ACACCACCCGTGCTGGACTCTGATGGCAGCTTCTTTCTGTATTCTAGGCTGACAGTGGATAAGAGCCGCTGGCAGGAGGGCAACGTGTTT
TCTTGCAGCGTGATGCACGAGGCCCTGCACAATCACTACACCCAGAAGTCCCTGTCTCTGAGCCTGGGCAAG
> VL 108VL.1M1-hIgKCL (SEQ ID NO: 416)
GAGATCGTGCTGACCCAGTCCCCTGCCACACTGAGCCTGTCCCCAGGAGAGAGGGCCACCCTGTCTTGCAGAGCAAGCGAGAACGTGGGC
ACATACATCTCTTGGTATCAGCAGAAGCCAGGACAGGCACCAAGGCTGCTGATCTACGGAGCAAGCAATAGGTATACCGGCATCCCCGCA
CGCTTCTCTGGAAGCGGATCCGGCACAGACTTTACCCTGACAATCAGCTCCCTGGAGCCTGAGGATTTCGCCGTGTACTATTGCGGCGAG
AGCTACGGCCACCTGTATACCTTTGGCGGCGGCACAAAGGTGGAGATCAAGAGGACCGTGGCAGCACCAAGCGTGTTCATCTTTCCCCCT
TCCGACGAGCAGCTGAAGTCCGGCACCGCCTCTGTGGTGTGCCTGCTGAACAATTTCTACCCCAGAGAGGCCAAGGTGCAGTGGAAGGTG
GATAACGCCCTGCAGTCTGGCAATAGCCAGGAGTCCGTGACCGAGCAGGACTCTAAGGATAGCACATATTCCCTGTCTAGCACCCTGACA
CTGTCCAAGGCCGACTACGAGAAGCACAAGGTGTATGCATGCGAGGTGACCCACCAGGGACTGTCCTCTCCTGTGACAAAGTCTTTTAAC
AGAGGCGAGTGT
Set 3:
> VH 108VH4.M4-hIgG4PE (SEQ ID NO: 421)
GAGGTGCAGCTGGTGCAGTCTGGCGCCGAGGTGAAGAAGCCAGGCAGCTCCGTGAAGGTGTCCTGCAAGGCCTCCGGCTACTCTTTCACA
CACCACTGGATCCACTGGGTGCGGCAGGCACCAGGACAGGGACTGGAGTGGATGGGCATGATCGACGCCAGCGATTCCGAGACCCGGCTG
AGCCAGAAGTTTAAGGACAGAGTGACCATCACAGCCGATAAGTCTACCAGCACAGCCTACATGGAGCTGTCTAGCCTGAGGTCCGAGGAC
ACCGCCGTGTACTATTGTGCCCGGCTGGGCAGATACTATTTCGATTATTGGGGCCAGGGCACCACAGTGACAGTGTCCTCTGCCTCCACC
AAGGGCCCCTCTGTGTTTCCACTGGCCCCCTGCTCCAGGTCTACAAGCGAGTCCACCGCAGCACTGGGATGTCTGGTGAAGGACTATTTC
CCTGAGCCAGTGACCGTGAGCTGGAACTCCGGAGCACTGACAAGCGGAGTGCACACCTTTCCTGCCGTGCTGCAGAGCTCCGGCCTGTAC
TCCCTGTCTAGCGTGGTGACAGTGCCCTCCTCTAGCCTGGGCACCAAGACATATACCTGCAACGTGGACCACAAGCCTAGCAATACCAAG
GTGGATAAGCGGGTGGAGTCCAAGTACGGACCACCTTGCCCACCATGTCCAGCACCTGAGTTCGAGGGAGGACCAAGCGTGTTCCTGTTT
CCTCCAAAGCCTAAGGACACACTGATGATCTCCAGAACACCTGAGGTGACCTGTGTGGTGGTGGACGTGTCTCAGGAGGATCCAGAGGTG
CAGTTCAACTGGTACGTGGATGGCGTGGAGGTGCACAATGCCAAGACCAAGCCTAGGGAGGAGCAGTTTAATAGCACATACCGCGTGGTG
TCCGTGCTGACCGTGCTGCACCAGGATTGGCTGAACGGCAAGGAGTATAAGTGCAAGGTGAGCAATAAGGGCCTGCCATCCTCTATCGAG
AAGACAATCTCCAAGGCCAAGGGCCAGCCTAGAGAGCCACAGGTGTACACCCTGCCCCCTTCTCAGGAGGAGATGACAAAGAACCAGGTG
AGCCTGACCTGTCTGGTGAAGGGCTTCTATCCATCCGACATCGCCGTGGAGTGGGAGTCTAATGGCCAGCCCGAGAACAATTACAAGACC
ACACCACCCGTGCTGGACTCTGATGGCAGCTTCTTTCTGTATTCTAGGCTGACAGTGGATAAGAGCCGCTGGCAGGAGGGCAACGTGTTT
TCTTGCAGCGTGATGCACGAGGCCCTGCACAATCACTACACCCAGAAGTCCCTGTCTCTGAGCCTGGGCAAG
> VL 108VL1.M1-hIgKCL (SEQ ID NO: 418)
GAGATCGTGCTGACCCAGTCCCCTGCAACACTGAGCCTGTCCCCAGGAGAGAGGGCAACCCTGTCTTGCAGAGCAAGCGAGAACGTGGGC
ACATACATCTCTTGGTATCAGCAGAAGCCAGGACAGGCACCAAGGCTGCTGATCTACGGAGCAAGCAATAGGTATACCGGCATCCCCGCA
CGCTTCTCTGGAAGCGGATCCGGCACAGACTTTACCCTGACAATCAGCTCCCTGGAGCCTGAGGATTTCGCCGTGTACTATTGCGGCGAG
AGCTACGGCCACCTGTATACCTTTGGCGGCGGCACAAAGGTGGAGATCAAGAGGACCGTGGCAGCACCAAGCGTGTTCATCTTTCCCCCT
TCCGACGAGCAGCTGAAGTCCGGCACCGCCTCTGTGGTGTGTCTGCTGAACAATTTCTACCCCAGAGAGGCCAAGGTGCAGTGGAAGGTG
GATAACGCCCTGCAGTCTGGCAATAGCCAGGAGTCCGTGACCGAGCAGGACTCTAAGGATAGCACATATTCCCTGTCTAGCACCCTGACA
CTGTCCAAGGCCGACTACGAGAAGCACAAGGTGTATGCATGCGAGGTGACCCACCAGGGACTGTCCTCTCCTGTGACAAAGTCTTTTAAC
AGAGGCGAGTGT

### Other Embodiments

The foregoing description discloses only exemplary embodiments of the invention.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

1: An anti-CD47 antibody comprising at least one antibody-antigen binding site, said antibody binds human CD47, inhibits, blocks, antagonizes, neutralizes or otherwise interferes with CD47 expression, activity and/or signaling, and does not cause significant agglutination of cells, wherein said antibody is a human antibody, a chimeric antibody, a humanized antibody, a primatized antibody, a bi-specific antibody, a conjugated antibody, a Small Modular ImmunoPharmaceutical, a single chain antibody, a cameloid antibody, a CDR-grafted antibody, or an antigen-binding fragment or antigen binding functional variant thereof.

2: The antibody of claim 1, wherein said antibody does not cause hemagglutination of human red blood cells.

3: The antibody of claim 1, wherein said anti-CD47 antibody blocks the interaction between human CD47 and human signal-regulatory-protein α (SIRPα).

4-10. (canceled)

11: The antibody of claim 1, wherein said antibody does not promote clumping of CD47 positive cell lines.

12: The antibody of claim 1, wherein said antibody comprises a variable heavy chain selected from SEQ ID NOs: 349, 351, 353, 355, 357, 359, 361-373, 380-383, 388-392 and a variable light chain selected from SEQ ID NOs: 350, 352, 354, 356, 358, 360, 374-379, 384-387 and 393-396; or
wherein said antibody comprises a variable heavy chain that is at least 90%, 91%, 92%, 93%, 94%, 95%, 96%, 97%, 98%, 99% or more identical to a sequence set forth in one of SEQ ID NOs: 349, 351, 353, 355, 357, 359, 361-373, 380-383, 388-392, and a variable light chain that is at least 90%, 91%, 92%, 93%, 94%, 95%, 96%, 97%, 98%, 99% or more identical to a sequence set forth in one of SEQ ID NOs: 350, 352, 354, 356, 358, 360, 374-379, 384-387 and 393-396.

13-16. (canceled)

17: The antibody of claim 1, wherein said antibody comprises a constant region modified at amino acid Asn297, Leu235 and/or Leu234.

18-20. (canceled)

21: The antibody of claim 1, wherein said antibody comprises a human IgG₃ constant region that is modified at amino acid amino acid Arg435; or
wherein said antibody comprises a human IgG₄ constant region that is modified within the hinge region to prevent or reduce strand exchange.

22-26. (canceled)

27: The antibody of claim 1, wherein said antibody comprises a human IgG constant region modified to enhance FcRn binding, wherein said human IgG constant region has one or more amino acid modifications of Met252Tyr, Ser254Thr, Thr256Glu, Met428Leu or Asn434Ser (M252Y, S254T, T256E M428L, or N434S); and/or
wherein said antibody comprises a human IgG constant region modified to alter antibody-dependent cellular cytotoxicity (ADCC) and/or complement-dependent cytotoxicity (CDC); and/or
wherein said antibody comprises a human IgG constant region modified to induce heterodimerization, wherein said antibody has an amino acid modification of T366W or T366S and/or an amino acid modification of L368A, or Y407V, S354C, or Y349C.

28-29. (canceled)

30: The antibody of claim 1, wherein said antibody is a humanized or human antibody having a variable heavy chain region (V_{H}) and/or variable light (V_{L}) chain region selected from the group consisting of: SEQ ID NOs: 361-373, 375-379, 381-383, 385-387, 389-392, 394-396, 399-404; or
wherein said antibody is a humanized or human antibody having a variable heavy chain region (V_{H}) and/or variable light (V_{L}) chain region that at least 90%, 91%, 92%, 93%, 94%, 95%, 96%, 97%, 98%, 99% or more identical to a sequence selected from the group consisting of: SEQ ID NOs: 361-373, 375-379, 381-383, 385-387, 389-392, 394-396, 399-404.

31. (canceled)

32: The antibody of claim 1, wherein said antibody is a humanized antibody or a human antibody.

33. (canceled)

34: A vector comprising a nucleic acid encoding an antibody of claim 1; or comprising: a nucleic acid encoding a heavy chain region (V_{H}) of an antibody and/or variable light (V_{L}) chain region of an antibody that is at least 90%, 91%, 92%, 93%, 94%, 95%, 96%, 97%, 98%, 99% or more identical to a sequence selected from the group consisting of: SEQ ID NOs: 337-348, 413-421.

35-37. (canceled)

38: A method of treating, delaying the progression of, preventing relapse of, or alleviating a symptom of a cancer or other neoplastic condition in a human patient with cancer or other neoplastic condition, comprising administering to said patient a therapeutically effective amount of an antibody comprising at least one antibody-antigen binding site, said antibody binds human CD47, inhibits, blocks, antagonizes, neutralizes or otherwise interferes with CD47 expression, activity and/or signaling, and does not cause significant agglutination of cells or administering to said patient a therapeutically effective amount of a pharmaceutical composition comprising said antibody and a pharmaceutical excipient.

39: The method of claim 38, wherein said antibody is an antibody.

40-41. (canceled)

42: The method of claim 38, wherein said cancer or other neoplastic condition is selected from the group consisting of non-Hodgkin's lymphoma (NHL), acute lymphocytic leukemia (ALL), acute myeloid leukemia (AML), chronic lymphocytic leukemia (CLL), chronic myelogenous leukemia (CML), multiple myeloma (MM), breast cancer, ovarian cancer, head and neck cancer, bladder cancer, melanoma, colorectal cancer, pancreatic cancer, lung cancer, leiomyoma, leiomyosarcoma, glioma, glioblastoma, breast tumors, ovarian tumors, lung tumors, pancreatic tumors, prostate tumors, melanoma tumors, colorectal tumors, lung tumors, head and neck tumors, bladder tumors, esophageal tumors, liver tumors, kidney tumors and hematological cancer.

43-44. (canceled)

45: The method of claim 42, wherein said hematological cancer is leukemia, lymphoma or myeloma.

46: The method of claim 42, wherein said hematological cancer is a leukemia selected from the group consisting of acute lymphocytic leukemia (ALL), acute myeloid leukemia (AML), chronic lymphocytic leukemia (CLL), chronic myelogenous leukemia (CML), Myeloproliferative disorder/neoplasm (MPDS), and myelodysplasia syndrome; or
wherein said hematological cancer is a lymphoma selected from the group consisting of a Hodgkin's lymphoma, both indolent and aggressive non-Hodgkin's lymphoma, Burkitt's lymphoma, and follicular lymphoma (small cell and large cell); or
wherein said hematological cancer is a myeloma selected from the group consisting of multiple myeloma (MM), giant cell myeloma, heavy-chain myeloma, and light chain or Bence-Jones myeloma.

47-48. (canceled)

49: The method of claim 38, further comprising administering one or more additional agents to said patient.

50: The method of claim 49, wherein said additional agent is a therapeutic agent, or an anti-cancer agent.

51. (canceled)

52: The antibody of claim 1, wherein the said antibody comprising:
(a) a heavy chain variable domain (V_{H}) comprising
i. a heavy chain CDR1 comprising an amino acid sequence selected from the group consisting of SEQ ID NOs: 49, 51, 53, 55, 57, 59, 62-65, 86-87;
ii. a heavy chain CDR2 comprising an amino acid sequence selected from the group consisting of SEQ ID NOs: 145, 147, 149, 151, 153, 155, 158-161, 182-183; and
iii. a heavy chain CDR3 comprising an amino acid sequence selected from the group consisting of SEQ ID NOs: 241, 243, 245, 247, 249, 251, 254-257, 278-279 and
(b) a light chain variable domain (V_{L}) comprising, respectively,
i. a light chain CDR1 comprising an amino acid sequence selected from the group consisting of SEQ ID NOs: 50, 52, 54, 56, 58, 60, 76-79;
ii. a light chain CDR2 comprising an amino acid sequence selected from the group consisting of SEQ ID NOs: 146, 148, 150, 152, 154, 156, 172-175; and
iii. a light chain CDR3 comprising an amino acid sequence selected from the group consisting of SEQ ID NOs: 242, 244, 246, 248, 250, 252, 268-271.

53: The antibody of claim 52, wherein the said antibody comprise any one of the following:
(1) a V_{H} comprises the heavy chain CDR1, CDR2 and CDR3 sequences having the amino acid sequences of SEQ ID NOs: 49, 145 and 241, respectively, and a V_{L} comprises the light chain CDR1, CDR2 and CDR3 having the amino acid sequences of SEQ ID NOs: 50, 146 and 242, respectively;
(2) a V_{H} comprises the heavy chain CDR1, CDR2 and CDR3 sequences having the amino acid sequences of SEQ ID NOs: 51, 147 and 243, respectively, and a V_{L} comprises the light chain CDR1, CDR2 and CDR3 having the amino acid sequences of SEQ ID NOs: 52, 148 and 244, respectively;
(3) a V_{H} comprises the heavy chain CDR1, CDR2 and CDR3 sequences having the amino acid sequences of SEQ ID NOs: 53, 149 and 245, respectively, and a V_{L} comprises the light chain CDR1, CDR2 and CDR3 having the amino acid sequences of SEQ ID NOs: 54, 150 and 246, respectively;
(4) a V_{H} comprises the heavy chain CDR1, CDR2 and CDR3 sequences having the amino acid sequences of SEQ ID NOs: 55, 151 and 247, respectively, and a V_{L} comprises the light chain CDR1, CDR2 and CDR3 having the amino acid sequences of SEQ ID NOs: 56, 152 and 248, respectively;
(5) a V_{H} comprises the heavy chain CDR1, CDR2 and CDR3 sequences having the amino acid sequences of SEQ ID NOs: 57, 153 and 249, respectively, and a V_{L} comprises the light chain CDR1, CDR2 and CDR3 having the amino acid sequences of SEQ ID NOs: 58, 154 and 250, respectively;
(6) a V_{H} comprises the heavy chain CDR1, CDR2 and CDR3 sequences having the amino acid sequences of SEQ ID NOs: 59, 155 and 251, respectively, and a V_{L} comprises the light chain CDR1, CDR2 and CDR3 having the amino acid sequences of SEQ ID NOs: 60, 156 and 252, respectively;
(7) a V_{H} comprises the heavy chain CDR1, CDR2 and CDR3 sequences having the amino acid sequences of SEQ ID NOs:62, 158 and 254, respectively, and a V_{L} comprises the light chain CDR1, CDR2 and CDR3 having the amino acid sequences of SEQ ID NOs: 76, 172 and 268, respectively;
(8) a V_{H} comprises the heavy chain CDR1, CDR2 and CDR3 sequences having the amino acid sequences of SEQ ID NOs:63, 159 and 255, respectively, and a V_{L} comprises the light chain CDR1, CDR2 and CDR3 having the amino acid sequences of SEQ ID NOs: 77, 173 and 269, respectively;
(9) a V_{H} comprises the heavy chain CDR1, CDR2 and CDR3 sequences having the amino acid sequences of SEQ ID NOs:64, 160 and 256, respectively, and a V_{L} comprises the light chain CDR1, CDR2 and CDR3 having the amino acid sequences of SEQ ID NOs: 78, 174 and 270, respectively;
(10) a V_{H} comprises the heavy chain CDR1, CDR2 and CDR3 sequences having the amino acid sequences of SEQ ID NOs:65, 161 and 257, respectively, and a V_{L} comprises the light chain CDR1, CDR2 and CDR3 having the amino acid sequences of SEQ ID NOs: 79, 175 and 271, respectively;
(11) a V_{H} comprises the heavy chain CDR1, CDR2 and CDR3 sequences having the amino acid sequences of SEQ ID NOs:65, 161 and 257, respectively, and a V_{L} comprises the light chain CDR1, CDR2 and CDR3 having the amino acid sequences of SEQ ID NOs: 76, 172 and 268, respectively;
(12) a V_{H} comprises the heavy chain CDR1, CDR2 and CDR3 sequences having the amino acid sequences of SEQ ID NOs:86, 182 and 278, respectively, and a V_{L} comprises the light chain CDR1, CDR2 and CDR3 having the amino acid sequences of SEQ ID NOs: 56, 152 and 248, respectively; and
(13) a V_{H} comprises the heavy chain CDR1, CDR2 and CDR3 sequences having the amino acid sequences of SEQ ID NOs:87, 183 and 279, respectively, and a V_{L} comprises the light chain CDR1, CDR2 and CDR3 having the amino acid sequences of SEQ ID NOs: 56, 152 and 248, respectively.
